(19) United States
(12) Patent Application Publication
(10) Pub. No.: US 2018/0201614 A1
(43) Pub. Date: Jul. 19, 2018

(54) BICYCLIC COMPOUNDS

(71) Applicant: KALYRA PHARMACEUTICALS, INC., San Diego, CA (US)

(72) Inventors: Kevin Duane Bunker, Escondido, CA (US); Sunny Abraham, San Diego, CA (US); Chad Daniel Hopkins, San Diego, CA (US); Joseph Robert Pinchman, San Diego, CA (US); Peter Qinhua Huang, San Diego, CA (US); Deborah Helen Slee, Encinitas, CA (US)

(21) Appl. No.: 15/572,744

(22) PCT Filed: May 10, 2016
(86) PCT No.: PCT/US2016/031663

§ 371 Date: Nov. 8, 2017

Related U.S. Application Data
(60) Provisional application No. 62/160,413, filed on May 12, 2015.

(51) Int. Cl. C07D 473/30; C07D 471/04; C07D 487/04; A61P 35/00

(52) U.S. Cl. CPC C07D 473/30; A61P 35/00; C07D 487/04; C07D 471/04

(57) ABSTRACT

Disclosed herein are nitrogen-containing bicyclic compounds, together with pharmaceutical compositions and methods of ameliorating and/or treating a cancer described herein with one or more of the compounds described herein.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6.

### BACKGROUND

### Field

The present application relates to the fields of chemistry, biochemistry and medicine. More particularly, disclosed herein are EGFR inhibitor compounds, together with pharmaceutical compositions, and methods of synthesizing the same. Also disclosed herein are methods of ameliorating and/or treating a cancer with one or more of the compounds described herein.

### Description

Overexpression of the EGFR gene has been identified in a variety of cancers including head and neck, brain, breast, colon and lung. In addition to overexpression, EGFR activating mutations have been detected in a subset of non-small cell lung cancers (NSCLCs) tumors. The majority of patients who respond well to first and second-generation EGFR inhibitors eventually develop resistance to these inhibitors. The most common resistance mechanism is an acquired gatekeeper mutation of threonine-to-methionine (T790M) in the EGFR gene. EGFR overexpression or activation, and acquired EGFR T790M mutation is observed in human cancers and is associated with high rates of cancer cell proliferation and drug resistance.

### SUMMARY

Some embodiments disclosed herein relate to a compound of Formula (I), or a pharmaceutically acceptable salt thereof.

Some embodiments described herein relate to a pharmaceutical composition, that can include an effective amount a compound of Formula (I), or a pharmaceutically acceptable salt thereof.

Some embodiments described herein relate to a method for ameliorating and/or treating a cancer described herein that can include administering an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for ameliorating and/or treating a cancer described herein. Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for ameliorating and/or treating a cancer described herein.

Some embodiments described herein relate to a method for inhibiting replication of a malignant growth or a tumor that can include contacting the growth or the tumor with an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof), wherein the malignant growth or tumor is due to a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for inhibiting replication of a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for inhibiting replication of a malignant growth or a tumor, wherein the malignant growth or tumor is due to a cancer described herein.

Some embodiments described herein relate to a method for ameliorating or treating a cancer described herein that can include contacting a malignant growth or a tumor with an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for ameliorating or treating a cancer described herein that can include contacting a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for ameliorating or treating a cancer described herein that can include contacting a malignant growth or a tumor, wherein the malignant growth or tumor is due to a cancer described herein.

Some embodiments described herein relate to a method for inhibiting the activity of EGFR (for example, inhibiting the activity of EGFR with acquired EGFR T790M mutation, or wildtype EGFR and where EGFR is overexpressed or activated) that can include providing an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) to a sample that includes a cancer cell from a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for inhibiting the activity of EGFR (for example, inhibiting the activity of EGFR with acquired EGFR T790M mutation, or wildtype EGFR and where EGFR is overexpressed or activated). Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for inhibiting the activity of EGFR (for example, inhibiting the activity of EGFR with acquired EGFR T790M mutation, or wildtype EGFR and where EGFR is overexpressed or activated).

Some embodiments described herein relate to a method for ameliorating or treating a cancer described herein that can include inhibiting the activity of EGFR (for example, inhibiting the activity of EGFR with acquired EGFR T790M mutation, or wildtype EGFR and where EGFR is overexpressed or activated) using an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for ameliorating or treating a cancer described herein by inhibiting the activity of EGFR (for example, inhibiting the activity of EGFR with acquired EGFR T790M mutation, or wildtype EGFR and where EGFR is overexpressed or activated). Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for ameliorating or treating a cancer described herein by inhibiting the activity of EGFR (for example, inhibiting the activity of EGFR with acquired EGFR T790M mutation, or wildtype EGFR and where EGFR is overexpressed or activated).

### DETAILED DESCRIPTION

Inhibition of EGFR can have therapeutic effects in the treatment of cancer. It has been shown that EGFR can mutate and become activated, driving tumor growth. Epidermal growth factor receptor (EGFR) has an extracellular ligand binding domain, a transmembrane portion, and intracellular tyrosine kinase and regulatory domains. Upon binding of a specific ligand, EGFR undergoes conformational change and phosphorylation of the intracellular domain occurs leading to downstream signal transduction that regulates cellular proliferation. Constitutive activation of EGFR leads to increased intracellular pathways activity which eventually leads to cell proliferation, angiogenesis, invasion and/or metastasis.

Overexpression of the EGFR gene has been identified in a variety of cancers including head and neck, brain, breast, colon and lung. In non-small cell lung cancer, the frequency of EGFR overexpression has been determined to be 40% to 80%. In addition to overexpression, EGFR activating mutations have been detected in a subset of non-small cell lung cancers (NSCLCs) tumors, which represent 10% to 30% of all NSCLCs. The mutations occur in exons 18, 19 and 21 of the tyrosine kinase domain of the EGFR gene. The majority of mutations in exon 21 are point mutations whereas exon 19 consists of almost entirely in-frame deletions. The L858R point mutation and the deletion in exon 19, account up to 86% of all EGFR mutations. These mutations result in increased kinase activity of the EGF receptor in the absence of growth factors. The above-mentioned mutations in EGF receptor were shown to be a predictive biomarker of efficacy in response to EGFR tyrosine kinase inhibitors. These findings have revolutionized the way in which EGFR inhibitors are used as therapy for NSCLC patients with activating EGFR mutations. The EGFR inhibitors, erlotinib and gefitinib (considered first generation EGFR inhibitors) were approved in the United States, initially as second-line therapies. However, subsequent clinical trials of EGFR inhibitors, including the first-generation EGFR inhibitors (gefitinib) and second-generation EGFR inhibitor, afatinib, demonstrated significant improvements in overall response rates in NSCLC patients with EGFR activating mutations in the frontline setting.

The majority of patients who respond well to the first and second-generation EGFR inhibitors eventually develop resistance to these inhibitors. The most common resistance mechanism, which is observed in approximately 50% of the patients, is an acquired gatekeeper mutation of threonine-to-methionine (T790M) in the EGFR gene. This mutation increases the receptor's affinity for ATP and decreases the effectiveness of first generation EGFR inhibitors. Therefore, the NSCLC patients who refract on first and second-generation EGFR inhibitors need new therapies that can overcome the acquired resistance associated with the T790M mutation.

Provided herein are compounds that can inhibit the kinase activity of EGFR. As EGFR inhibitors, the compounds described herein can be used to ameliorate and/or treat a variety of cancers (including those with acquired EGFR T790M mutation, or wildtype EGFR and where EGFR is overexpressed or activated) such as non-small cell lung, head and neck, brain, breast and colon cancer.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless the context indicates otherwise.

As used herein, any “R” group(s) such as, without limitation, R¹, R², R³, R⁴, R^{1A}, R^{1B}, R^{2A} and R^{2B} represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

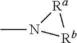

In addition, if two “R” groups are described as being “taken together” with the atom(s) to which they are attached to form a ring as an alternative, the R groups are not limited to the variables or substituents defined previously.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, acylalkyl, hydroxy, alkoxy, alkoxyalkyl, aminoalkyl, amino acid, aryl, heteroaryl, heterocyclyl, aryl(alkyl), heteroaryl(alkyl), heterocyclyl(alkyl), hydroxyalkyl, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, azido, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heteroalicyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused or bridged fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl, cyclooctyl, bicyclo[1.1.1]pentane, bicyclo[2.1.1]heptane, admantanyl and norbornyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s) or 3 to 8 atoms in the ring(s). When composed of two or more rings, the rings may be connected together in a fused or bridged fashion. A cycloalkenyl group may be unsubstituted or substituted.

As used herein, the term “fused” refers to a connectivity between two rings in which two adjacent atoms and one bond (saturated or unsaturated) are shared between the rings. For example, in the following structure, rings A and B are fused

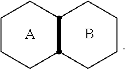

Examples of fused ring structures include, but are not limited to, decahydronaphthalene, 1H-indole, quinolone, chromane, bicyclo[2.1.0]pentane and 6,7,8,9-tetrahydro-5H-benzo[7]annulene.

As used herein, the term “bridged” refers to a connectivity wherein three or more atoms are shared between two rings. The following structures

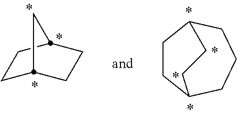 [SMILES: C1C[C@H]2CC[C@H]1C2 ; C1CC2CCC(C1)CC2]

are examples of “bridged” rings because the indicated atoms are shared between at least two rings. Examples of bridged ring structures include, but are not limited to, bicyclo[1.1.1]pentane, 2-oxabicyclo[1.1.1]pentane, 5-azabicyclo[2.1.1]hexane, 6-azabicyclo[3.1.1]heptane, adamantane and norbornane.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one, two, three or more heteroatoms (for example, 1, 2, 3, 4 or 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems. Examples of heteroaryl rings include, but are not limited to, those described herein and the following: furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl or heteroalicyclic groups may be unsubstituted or substituted. Examples of such “heterocyclyl” or “heteroalicyclyl” groups include, but are not limited to, those described herein and the following: 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 1,3-thiazinane, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidone, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline and 3,4-methylenedioxyphenyl). Examples of bridged heterocyclic compounds include, but are not limited to, 1,4-diazabicyclo[2.2.2]octane and 1,4-diazabicyclo[3.1.1]heptane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, imidazolylalkyl and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a heteroalicyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl), and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “acylalkyl” refers to an acyl connected, as a substituent, via a lower alkylene group. Examples include aryl-C(═O)—(CH₂)ₙ— and heteroaryl-C(═O)—(CH₂)ₙ—, where n is an integer in the range of 1 to 6.

As used herein, “alkoxyalkyl” refers to an alkoxy group connected, as a substituent, via a lower alkylene group. Examples include C₁₋₄ alkyl-O—(CH₂)ₙ—, wherein n is an integer in the range of 1 to 6.

As used herein, “aminoalkyl” refers to an optionally substituted amino group connected, as a substituent, via a lower alkylene group. Examples include H₂N(CH₂)ₙ—, wherein n is an integer in the range of 1 to 6.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl and 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkyl, chloro-difluoroalkoxy and 2-fluoroisobutoxy. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “carbonyl” group refers to a C═O group.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

The term “halogen atom” or “halogen” as used herein, means any one of the radio-stable atoms of column 7 of the Periodic Table of the Elements, such as, fluorine, chlorine, bromine and iodine.

An “activated alkenyl” is used herein as understood by those skilled in the art, and refers to an alkenyl that is substituted with at least one electron withdrawing group. Examples of suitable electron withdrawings groups are an optionally substituted acyl, an optionally substituted C-carboxy, an optionally substituted C-amido, an optionally substituted N-amido, an optionally substituted phosphate, an optionally substituted sulfinyl, and an optionally substituted sulfonyl, cyano and nitro. An example of an activated alkenyl is a Michael acceptor.

As used herein, “ [symbol] ” indicates a single or double bond, unless stated otherwise.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

The term “leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of strong acids. Examples of suitable leaving groups include, but are not limited to, tosylates, mesylates, trifluoroacetates and halogens (e.g., I, Br, and Cl). Non-limiting characteristics and examples of leaving groups can be found, for example in Organic Chemistry, 2d ed., Francis Carey (1992), pages 328-331; Introduction to Organic Chemistry, 2d ed., Andrew Streitwieser and Clayton Heathcock (1981), pages 169-171; and Organic Chemistry, 5^{th} ed., John McMurry (2000), pages 398 and 408; all of which are incorporated herein by reference for the limited purpose of disclosing characteristics and examples of leaving groups.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless the context indicates otherwise. Similarly, a group of items linked with the conjunction ‘or’ should not be read as requiring mutual exclusivity among that group, but rather should be read as ‘and/or’ unless the context indicates otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Compounds

### Formula (I)

Some embodiments disclosed herein relate to a compound of Formula (I), or a pharmaceutically acceptable salt thereof, having the structure:

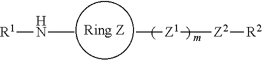

wherein: Ring Z can be selected from:

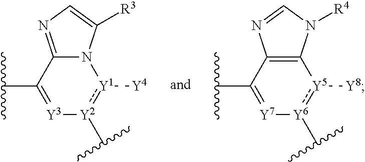

each [symbol] can be independently a single or double bond; Y¹ can be C (carbon) or N (nitrogen); wherein Y² is N (nitrogen), the [symbol] between Y¹ and Y² can be a single bond, Y¹ can be C (carbon), the ----- bond to Y⁴ can be a double bond and Y⁴ can be O (oxygen); or wherein Y² is C (carbon), the [symbol] between Y¹ and Y² can be a double bond, Y¹ can be N (nitrogen), the ----- bond can be absent and Y⁴ can be absent; or wherein Y² is C (carbon), the [symbol] between Y¹ and Y² can be a double bond, Y¹ can be C (carbon), the ----- bond to Y⁴ can be a single bond and Y⁴ can be selected from hydrogen, halogen, an optionally substituted C₁₋₄ alkyl, an optionally substituted cycloalkyl, an optionally substituted alkoxy, an optionally substituted mono-substituted amine and an optionally substituted disubstituted amine; Y³ is CR^{1A} or N (nitrogen); Y⁵ is C (carbon) or N (nitrogen); wherein Y⁶ is N (nitrogen), the [symbol] between Y⁵ and Y⁶ can be a single bond, Y⁵ can be C (carbon), the ----- bond to Y⁸ can be a double bond and Y⁸ can be O (oxygen); or wherein Y⁶ is C, the [symbol] between Y⁵ and Y⁶ can be a double bond, Y⁵ can be N (nitrogen), the ----- bond can be absent and Y can be absent; or wherein Y⁶ is C (carbon), the [symbol] between Y⁵ and Y⁶ can be a double bond, Y⁵ can be C (carbon), the ----- bond to Y⁸ can be a single bond and Y⁸ can be selected from hydrogen, halogen, an optionally substituted C₁₋₄ alkyl, an optionally substituted cycloalkyl, an optionally substituted alkoxy, an optionally substituted mono-substituted amine and an optionally substituted disubstituted amine; Y⁷ can be CR^{1B} or N (nitrogen); R¹ can be an optionally substituted aryl or an optionally substituted heteroaryl; R² can be selected from a substituted C₄-C₁₀ cycloalkyl, a substituted aryl, a substituted heteroaryl and a substituted heterocyclyl, and wherein R² can be substituted with an activated alkenyl; R³ and R⁴ can be independently selected from hydrogen, halogen, an optionally substituted C₁₋₄ alkyl, an optionally substituted C₃-C₁₀ cycloalkyl, an optionally substituted alkoxy, an optionally substituted mono-substituted sulfenyl, an optionally substituted mono-substituted amine and an optionally substituted disubstituted amine; R^{1A} and R^{1B} can be independently selected from hydrogen, halogen, an optionally substituted C₁₋₄ alkyl, an optionally substituted cycloalkyl, an optionally substituted alkoxy, an optionally substituted mono-substituted amine and an optionally substituted disubstituted amine; Z¹ can be O (oxygen), S (sulfur) or NH; Z² can be (CR^{2A}R^{2B})ₙ; R^{2A} and R^{2B} can be independently selected from hydrogen, halogen, an optionally substituted C₁₋₄ alkyl, an optionally substituted C₁₋₄ alkoxy and an optionally substituted C₁₋₄ haloalkyl; m can be 0 or 1; and n can be 0, 1, 2 or 3.

Ring Z can be a variety of bicyclic ring systems that contain several nitrogen atoms. In some embodiments, ring Z can be

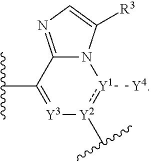

In other embodiments, ring Z can be

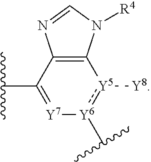

When ring Z is

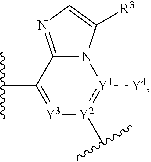

in some embodiments, Y² can be N (nitrogen), the [symbol] between Y¹ and Y² can be a single bond, Y¹ can be C (carbon), the ----- bond to Y⁴ can be a double bond and Y⁴ can be O (oxygen). In other embodiments, Y² can be C (carbon), the [symbol] between Y¹ and Y² can be a double bond, Y¹ can be N (nitrogen), the ----- bond can be absent and Y⁴ can be absent. In still other embodiments, Y² can be C (carbon), the [symbol] between Y¹ and Y² can be a double bond, Y¹ can be C (carbon), the ----- bond to Y⁴ can be a single bond and Y⁴ can be selected from hydrogen, halogen, an optionally substituted C₁₋₄ alkyl, an optionally substituted cycloalkyl, an optionally substituted alkoxy, an optionally substituted mono-substituted amine and an optionally substituted disubstituted amine. In some embodiments, Y³ can be CR^{1A}. In other embodiments, Y³ can be N (nitrogen).

Examples of ring Z having the structure

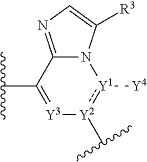

include the following:

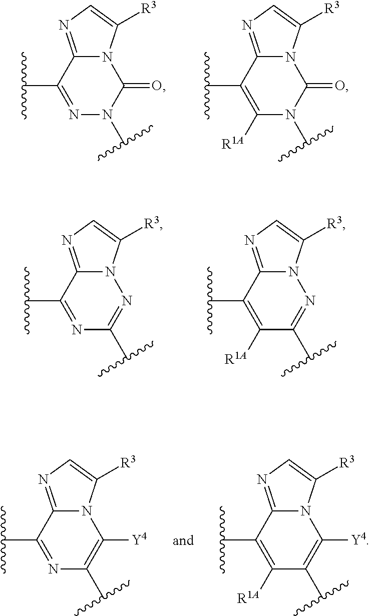

In some embodiments, including those of the previous paragraphs, R^{1A} can be hydrogen. In other embodiments, including those of the previous paragraphs, R^{1A} can be halogen. In still other embodiments, including those of the previous paragraphs, R^{1A} can be an optionally substituted C₁₋₄ alkyl, such as those described herein. In some embodiments, R^{1A} can be an unsubstituted C₁₋₄ alkyl. In other embodiments, R^{1A} can be a substituted C₁₋₄ alkyl. In some embodiments, including those of the previous paragraphs, R^{1A} can be an optionally substituted cycloalkyl, such as an optionally substituted monocyclic C₃₋₈ cycloalkyl or an optionally substituted bicyclic C₃₋₈ cycloalkyl. In other embodiments, including those of the previous paragraphs, R^{1A} can be an optionally substituted alkoxy, for example, an optionally substituted C₁₋₄ alkoxy. In still other embodiments, including those of the previous paragraphs, R^{1A} can be an optionally substituted mono-substituted amine. In yet still other embodiments, including those of the previous paragraphs, R^{1A} can be an optionally substituted disubstituted amine.

A variety of substituents can be attached to ring Z. In some embodiments, R³ can be hydrogen. In other embodiments, R³ can be halogen. In still other embodiments, R³ can be an optionally substituted C₁₋₄ alkyl. Examples of C₁₋₄ alkyl groups include methyl, ethyl, n-propyl, iso-propyl, n-butyl, iso-butyl and tert-butyl. In some embodiments, R³ can be an unsubstituted C₁₋₄ alkyl. In other embodiments, R³ can be a substituted C₁₋₄ alkyl.

In some embodiments, R³ can be an unsubstituted C₃-C₁₀ cycloalkyl. In other embodiments, R³ can be a substituted C₃-C₁₀ cycloalkyl. The C₃-C₁₀ cycloalkyl can be a mono-cyclic C₃-C₁₀ cycloalkyl or a bicyclic C₃-C₁₀ cycloalkyl, such as a fused C₃-C₁₀ cycloalkyl. Examples of C₃-C₁₀ cycloalkyl groups include, but not limited to, cyclopropyl, cyclobutyl, cyclopentyl, cyclohexyl, cycloheptyl, cyclooctyl, bicyclo[1.1.1]pentyl and bicyclo[2.1.1]heptyl. In some embodiments, the C₃-C₁₀ cycloalkyl can be bicyclo[1.1.1]pentyl moiety.

In some embodiments, R³ can be an unsubstituted alkoxy. In other embodiments, R³ can be a substituted alkoxy. In some embodiments, R³ can be an optionally substituted C₁₋₄ alkoxy. As one example, R³ can be an unsubstituted methoxy. In some embodiments, R³ can be an optionally substituted mono-substituted sulfenyl. In other embodiments, R³ can be an optionally substituted mono-substituted amine. For example, R³ can be —NHR″, wherein R″ can be an optionally substituted C₁₋₄ alkyl. In still other embodiments, R³ can be an optionally substituted disubstituted amine.

As provided herein, ring Z can have the structure

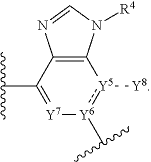

In some embodiments, Y⁶ can be N (nitrogen), the [symbol] between Y⁵ and Y⁶ can be a single bond, Y⁵ can be C (carbon), the ----- bond to Y⁸ can be a double bond and Y⁸ can be O (oxygen). In other embodiments, Y⁶ can be C (carbon), the [symbol] between Y⁵ and Y⁶ can be a double bond, Y⁵ can be N (nitrogen), the ----- bond can be absent and Y⁸ can be absent. In still other embodiments, Y⁶ can be C (carbon), the [symbol] between Y⁵ and Y⁶ can be a double bond, Y⁵ can be C (carbon), the ----- bond to Y⁸ can be a single bond and Y⁸ can be selected from hydrogen, halogen, an optionally substituted C₁₋₄ alkyl, an optionally substituted cycloalkyl, an optionally substituted alkoxy, an optionally substituted mono-substituted amine and an optionally substituted disubstituted amine. In some embodiments, Y⁷ can be CR^{1B}. In other embodiments, Y⁷ can be N (nitrogen). Examples of ring Z having the structure

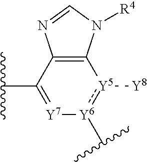

include the following:

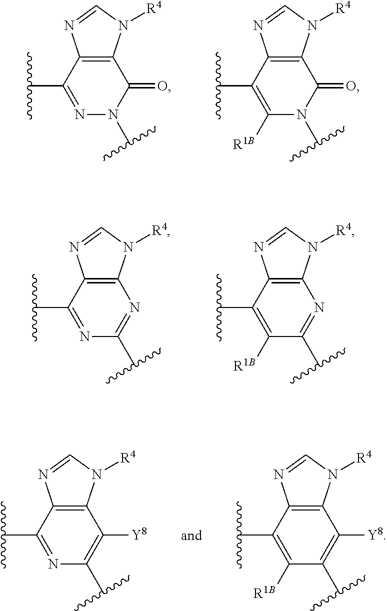

In some embodiments, including those of the previous paragraphs, R^{1B} can be hydrogen. In other embodiments, including those of the previous paragraphs, R^{1B} can be halogen. In still other embodiments, including those of the previous paragraphs, R^{1B} can be an optionally substituted C₁₋₄ alkyl, such as those described herein. In some embodiments, R^{1B} can be an unsubstituted C₁₋₄ alkyl. In other embodiments, R^{1B} can be a substituted C₁₋₄ alkyl. In some embodiments, including those of the previous paragraphs, R^{1B} can be an optionally substituted cycloalkyl, such as an optionally substituted monocyclic C₃₋₈ cycloalkyl or an optionally substituted bicyclic C₃₋₈ cycloalkyl. In other embodiments, including those of the previous paragraphs, R^{1B} can be an optionally substituted alkoxy, for example, an optionally substituted C₁₋₄ alkoxy. In still other embodiments, including those of the previous paragraphs, R^{1B} can be an optionally substituted mono-substituted amine. In yet still other embodiments, including those of the previous paragraphs, R^{1B} can be an optionally substituted disubstituted amine.

In some embodiments, R⁴ can be hydrogen. In other embodiments, R⁴ can be halogen. In still other embodiments, R⁴ can be an optionally substituted C₁₋₄ alkyl. Examples of C₁₋₄ alkyl groups include methyl, ethyl, n-propyl, iso-propyl, n-butyl, iso-butyl and tert-butyl. In some embodiments, R⁴ can be an unsubstituted C₁₋₄ alkyl. In other embodiments, R⁴ can be a substituted C₁₋₄ alkyl.

In some embodiments, R⁴ can be an unsubstituted C₃-C₁₀ cycloalkyl. In other embodiments, R⁴ can be a substituted C₃-C₁₀ cycloalkyl. The C₃-C₁₀ cycloalkyl can be a mono-cyclic C₃-C₁₀ cycloalkyl or a bicyclic C₃-C₁₀ cycloalkyl, such as a fused C₃-C₁₀ cycloalkyl. Examples of C₃-C₁₀ cycloalkyl groups include, but not limited to, cyclopropyl, cyclobutyl, cyclopentyl, cyclohexyl, cycloheptyl, cyclooctyl, bicyclo[1.1.1]pentyl and bicyclo[2.1.1]heptyl. In some embodiments, the C₃-C₁₀ cycloalkyl can be bicyclo[1.1.1]pentyl moiety.

In some embodiments, R⁴ can be an unsubstituted alkoxy. In other embodiments, R⁴ can be a substituted alkoxy. In some embodiments, R⁴ can be an optionally substituted C₁₋₄ alkoxy. For example, R⁴ can be an unsubstituted methoxy. In some embodiments, R³ can be an optionally substituted mono-substituted sulfenyl. In other embodiments, R⁴ can be an optionally substituted mono-substituted amine. As one example, R⁴ can be —NHR″, wherein R″ can be an optionally substituted C₁₋₄ alkyl. In still other embodiments, R⁴ can be an optionally substituted disubstituted amine.

In some embodiments, R¹ can be an optionally substituted aryl. For example, R¹ can be an optionally substituted phenyl. The aryl ring can be substituted 1 or more times with a variety of substituents. For example, the aryl ring can be substituted with 2, 3 or more than 3 substituents. The substituents can be the same or different from each other. In other embodiments, R¹ can be an optionally substituted heteroaryl. The optionally substituted heteroaryl can be an optionally substituted monocyclic heteroaryl or an optionally substituted bicyclic heteroaryl. Various optionally substituted heteroaryl groups include, but are not limited to, an optionally substituted pyrazole, an optionally substituted pyridine, an optionally substituted pyrimidine, an optionally substituted imidazole, an optionally substituted thiazole, an optionally substituted isoxazole, an optionally substituted oxazole and an optionally substituted triazole. The heteroaryl group can be a mono-substituted, di-substituted or substituted with 3 or more substituents. When R¹ is substituted, one or more of the following substituents can be present: halogen, an optionally substituted C₁₋₄ alkyl, an optionally substituted C₃₋₈ cycloalkyl, an optionally substituted mono-cyclic heterocyclyl, an optionally substituted C₁₋₄ alkoxy, an optionally substituted C₁₋₄ haloalkyl, an optionally substituted mono-substituted amine and an optionally substituted disubstituted amine, wherein when any of the aforementioned are substituted, one or more of the following substituents can be present: halogen, an unsubstituted C₁₋₄ alkyl, an unsubstituted C₁₋₄ haloalkyl and an amine substituted with one to two unsubstituted C₁₋₄ alkyl groups. When R¹ is substituted with an optionally substituted mono-cyclic heterocyclyl, the optionally substituted mono-cyclic heterocyclyl can be an optionally substituted nitrogen-containing mono-cyclic heterocyclyl, such as and an optionally substituted pyrrolidinyl and an optionally substituted piperidinyl.

In some embodiments, R² can be a substituted C₄-C₁₀ cycloalkyl. The substituted C₄-C₁₀ cycloalkyl can be, for example, a substituted monocyclic C₄₋₆ cycloalkyl or a substituted bicyclic C₅₋₁₀ cycloalkyl. The bicyclic C₅₋₁₀ cycloalkyl can be a fused C₅₋₁₀ cycloalkyl, such as bicyclo[1.1.1]pentyl or bicyclo[2.1.1]heptyl.

In some embodiments, R² can be a substituted aryl. For example, R² can be substituted phenyl. In some embodiments, R² can be a mono-substituted phenyl group substituted at the ortho, meta or para position. In other embodiments, R² can be a substituted aryl group with 2, 3 or more than 3 substituents. When 2 or more substituents are present, the substituents can be the same or different from each other. In some embodiments, R² can be a substituted heteroaryl. Examples of substituted heteroaryls include a substituted monocyclic heteroaryl and a substituted bicyclic heteroaryl. In still other embodiments, R² can be a substituted heterocyclyl. The substituted heterocyclyl can be a substituted monocyclic heterocyclyl or a substituted bicyclic heterocyclyl. In some embodiments, R² can be selected from a substituted pyrrolidinyl, a substituted piperidine and 3-azabicyclo[3.1.0]hexanyl.

The activated alkenyl attached to R² can have a variety of structures. In some embodiments, the activated alkenyl can be an C₂₋₆ alkenyl that can include a moiety selected from an optionally substituted acyl, an optionally substituted C-carboxy, an optionally substituted C-amido, cyano and nitro. In some embodiments, the activated alkenyl can be an optionally substituted —C(═O)—C₂₋₄ alkenyl. In other embodiments, the activated alkenyl can be an optionally substituted —NR⁵—C(═O)—C₂₋₄ alkenyl, wherein R⁵ can be hydrogen or an optionally substituted C₁₋₄ alkyl. Examples of suitable activated alkenyls include, but are not limited to,

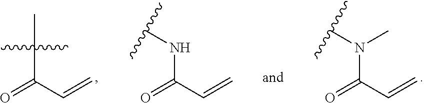

In some embodiments, R² can be an optionally substituted moiety selected from:

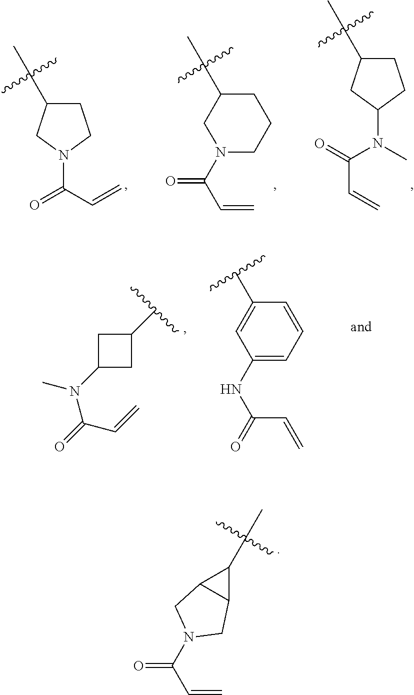

In addition to the activated alkenyl present on R², one or more of the following substituents can be present on R²: halogen, an optionally substituted C₁₋₄ alkyl, an optionally substituted C₃₋₈ cycloalkyl, an optionally substituted C₁₋₄ alkoxy, an optionally substituted C₁₋₄ haloalkyl, an optionally substituted mono-substituted amine and an optionally substituted disubstituted amine.

In some embodiments, Z¹ can be O (oxygen). In other embodiments, Z¹ can be S (sulfur). In still other embodiments, Z¹ can be NH. In some embodiments, m can be 0. In other embodiments, m can be 1.

In some embodiments, Z² can be (CR^{2A}R^{2B})n, and R^{2A} and R^{2B} can be independently selected from hydrogen, halogen, an optionally substituted C₁₋₄ alkyl, an optionally substituted C₁₋₄ alkoxy and an optionally substituted C₁₋₄ haloalkyl. In some embodiments, one of R^{2A} and R^{2B} can be hydrogen. In other embodiments, both of R^{2A} and R^{2B} can be hydrogen, such that Z² can be (CH₂)n. In some embodiments, n can be 0. In other embodiments, n can be 1. In still other embodiments, n can be 2. In yet still other embodiments, n can be 3.

Examples of compounds of Formula (I), or a pharmaceutically acceptable salt thereof, include, but are not limited to:

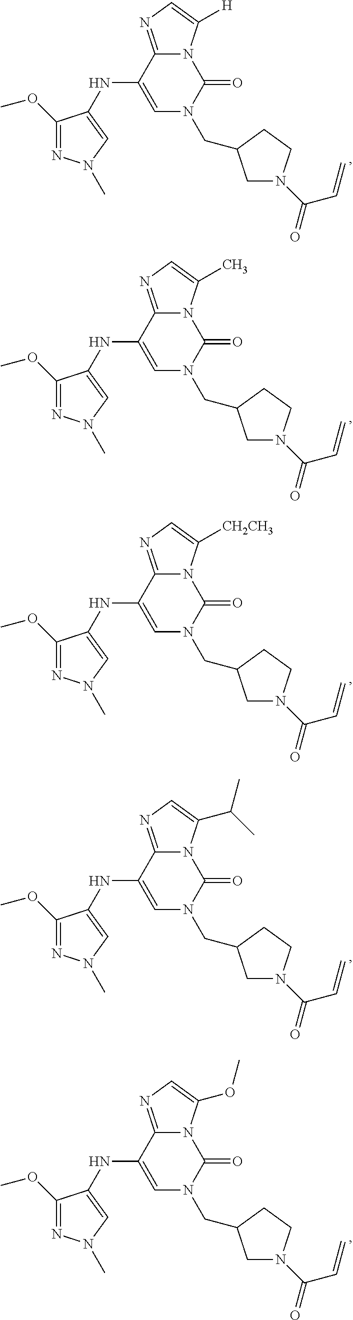

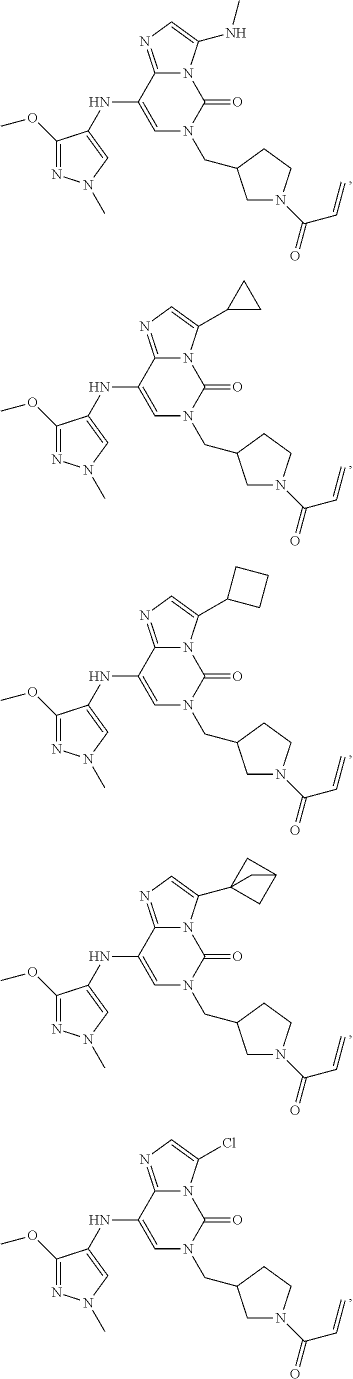

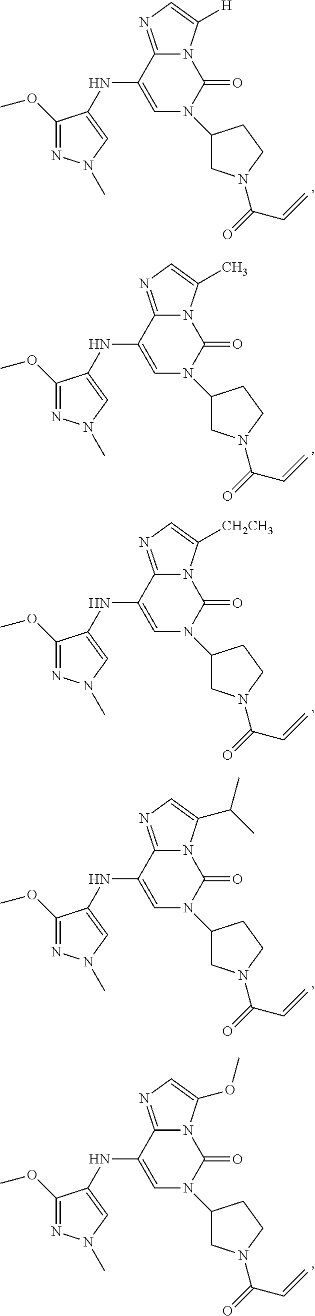

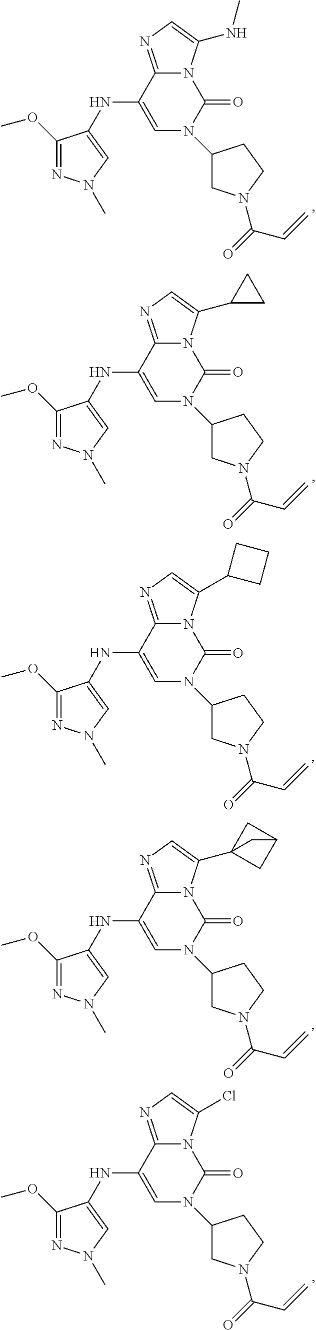

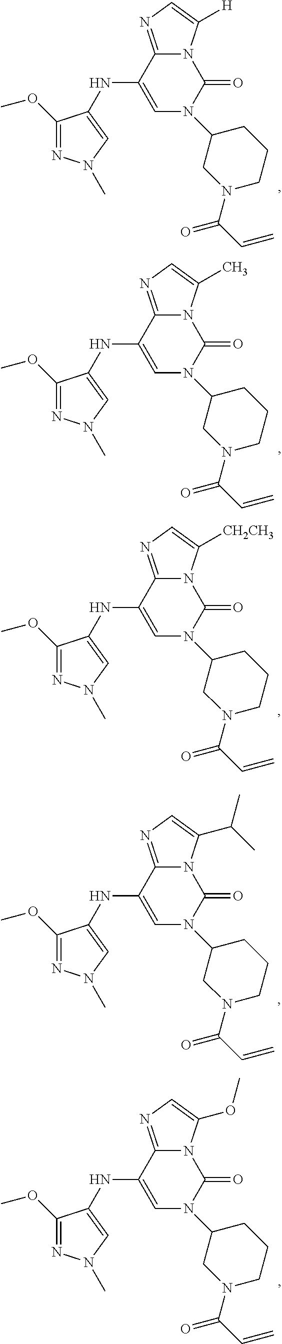

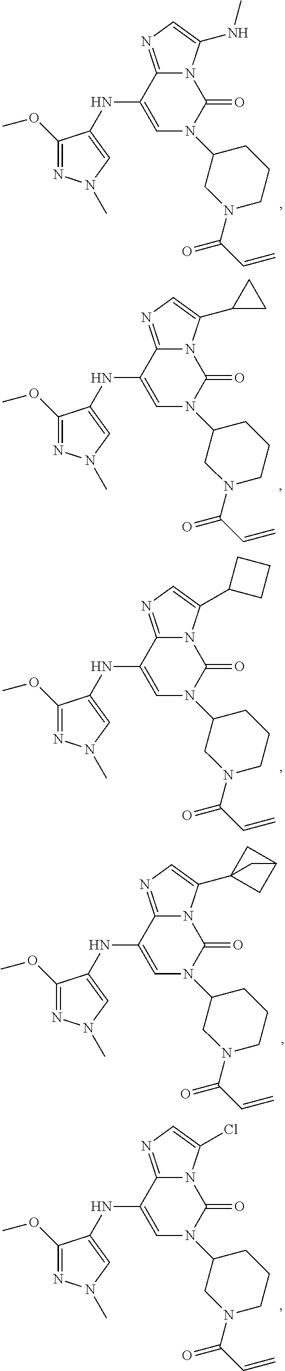

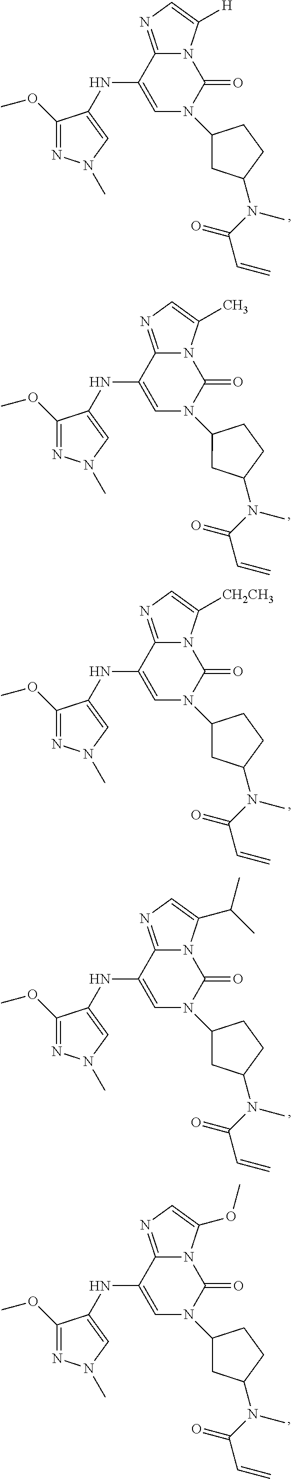

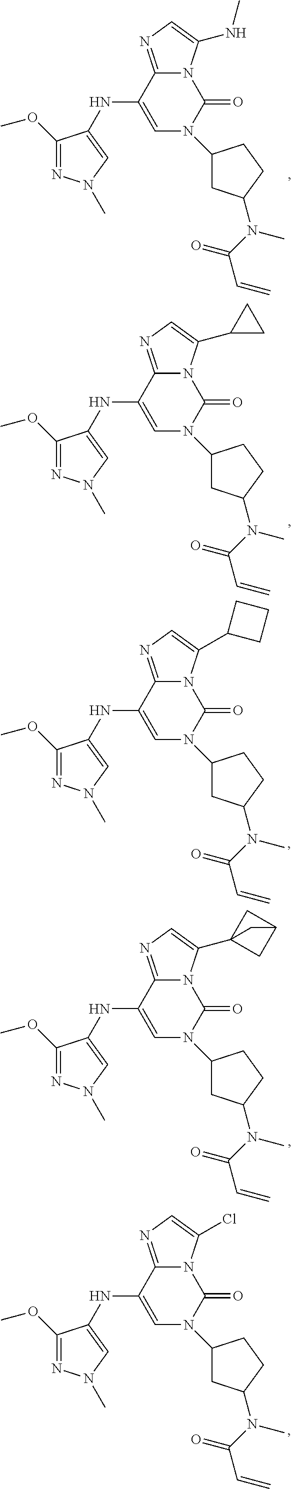

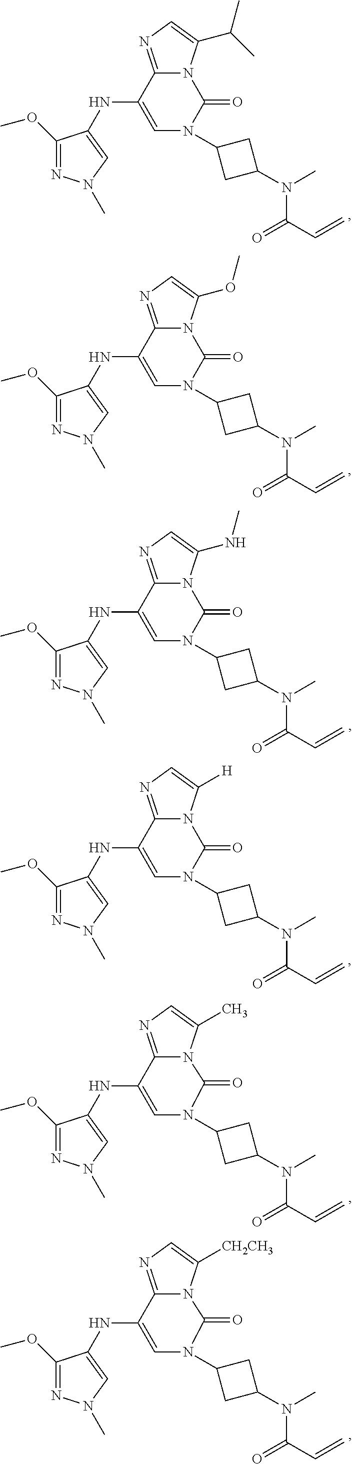

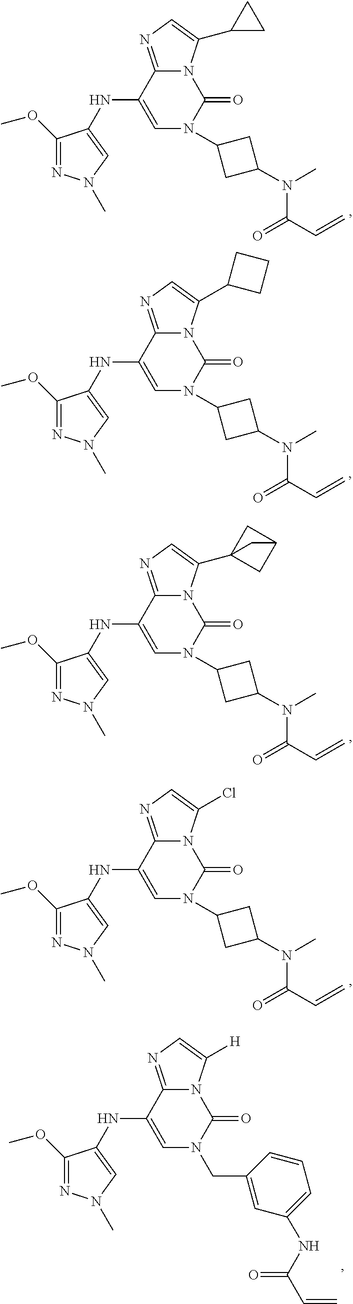

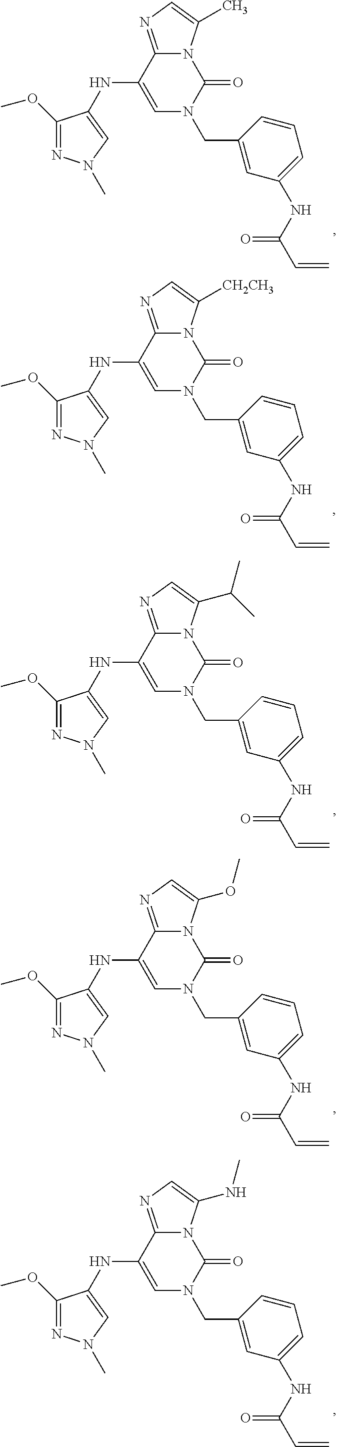

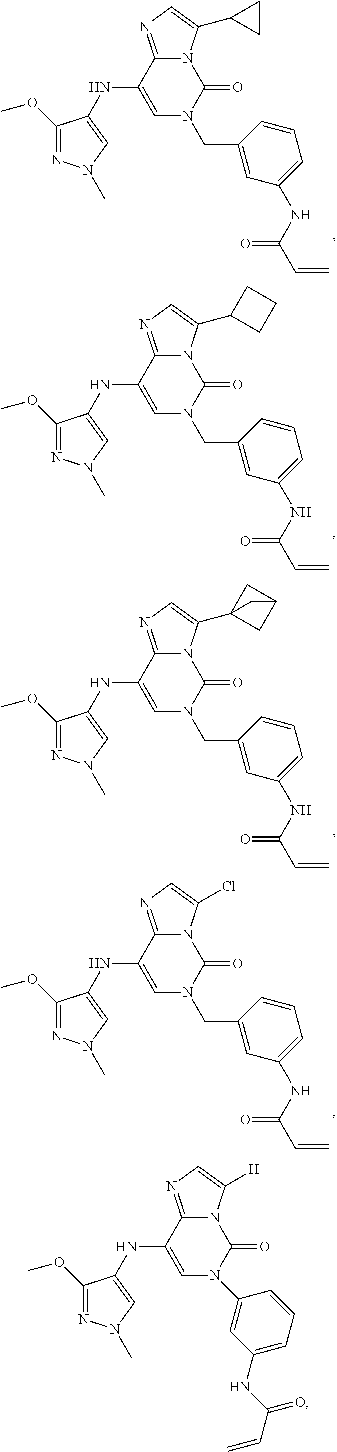

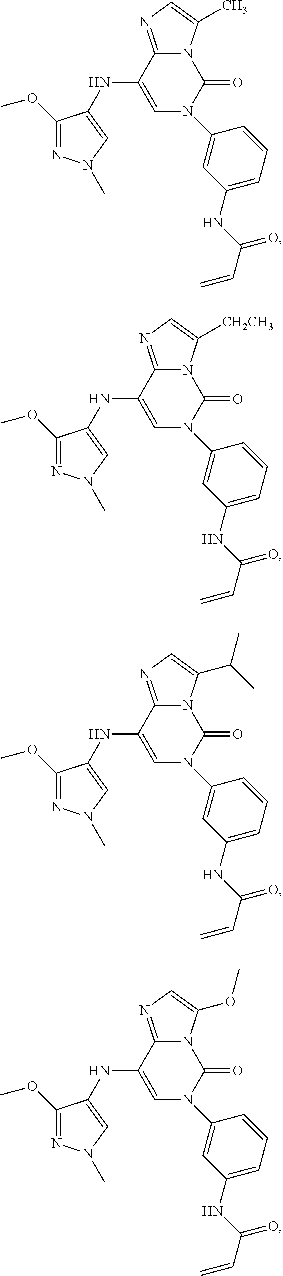

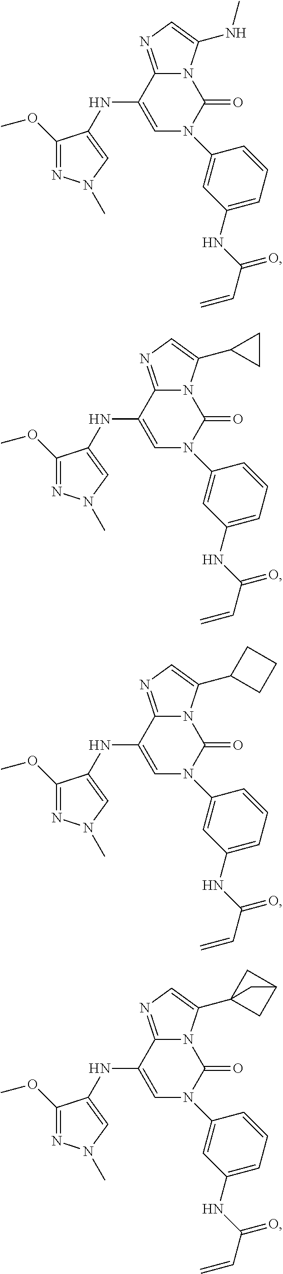

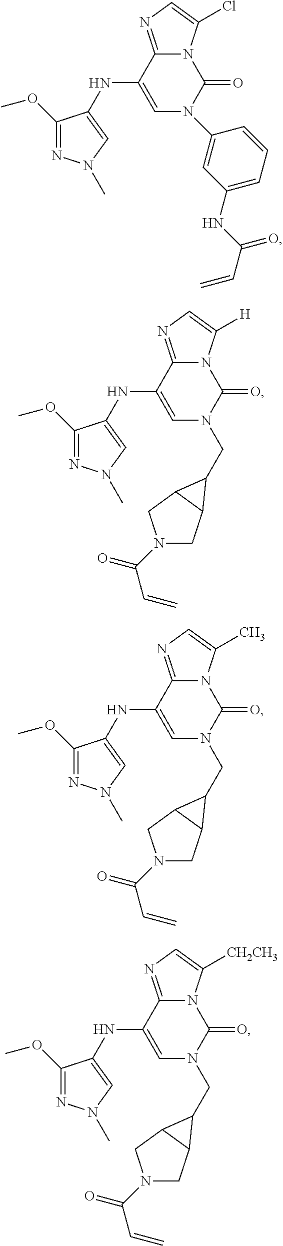

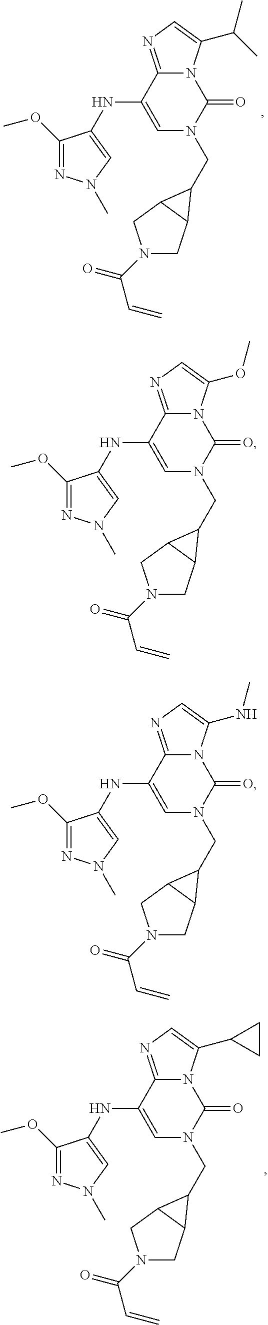

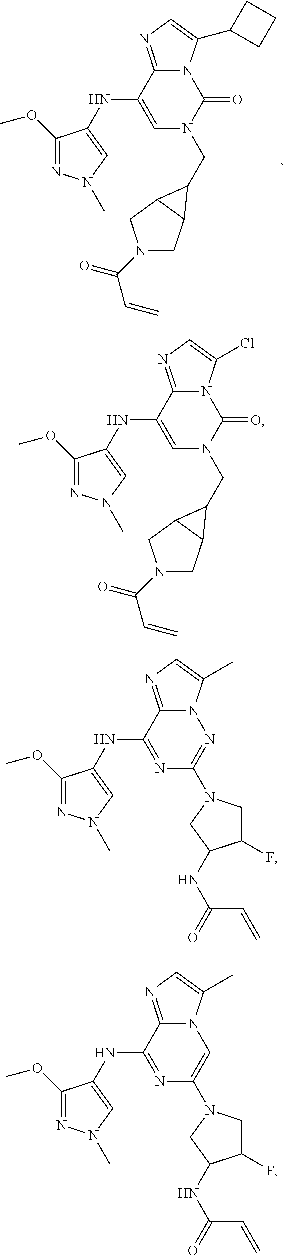

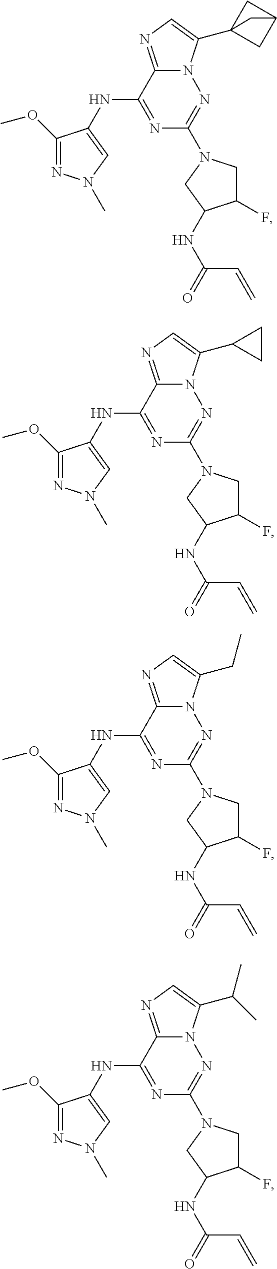

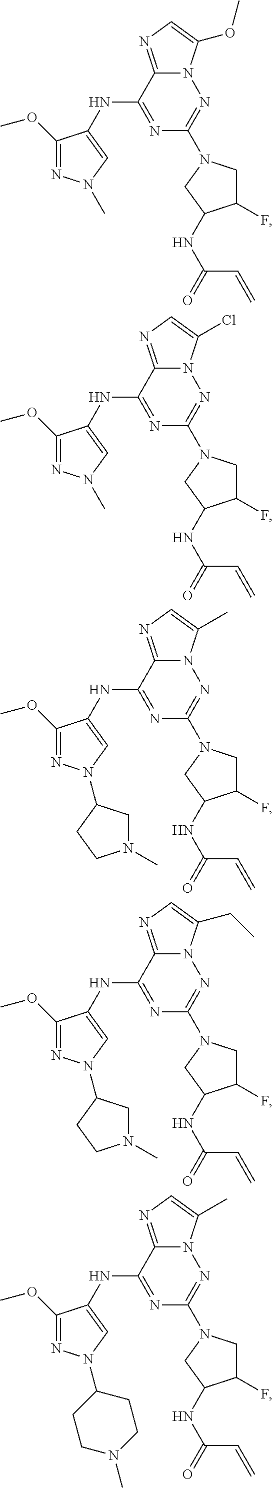

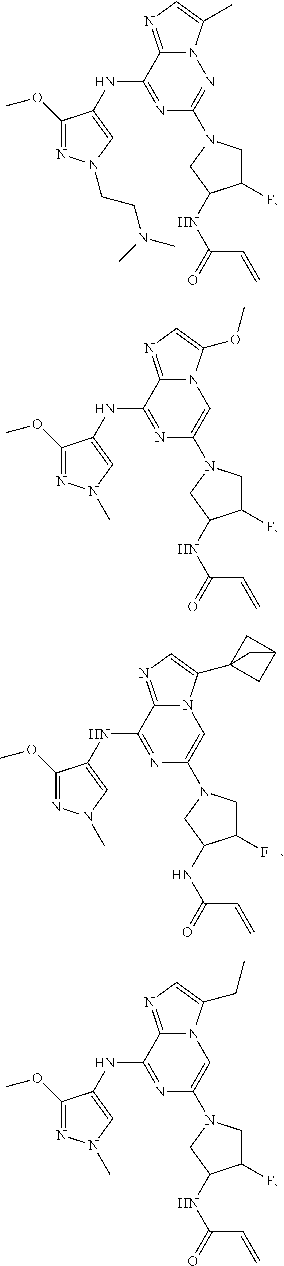

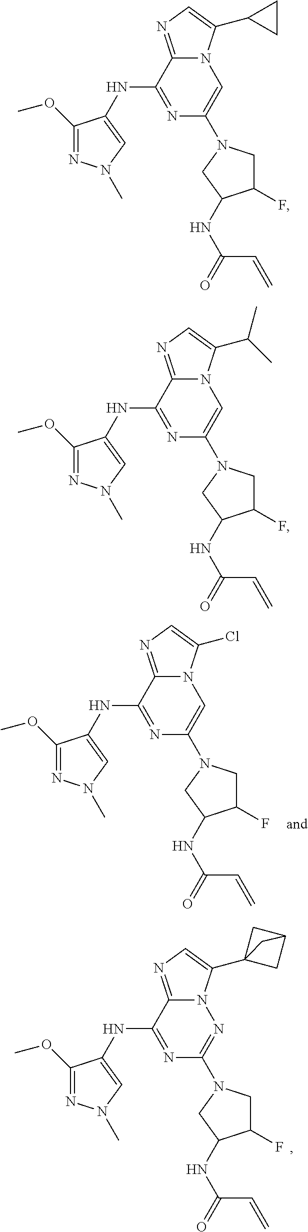

or a pharmaceutically acceptable salt of any of the foregoing.

Additional examples of compounds of Formula (I), or a pharmaceutically acceptable salt thereof, include the following:

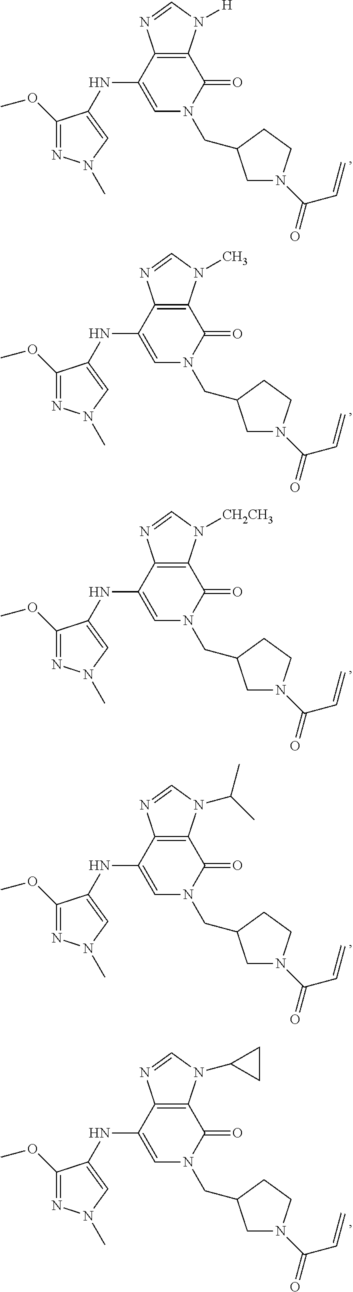

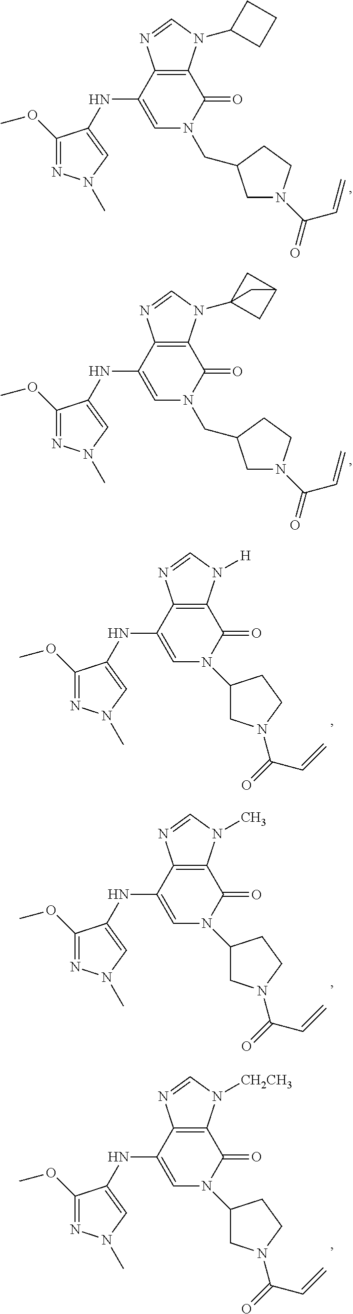

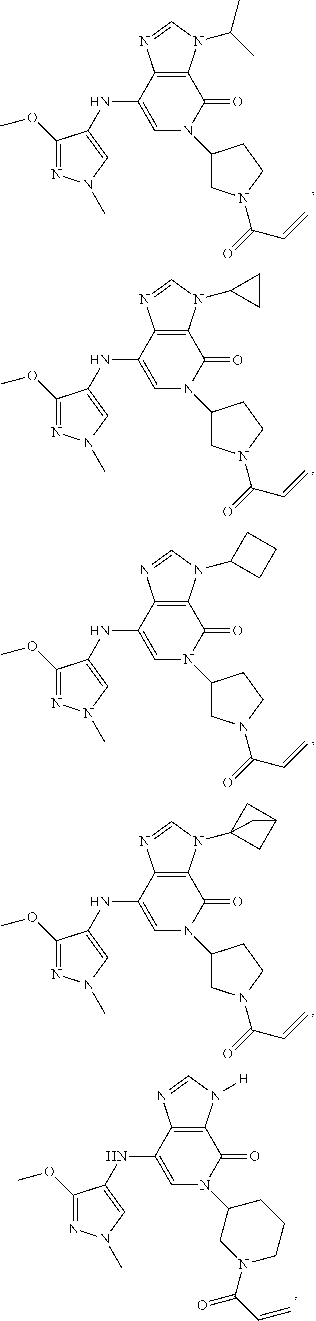

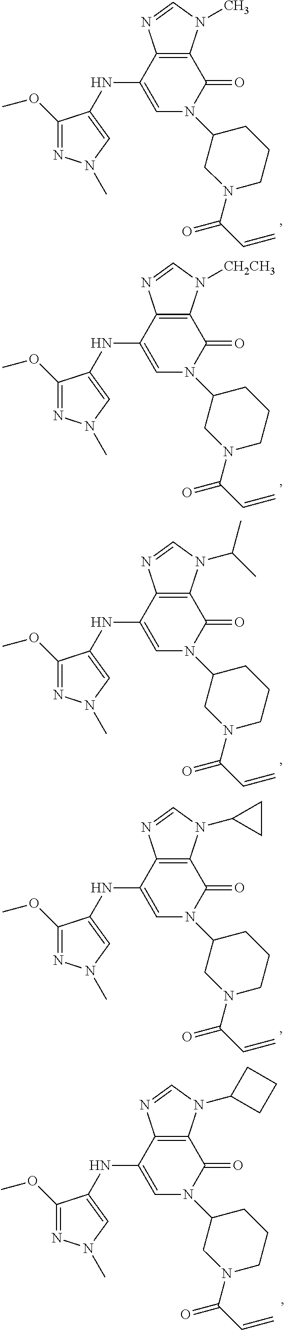

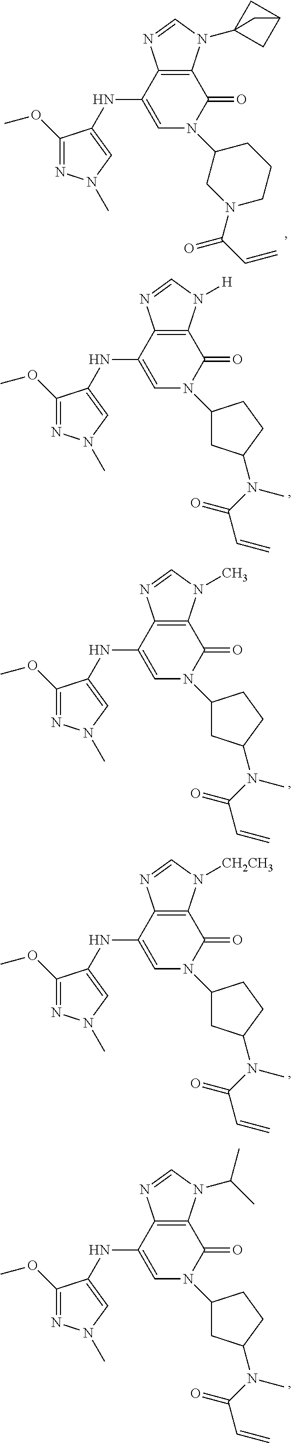

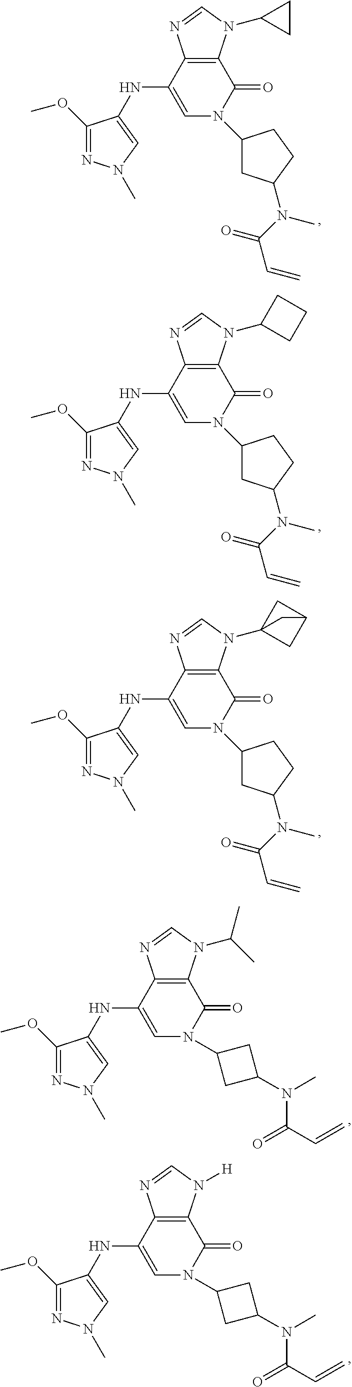

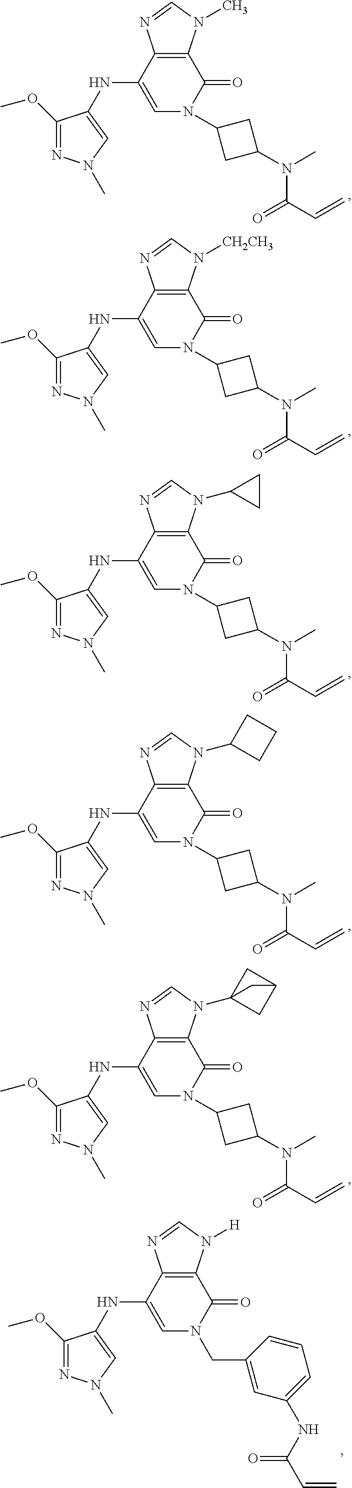

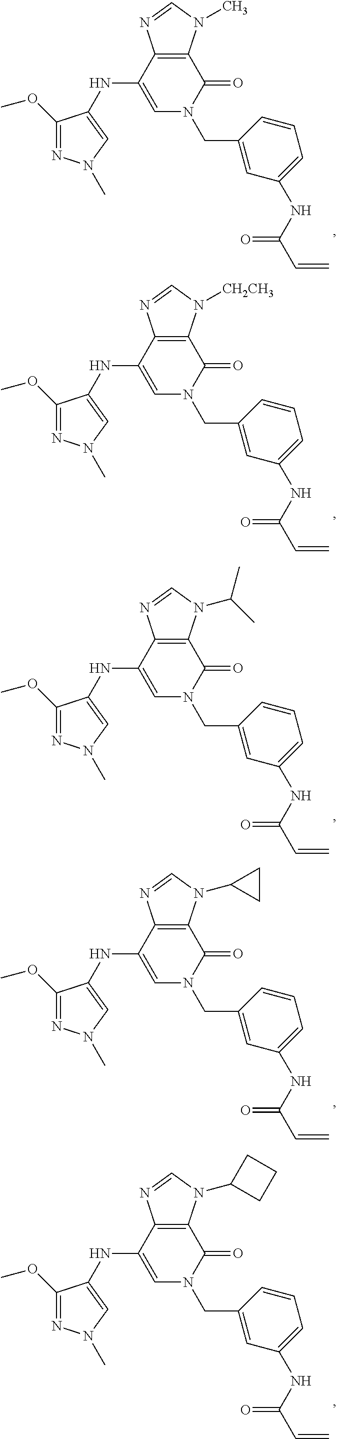

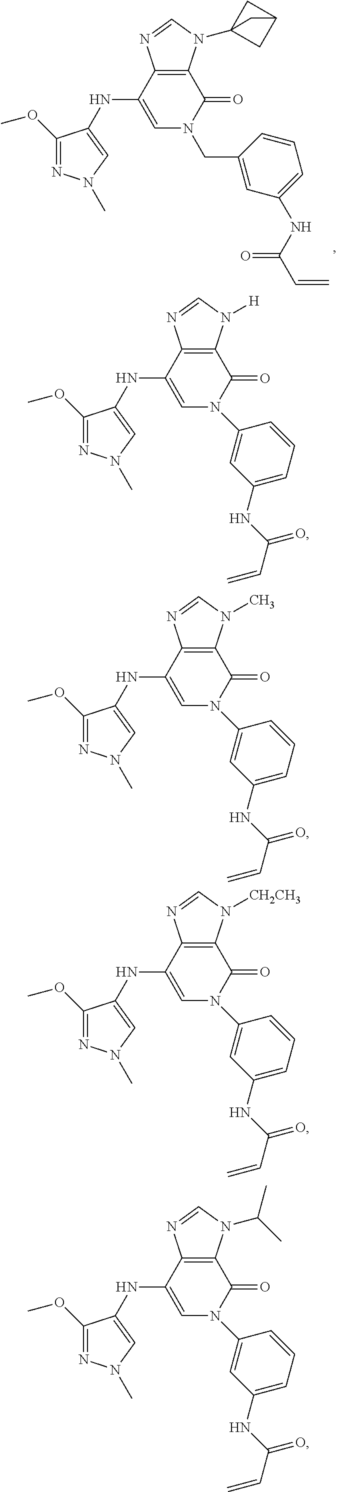

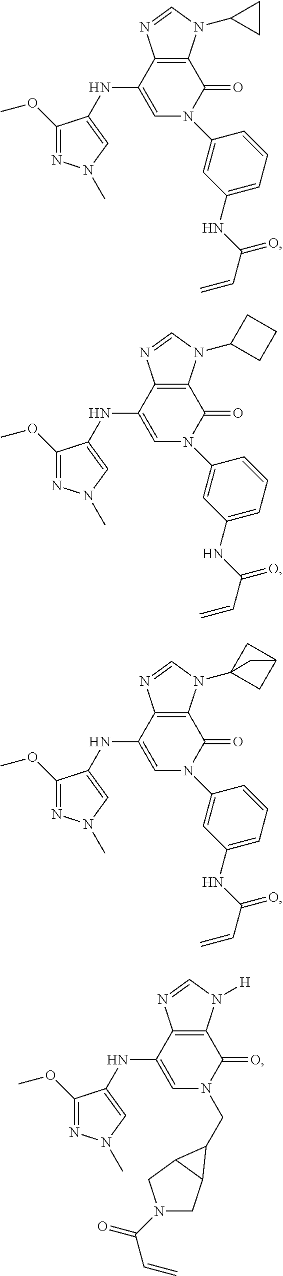

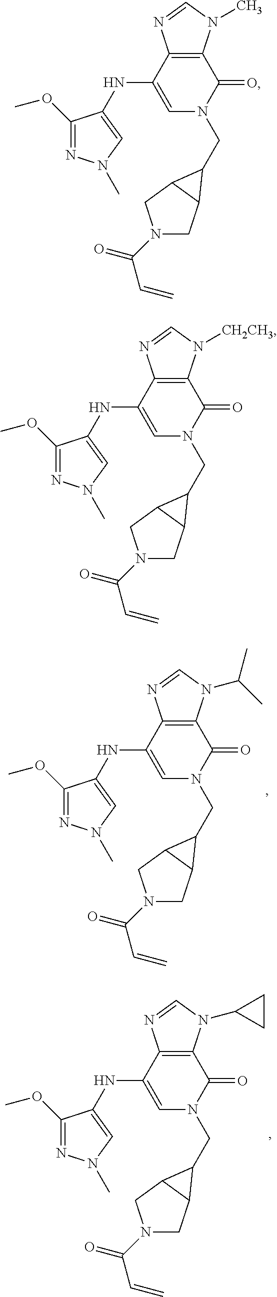

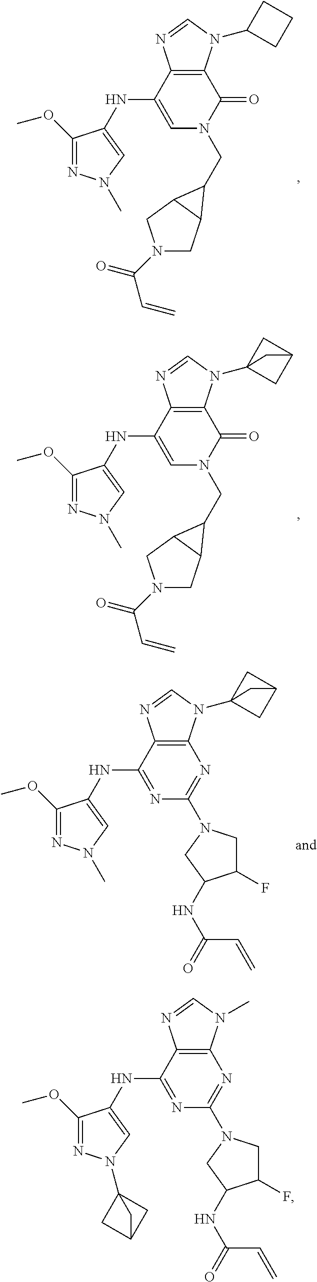

or a pharmaceutically acceptable salt of any of the foregoing.

In some embodiments, ring Z cannot be

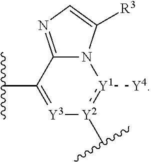

In other embodiments, ring Z cannot be

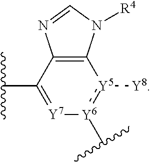

In some embodiments, when Ring Z is

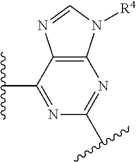

then R⁴ cannot be an unsubstituted C₁₋₄ alkyl, a C₁₋₄ alkyl substituted with hydroxy, an unsubstituted C₃-C₄ cycloalkyl or a C₃-C₄ cycloalkyl substituted with an unsubstituted C₁₋₄ alkyl. In some embodiments, ring Z cannot be

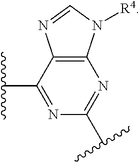

In some embodiments, R³ cannot be hydrogen. In some embodiments, R³ cannot be halogen. In still other embodiments, R³ cannot be an optionally substituted C₁₋₄ alkyl. In some embodiments, R³ cannot be an unsubstituted C₁₋₄ alkyl, such as methyl, ethyl, n-propyl, iso-propyl, n-butyl, iso-butyl and/or tert-butyl. In some embodiments, R⁴ cannot be hydrogen. In some embodiments, R⁴ cannot be halogen. In still other embodiments, R⁴ cannot be an optionally substituted C₁₋₄ alkyl. In some embodiments, R⁴ cannot be an unsubstituted C₁₋₄ alkyl, such as methyl, ethyl, n-propyl, iso-propyl, n-butyl, iso-butyl and/or tert-butyl. In some embodiments, R² cannot be an optionally substituted 5-membered heterocyclyl. For example, R² cannot be a substituted or unsubstituted pyrrolidinyl. In some embodiments, R² cannot be an optionally substituted heterocyclyl. In some embodiments, R² cannot be substituted with one or more of halogen (for example, fluoro) and an optionally substituted N-linked amido. In some embodiments, R² cannot be substituted with —NHC(═O)ethenyl. In some embodiments, R² cannot be

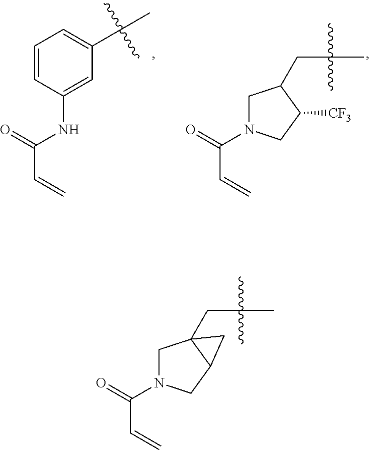 [SMILES: *c1cccc(NC(=O)C=C)c1 ; *CC1CN(C(=O)C=C)C[C@H]1C(F)(F)F ; *CC12CC1CN(C(=O)C=C)C2]

and/or

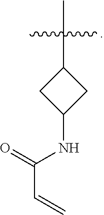 [SMILES: *C1CC(NC(=O)C=C)C1]

In some embodiments, ring Z cannot be

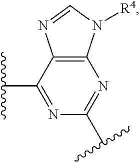

wherein R⁴ is an unsubstituted or substituted C₁₋₄ alkyl. In some embodiments, ring Z cannot be

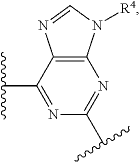

wherein R⁴ is an unsubstituted or substituted C₃₋₁₀ cycloalkyl. In some embodiments, R¹ cannot be an optionally substituted heteroaryl. As one example, R¹ cannot be an optionally substituted monocyclic heteroaryl. In some embodiments, R¹ cannot be pyrazolyl. In some embodiments, R¹ cannot be a substituted heteroaryl substituted with a substituent selected from an alkoxy (for example, methoxy), an optionally substituted C₁₋₄ alkyl and an optionally substituted C₁₋₄ alkynyl. In some embodiments, m cannot be 0. In some embodiments, m cannot be 0 when n is 0. In some embodiments, ring Z cannot be

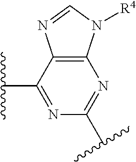

when m and n are each 0. In some embodiments, a compound of Formula (I), or a pharmaceutically acceptable salt thereof, cannot be a compound provided in PCT Publication No. WO 2015/075598. In some embodiments, a compound of Formula (I), or a pharmaceutically acceptable salt, cannot be selected from:

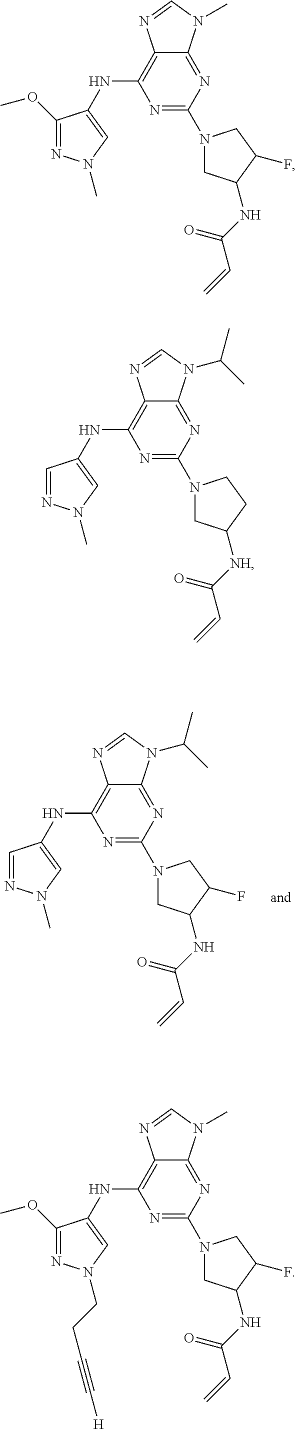

### Synthesis

Compounds of Formula (I), and those described herein may be prepared in various ways. Some compounds of Formula (I) can be obtained commercially and/or prepared utilizing known synthetic procedures. General synthetic routes to the compounds of Formula (I), and some examples of starting materials used to synthesize the compounds of Formula (I) are shown and described herein. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

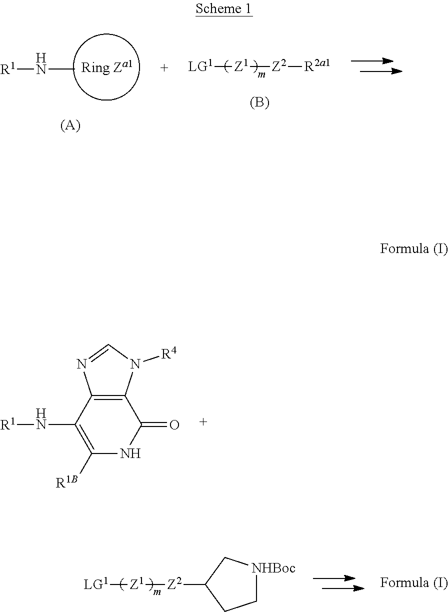

As shown in Scheme 1, compound (A) can be alkylated using compound (B) and one or more methods known to those skilled in the art. In Scheme 1, LG¹ can be a suitable leaving group, R¹, Z¹, Z² and m can be the same as described herein; Ring Z^{a1} can be the same as Ring Z as described herein except the nitrogen to which compound (B) attaches is —NH or a protected nitrogen; and R^{2a1} can be same as described herein or include a protected nitrogen. An example of Ring Z^{a1} and R^{2a1} with a protected nitrogen is shown in Scheme 1. Compound (A) and compound (B) can be coupled together via a nucleophilic substitution reaction or a Pd-catalyzed coupling reaction (such as a Buchwald-Hartwig reaction and/or a palladacycle coupling catalyst). If a protecting group is present on a nitrogen of R^{2a1}, the protecting group can be removed using methods known to those skilled in the art. An amide can be formed to provide a compound of Formula (I), or a pharmaceutically acceptable salt thereof. For example, the BOC group shown in Scheme 1 can be removed using an acid (such as HCl) and an acyl-halide (for example, acryloyl chloride) can be combined with the amine to form an amide.

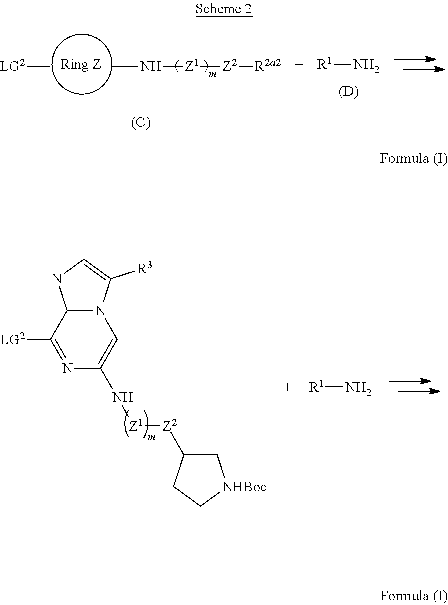

A compound of Formula (I), or a pharmaceutically acceptable salt thereof, can also be obtained as shown in Scheme 2. In Scheme 2, LG² can be a suitable leaving group, R¹, Z¹, Z², Ring Z and m can be the same as described herein; and R^{2a2} can be same as described herein or include a protected nitrogen. A more detailed example of compound (C) is also provided in Scheme 2. Compound (D) and compound (C) can be coupled together via a nucleophilic substitution reaction or a Pd-catalyzed cross-coupling reaction (for example, Buchwald-Hartwig reaction). If a protecting group is present on a nitrogen of R^{2a2}, the protecting group can be removed using methods known to those skilled in the art, and an amide can be formed to provide a compound of Formula (I), or a pharmaceutically acceptable salt thereof. As described previously, the BOC group can be removed using an acid and an acyl-halide can be combined with the amine to form an amide. Additional details regarding routes and materials are provided herein, such as in Schemes A-F.

Examples of suitable leaving groups and protecting groups are known to those skilled in the art and described herein. In some embodiments, LG¹ can be a halide, such as chloride. In some embodiments, LG² can be a halide, such as chloride, or alkyl-SO₂.

### Pharmaceutical Compositions

Some embodiments described herein relate to a pharmaceutical composition, that can include an effective amount of one or more compounds described herein (e.g., a compound of Formula (I), or a pharmaceutically acceptable salt thereof) and a pharmaceutically acceptable carrier, diluent, excipient or combination thereof.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, pulmonary, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory infection may be desirable.

As described herein, compounds of Formula (I), or a pharmaceutically acceptable salt thereof, can be administered by a variety of methods. In some of the methods described herein, administration can be by injection, infusion and/or intravenous administration over the course of 1 minute, 5 minutes, 10 minutes, 30 minutes, 1 hour, 2 hours, 6 hours, 12 hours, 24 hours or longer, or any intermediate time. Other methods described herein can include oral, intravenous and/or intraperitoneal administration to a subject in need thereof, for example, to a subject to treat a cancer described herein responsive to an EGFR inhibitor.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Use

Some embodiments described herein relate to a method for ameliorating and/or treating a cancer described herein that can include administering an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for ameliorating and/or treating a cancer described herein. Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for ameliorating and/or treating a cancer described herein.

Some embodiments described herein relate to a method for inhibiting replication of a malignant growth or a tumor that can include contacting the growth or the tumor with an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof), wherein the malignant growth or tumor is due to a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for inhibiting replication of a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for inhibiting replication of a malignant growth or a tumor, wherein the malignant growth or tumor is due to a cancer described herein.

Some embodiments described herein relate to a method for ameliorating or treating a cancer described herein that can include contacting a malignant growth or a tumor with an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for ameliorating or treating a cancer that can include contacting a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for ameliorating or treating a cancer that can include contacting a malignant growth or a tumor, wherein the malignant growth or tumor is due to a cancer described herein.

Some embodiments described herein relate to a method for inhibiting the activity of EGFR (for example, inhibiting the activity of EGFR with acquired EGFR T790M mutation, or wildtype EGFR and where EGFR is overexpressed or activated) that can include providing an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) to a sample that includes a cancer cell from a cancer described herein. Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for inhibiting the activity of EGFR (for example, inhibiting the activity of EGFR with acquired EGFR T790M mutation, or wildtype EGFR and where EGFR is overexpressed or activated). Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for inhibiting the activity of EGFR (for example, inhibiting the activity of EGFR with acquired EGFR T790M mutation, or wildtype EGFR and where EGFR is overexpressed or activated).

Some embodiments described herein relate to a method for ameliorating or treating a cancer described herein that can include inhibiting the activity of EGFR (for example, inhibiting the activity of EGFR with acquired EGFR T790M mutation, or wildtype EGFR and where EGFR is overexpressed or activated) using an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) in the manufacture of a medicament for ameliorating or treating a cancer described herein by inhibiting the activity of EGFR (for example, inhibiting the activity of EGFR with acquired EGFR T790M mutation, or wildtype EGFR and where EGFR is overexpressed or activated). Still other embodiments described herein relate to an effective amount of a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) for ameliorating or treating a cancer described herein by inhibiting the activity of EGFR (for example, inhibiting the activity of EGFR with acquired EGFR T790M mutation, or wildtype EGFR and where EGFR is overexpressed or activated).

Examples of suitable cancers include, but are not limited to: lung cancers (e.g., lung adenocarcinoma and non-small cell lung cancer), pancreatic cancers (e.g., pancreatic carcinoma such as, for example, exocrine pancreatic carcinoma), colon cancers (e.g., colorectal carcinomas, such as, for example, colon adenocarcinoma and colon adenoma), breast cancers, prostate cancers, head and neck cancers (e.g., squamous cell cancer of the head and neck), ovarian cancers, brain cancers (e.g., gliomas, such as glioma blastoma multiforme), and kidney carcinomas.

As described herein, a cancer can become resistant to one or more anti-cancer agents. In some embodiments, a compound described herein (for example, a compound of Formula (I), or a pharmaceutically acceptable salt thereof) or a pharmaceutical composition that includes of a compound described herein (for example, a compound of Formula (I), or a pharmaceutically acceptable salt thereof) can be used to treat and/or ameliorate a cancer that has become resistant to one or more anti-cancer agents (such as one or more EGFR inhibitors). Examples of anti-cancer agents that a subject may have developed resistance to include, but are not limited to, first generation EGFR inhibitors (such as gefitinib and erlotinib) and second generation EGFR inhibitors (for example, afatinib). In some embodiments, the cancer that has become resistant to one or more anti-cancer agents can be a cancer described herein.

Several known EGFR inhibitors can cause one or more undesirable side effects in the subject being treated. Two examples of these side effects are hyperglacemia and a rash. The rash can be characterized by mild scaling, pimples, roughness, a feeling of tightness, itching and burning. In some embodiments, a compound described herein (for example, a compound of Formula (I), or a pharmaceutically acceptable salt thereof, can decrease the number and/or severity of one or more side effects associated with a known EGFR inhibitor. In some embodiments, a compound of Formula (I), or a pharmaceutically acceptable salt thereof, results in a severity of a side effect (such as one of those described herein) that is 25% less than compared to the severity of the same side effect experienced by a subject receiving a known EGFR inhibitor. In some embodiments, a compound of Formula (I), or a pharmaceutically acceptable salt thereof, results in a number of side effects that is 25% less than compared to the number of side effects experienced by a subject receiving a known EGFR inhibitor. In some embodiments, a compound of Formula (I), or a pharmaceutically acceptable salt thereof, results in a severity of a side effect (such as one of those described herein) that is less in the range of about 10% to about 30% compared to the severity of the same side effect experienced by a subject receiving a known EGFR inhibitor. In some embodiments, a compound of Formula (I), or a pharmaceutically acceptable salt thereof, results in a number of side effects that is in the range of about 10% to about 30% less than compared to the number of side effects experienced by a subject receiving a known EGFR inhibitor.

The compound(s) of Formula (I), or a pharmaceutically acceptable salt thereof, that can be used can be any of the embodiments described in paragraphs [0081]-[0102].

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance, and may positively affect one or more symptoms or aspects of the disease while having effects on other aspects of the disease or on unrelated systems that may be considered undesirable.

The terms “therapeutically effective amount” and “effective amount” are used to indicate an amount of an active compound, or pharmaceutical agent, that elicits the biological or medicinal response indicated. For example, a therapeutically effective amount of compound can be the amount needed to treat, alleviate or ameliorate one or more symptoms or conditions of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer) a therapeutically effective amount is that amount that alleviates or eliminates cough, shortness of breath and/or pain. As another example, an effective amount, or a therapeutically effective amount of an EGFR inhibitor is the amount which results in the reduction in EGFR activity and/or phosphorylation. The reduction in EGFR activity are known to those skilled in the art and can be determined by the analysis of EGFR intrinsic kinase activity and downstream substrate phosphorylation.

The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

Various indicators for determining the effectiveness of a method for treating a cancer, are known to those skilled in the art. Example of suitable indicators include, but are not limited to, the reduction, alleviation or disappearance of one or more symptoms caused by the cancer, the reduction of tumor size, the elimination of the tumor, and/or long-term disease stabilization (growth arrest) of the tumor.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

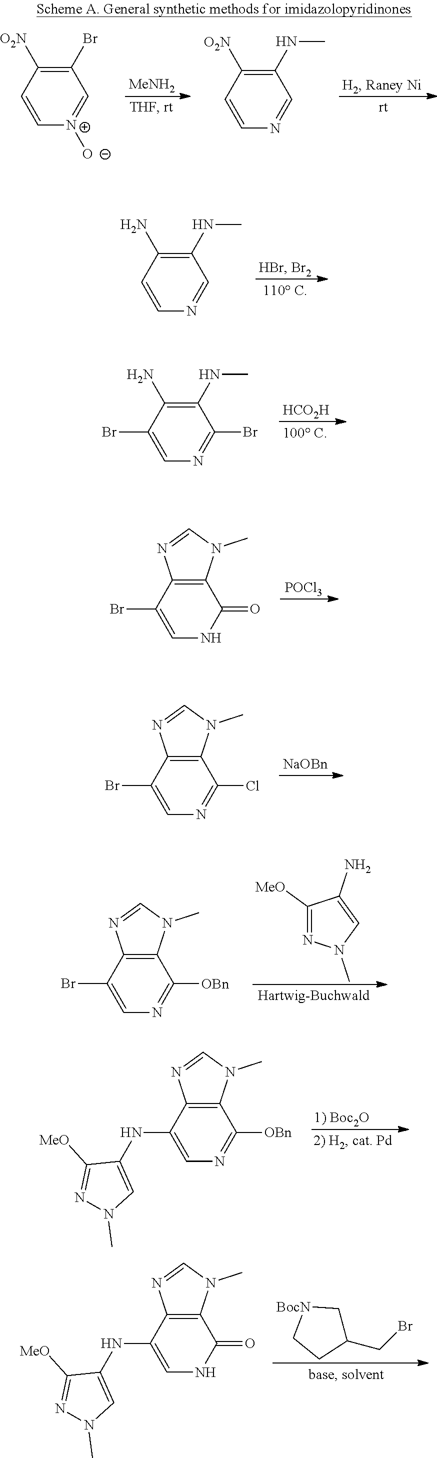

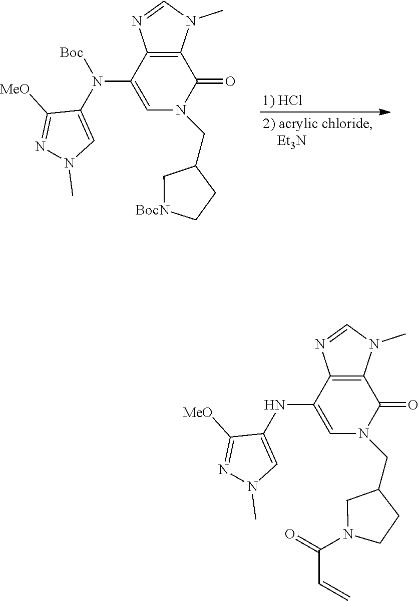 [SMILES: COc1nn(C)cc1N(C(=O)OC(C)(C)C)c1cn(CC2CCN(C(=O)OC(C)(C)C)C2)c(=O)c2c1ncn2C ; C=CC(=O)N1CCC(Cn2cc(Nc3cn(C)nc3OC)c3ncn(C)c3c2=O)C1]

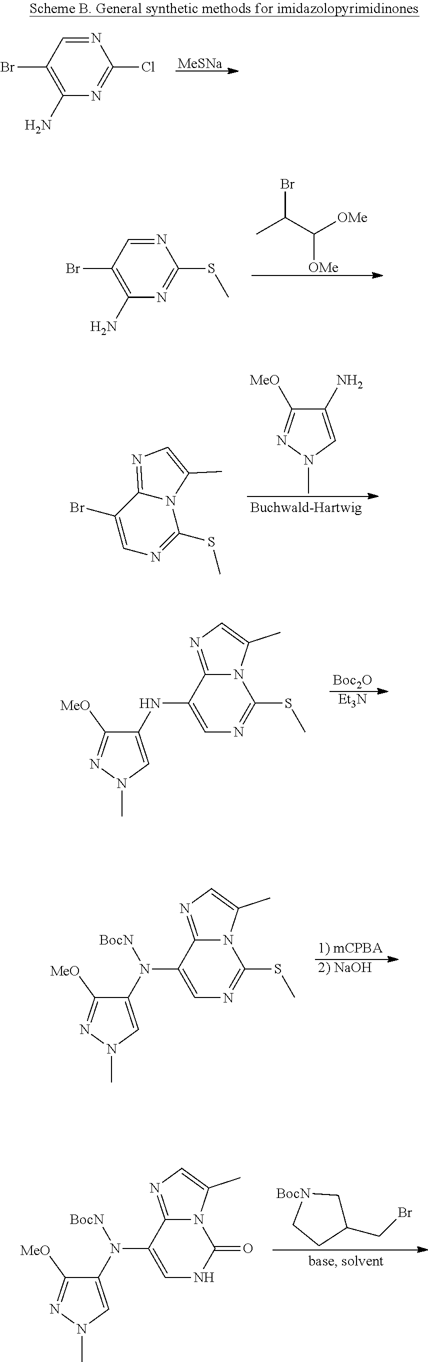

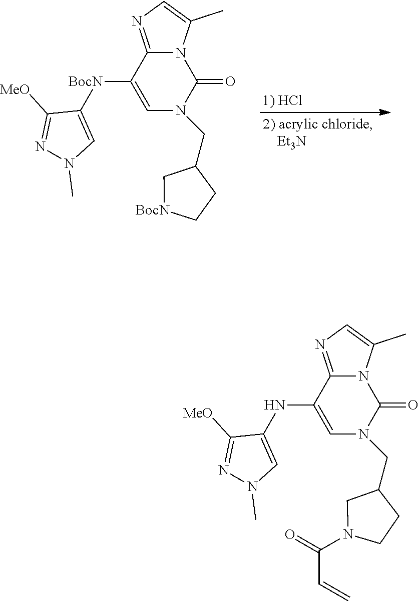 [SMILES: COc1nn(C)cc1N(C(=O)OC(C)(C)C)c1cn(CC2CCN(C(=O)OC(C)(C)C)C2)c(=O)n2c(C)cnc12 ; C=CC(=O)N1CCC(Cn2cc(Nc3cn(C)nc3OC)c3ncc(C)n3c2=O)C1]

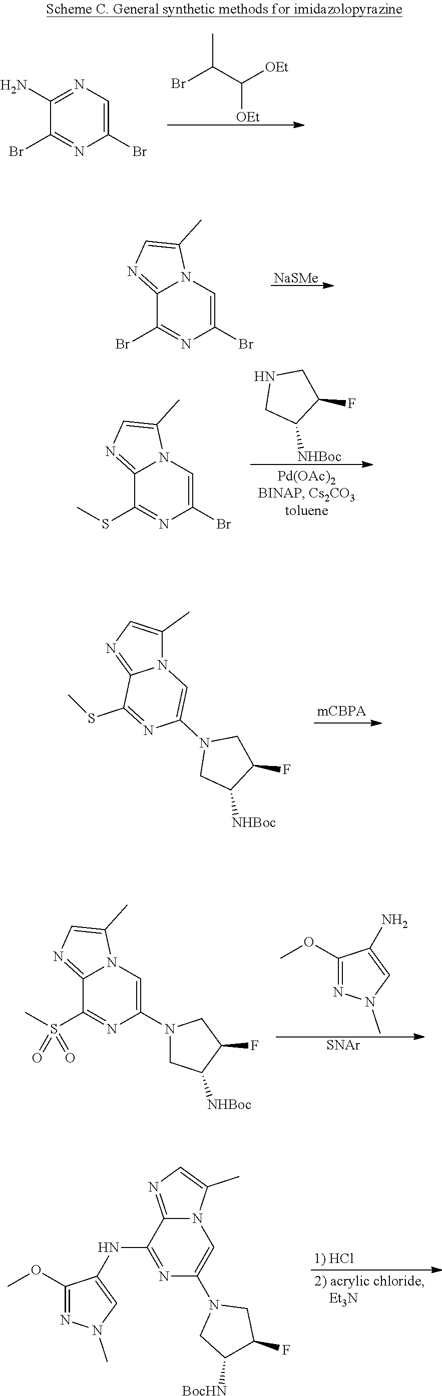

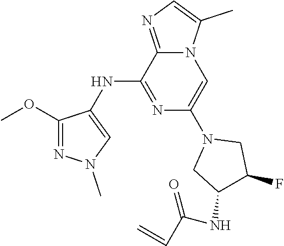 [SMILES: C=CC(=O)N[C@@H]1CN(c2cn3c(C)cnc3c(Nc3cn(C)nc3OC)n2)C[C@H]1F]

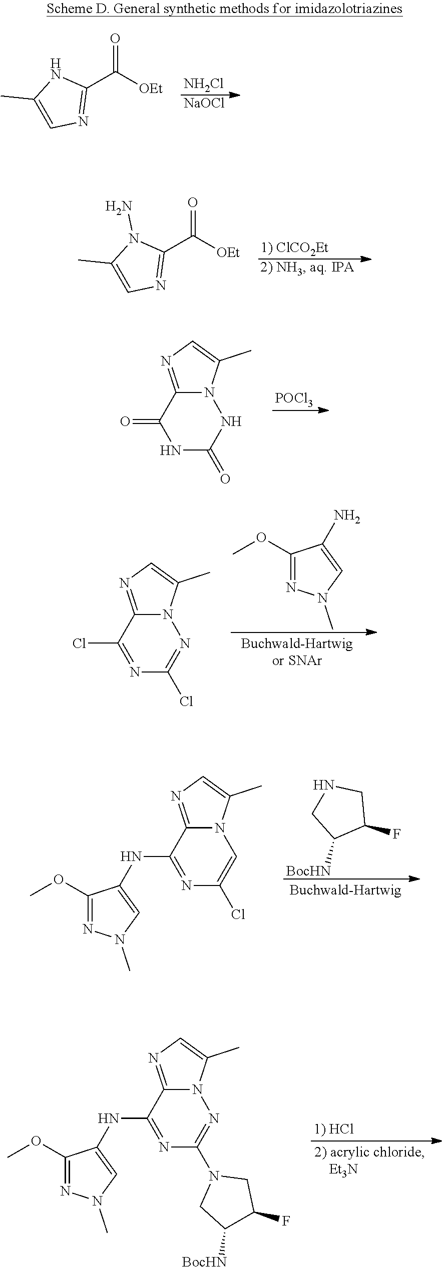

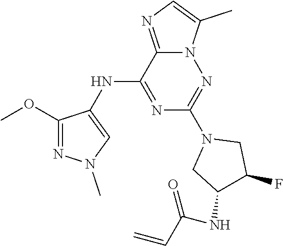 [SMILES: C=CC(=O)N[C@@H]1CN(c2nc(Nc3cn(C)nc3OC)c3ncc(C)n3n2)C[C@H]1F]

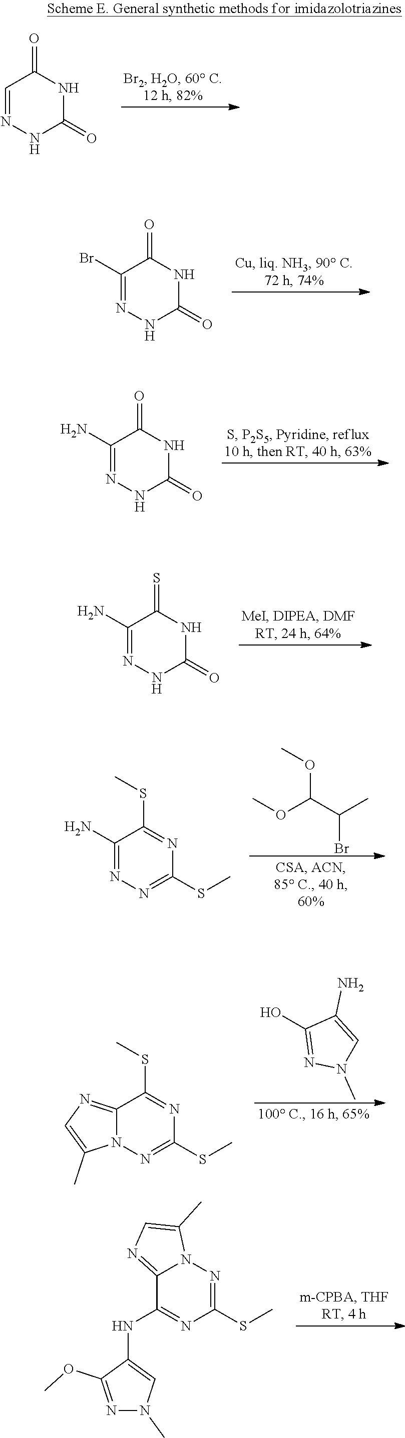

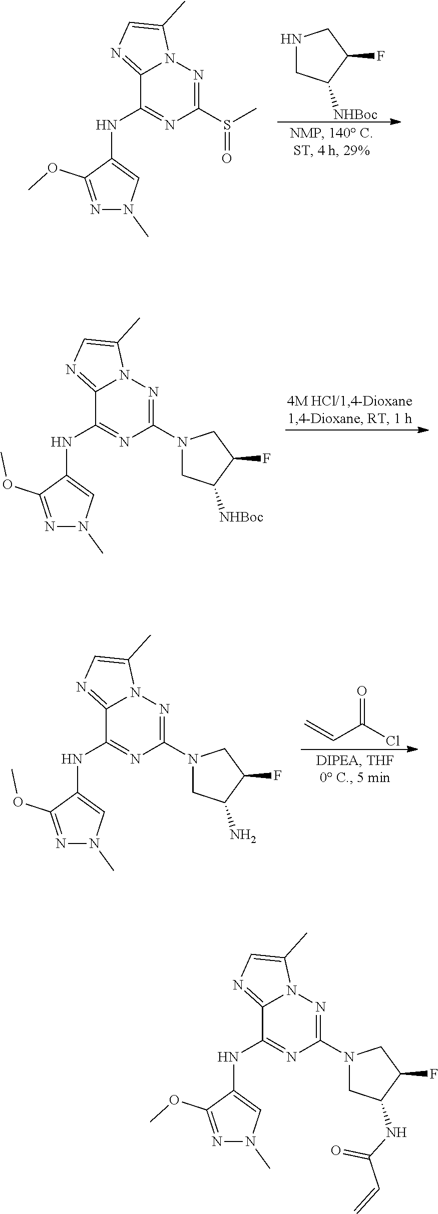

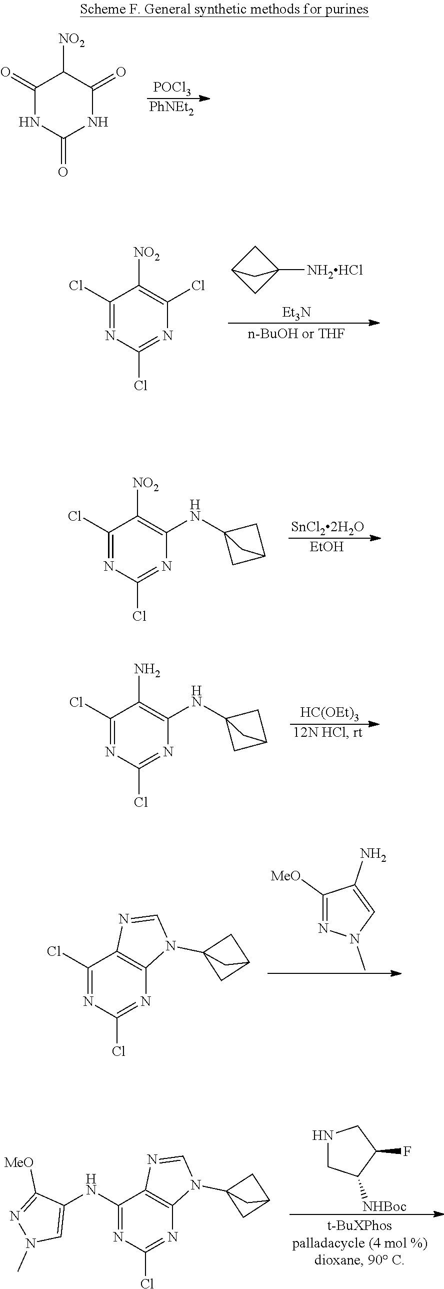

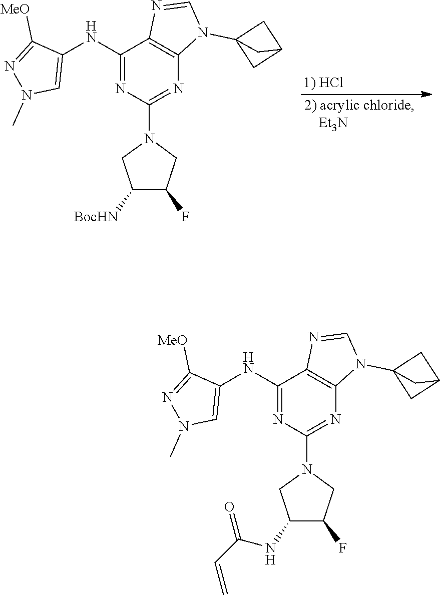 [SMILES: COc1nn(C)cc1Nc1nc(N2C[C@@H](F)[C@H](NC(=O)OC(C)(C)C)C2)nc2c1ncn2C12CC(C1)C2 ; C=CC(=O)N[C@@H]1CN(c2nc(Nc3cn(C)nc3OC)c3ncn(C45CC(C4)C5)c3n2)C[C@H]1F]

### Example 1

### N-((3R,4R)-4-fluoro-1-(4-((3-methoxy-1-methyl-1H-pyrazol-4-yl)amino)-7-methylimidazo[2,1-f][1,2,4]triazin-2-yl)pyrrolidin-3-yl)acrylamide

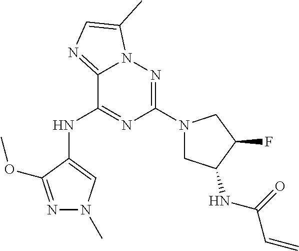 [SMILES: C=CC(=O)N[C@@H]1CN(c2nc(Nc3cn(C)nc3OC)c3ncc(C)n3n2)C[C@H]1F]

Step-1: To a stirred solution of ethyl 1H-imidazole-2-carboxylate (5.0 g, 35.6 mmol) in NMP (100 mL) was added potassium-tert-butoxide (1M in THF, 39.3 mL, 39.3 mmol) dropwise and the mixture was stirred for 15 mins. O-(4-nitrobenzoyl) hydroxylamine (7.14 g, 39.3 mmol) in NMP (50 mL) was added dropwise. The mixture was stirred at room temperature (RT) for 2 h. To the mixture, 2M HCl in diethyl ether (7 mL) was added at RT. After 10 mins, the mixture was diluted with diethyl ether (100 mL), and then stirred at RT for 30 mins. The precipitated solid was filtered and washed with diethyl ether (50 mL) to afford ethyl 1-amino-1H-imidazole-2-carboxylate dihydrochloride (8.12 g, 35.61 mmol, 99%) as an off-white solid. ¹H NMR (300 MHz, DMSO-d₆) δ 7.80 (s, 1H), 7.60 (s, 1H), 6.56 (br s, 2H), 4.40 (q, J=7.2 Hz, 2H), 1.30 (t, J=7.2 Hz, 3H); MS (ESI) m/z 156.02 [M+H]⁺.

Step-2: To a stirred solution of ethyl 1-amino-1H-imidazole-2-carboxylate dihydrochloride (8.1 g, 35.61 mmol) in THF (100 mL) and water (100 mL) were added sodium bicarbonate (21.37 g, 254.4 mmol) and ethylchloroformate (13.84 g, 127.5 mmol) at RT. and the mixture was stirred for 2 h. The mixture was diluted with ethyl acetate (2×100 mL). The organic layer was separated, washed with brine (50 mL), dried over sodium sulphate and concentrated under reduced pressure. The residue was purified by column chromatography (100-200 mesh, silica column) using 1% methanol in dichloromethane to afford ethyl 1-(bis(ethoxycarbonyl)amino)-1H-imidazole-2-carboxylate (7.8 g, 26.08 mmol, 74%) as a yellow syrup. ¹H NMR (300 MHz, CDCl₃) δ 7.21 (d, J=1.6 Hz, 1H), 7.11 (d, J=0.8 Hz, 1H), 4.40-4.27 (m, 6H), 1.39 (t, J=6.8 Hz, 3H), 1.27 (t, J=7.2 Hz, 6H); MS (ESI) m/z 300.85 [M+H]⁺.

Step-3: To a stirred solution of ethyl 1-(bis(ethoxycarbonyl)amino)-1H-imidazole-2-carboxylate (7.9 g, 26.42 mmol) in IPA (50 mL) was added NH₄OH solution (25%, 150 mL). The mixture was heated to 120° C. for 16 h in a steel bomb. The mixture was concentrated and triturated with methanol and diethyl ether (1:10 100 mL) to afford imidazo[2,1-f][1,2,4]triazine-2,4(1H,3H)-dione (3.1 g, 13.15 mmol, 77%) as an off white solid. ¹H NMR (300 MHz, DMSO-d₆) δ 7.20 (s, 1H), 7.05 (s, 1H), 5.40 (brs, 2H); MS (ESI) m/z 153.2 [M+H]⁺.

Step-4: To a stirred solution of imidazo[2,1-f][1,2,4]triazine-2,4(1H,3H)-dione (5.2 g, 34.2 mmol) in water (235 mL) was added NBS (4.26 g, 23.94 mmol) at 0° C., and the mixture was stirred at RT for 1 h. The mixture was filtered to remove insoluble materials, and the aqueous layer was washed with DCM (100 mL). The aqueous layer was then concentrated and distilled (azeotropic) with toluene (100 mL). The solid obtained was triturated with methanol (15 mL) to afford 7-bromoimidazo[2,1-f][1,2,4]triazine-2,4(1H,3H)-dione (5.0 g, 21.74 mmol, 64%) as an off white solid. ¹H NMR (300 MHz, DMSO-d₆) δ 7.01 (s, 1H), 5.4 (s, 2H). ¹H NMR (300 MHz, DMSO-d₆, D₂O exchange) δ 7.18 (s, 1H).

Step-5: To a stirred solution of 7-bromoimidazo[2,1-f][1,2,4]triazine-2,4(1H,3H)-dione (7.5 g, 32.60 mmol) in POCl₃ (125 mL) was added triethylamine hydrochloride (8.9 g, 65.206 mmol) at RT, and the mixture was then heated to 120° C. for 16 h under a sealed tube. The mixture was concentrated and distilled (azeotropic) with toluene (2×50 mL). The residue was diluted with ethyl acetate (2×250 mL) and poured into aq.NaHCO₃ solution (600 mL). The organic layer was separated, dried over sodium sulphate and concentrated under reduced pressure. The residue was purified by column chromatography (100-200 mesh, silica column) using 5% ethyl acetate in hexane to afford 7-bromo-2,4-dichloroimidazo[2,1-f][1,2,4]triazine (3 g, 11.27 mmol, 34%) as a pale yellow solid. ¹H NMR (400 MHz, CDCl₃) δ 8.01 (s, 1H).

Step-6: To a stirred solution of 7-bromo-2,4-dichloroimidazo[2,1-f][1,2,4]triazine (3.4 g, 12.73 mmol) and 3-methoxy-1-methyl-1H-pyrazol-4-amine (2.75 g, 21.64 mmol) in THF (170 mL) was added DIPEA (7.75 mL, 43.29 mmol). The mixture was stirred at RT for 1 h. The mixture was concentrated and water (100 mL) was added. The obtained solid was filtered and dried to afford 7-bromo-2-chloro-N-(3-methoxy-1-methyl-1H-pyrazol-4-yl)imidazo[2,1-f][1,2,4]triazin-4-amine (2.8 g, 7.84 mmol, 62%) as an off white solid. ¹H NMR (300 MHz, DMSO-d₆) δ 10.50 (br s, 1H), 7.80 (s, 1H), 7.79 (s, 1H), 3.81 (s, 3H), 3.73 (s, 3H); MS (ESI) m/z 357.86 [M+H]⁺ (for ⁷⁹Br) and MS (ESI) m/z 359.81 [M+H]⁺ (for ⁸¹Br).

Step-7: To a stirred and degassed solution of 7-bromo-2-chloro-N-(3-methoxy-1-methyl-1H-pyrazol-4-yl)imidazo[2,1-f][1,2,4]triazin-4-amine (900 mg, 2.52 mmol) in DMF (70 mL) was added cesium carbonate (4.09 g, 12.6 mmol), and the mixture was degassed for 10 mins. To this mixture was added trimethyl boroxine (1.4 mL, 10.08 mmol) and tris(dibenzylideneacetone)dipalladium(0) (230 mg, 0.252 mmol), followed by tricyclohexyl phosphine (71 mg, 0.252 mmol). The mixture was further degassing for 10 mins and then heated at 110° C. for 48 h. The mixture was cooled to RT and filtered through a celite pad. To the filtrate was added cold water, and the mixture was extracted with ethyl acetate (3×100 mL). The combined organic layers were washed with water (2×50 mL) and brine (1×100 mL), dried over sodium sulphate and concentrated. The resultant residue was purified by Reveleris C-18 reverse phase column using 55% acetonitrile in aqueous formic acid (0.1%) to afford 2-chloro-N-(3-methoxy-1-methyl-1H-pyrazol-4-yl)-7-methylimidazo[2,1-f][1,2,4]triazin-4-amine (300 mg, 1.023 mmol, 41%). ¹H NMR (300 MHz, DMSO-d₆) δ 10.18 (br s, 1H), 7.74 (s, 1H), 7.49 (s, 1H), 3.80 (s, 3H), 3.72 (s, 3H), 2.40 (s, 3H); MS (ESI) m/z 294.18 [M+H]⁺.

Step-8: To a stirred solution of 2-chloro-N-(3-methoxy-1-methyl-1H-pyrazol-4-yl)-7-methylimidazo[2,1-f][1,2,4]triazin-4-amine (400 mg, 1.365 mmol) in NMP (2 mL) was added tert-butyl 3-aminobicyclo[1.1.1]pentan-1-ylcarbamate (417 mg, 2.047 mmol), and the mixture was stirred at 140° C. for 3 h. To the mixture was added water (25 mL). The resulting solid was filtered and dried to afford tert-butyl ((3R,4R)-4-fluoro-1-(4-((3-methoxy-1-methyl-1H-pyrazol-4-yl)amino)-7-methylimidazo[2,1-f][1,2,4]triazin-2-yl)pyrrolidin-3-yl)carbamate (370 mg, 0.802 mmol, 59%) as an off white solid. ¹H NMR (300 MHz, DMSO-d₆) δ 9.00 (br s, 1H), 7.84 (s, 1H), 7.36 (br s, 1H), 7.22 (s, 1H), 5.08 (d, J=51.6 Hz, 1H), 4.13 (br d, J=5.1 Hz, 1H), 3.83 (s, 3H), 3.79-3.64 (m, 6H), 3.51 (br d, J=12.1 Hz, 1H), 2.33 (s, 3H), 1.39 (s, 9H); MS (ESI) m/z 462.00 [M+H]⁺.

Step-9: To a stirred solution of tert-butyl ((3R,4R)-4-fluoro-1-(4-((3-methoxy-1-methyl-1H-pyrazol-4-yl)amino)-7-methylimidazo[2,1-f][1,2,4]triazin-2-yl)pyrrolidin-3-yl)carbamate (220 mg, 0.477 mmol) in 1,4-dioxane (5 mL) was added 4M HCl in 1,4-dioxane (5 mL), and the mixture as stirred at RT for 1 h. The mixture was concentrated, dissolved with water (25 mL) and extracted with ethyl acetate (25 mL). The aqueous layer was basified with aq.NaHCO₃ and extracted with ethyl acetate (2×30 mL). The combined organic layer was washed with brine (50 mL), dried over sodium sulphate and concentrated under reduced pressure. The resultant residue was triturated with pentane (5 mL) to afford 2-((3R,4R)-3-amino-4-fluoropyrrolidin-1-yl)-N-(3-methoxy-1-methyl-1H-pyrazol-4-yl)-7-methylimidazo[2,1-f][1,2,4]triazin-4-amine (120 mg, 0.332 mmol, 70%) as an off white solid. ¹H NMR (300 MHz, DMSO-d₆) δ 7.85 (s, 1H), 7.21 (s, 1H), 4.94 (d, J=53.4 Hz, 1H), 3.84 (s, 3H), 3.72-3.56 (m, 7H), 3.40 (br d, J=10.3 Hz, 1H), 2.33 (s, 3H); MS (ESI) m/z 362.25 [M+H]⁺.

Step-10: To a stirred solution of 2-((3R,4R)-3-amino-4-fluoropyrrolidin-1-yl)-N-(3-methoxy-1-methyl-1H-pyrazol-4-yl)-7-methylimidazo[2,1-f][1,2,4]triazin-4-amine (0.12 g, 0.332 mmol) in THF:H₂O (1:1, 20 mL) was added DIPEA (0.115 mL, 0.664 mmol) followed by a solution of acryloyl chloride (0.021 mL, 0.394 mmol) in THF (1 mL) at 0° C. The mixture was stirred at 0° C. for 1 h. The mixture was diluted with water (30 mL) and extracted with ethyl acetate (2×50 mL). The combined organic layer was washed with brine (30 mL), dried over sodium sulphate and concentrated under reduced pressure. The resultant residue was purified by Reveleris C-18 reversed phase column using 55% acetonitrile in aqueous formic acid (0.1%) to afford N-((3R,4R)-4-fluoro-1-(4-((3-methoxy-1-methyl-1H-pyrazol-4-yl)amino)-7-methylimidazo[2,1-f][1,2,4]triazin-2-yl)pyrrolidin-3-yl)acrylamide (55 mg, 0.022 mmol, 40%) as an off white solid. ¹H NMR (300 MHz, DMSO-d₆) δ 9.04 (br s, 1H), 8.45 (br d, J=6.6 Hz, 1H), 7.85 (s, 1H), 7.23 (s, 1H), 6.35-6.04 (m, 2H), 5.63 (dd, J=2.9, 9.2 Hz, 1H), 5.13 (d, J=53.4 Hz, 1H), 4.58-4.40 (m, 1H), 3.88-3.55 (m, 10H), 2.34 (s, 3H); MS (ESI) m/z 416.32 [M+H]⁺.

### Example 2

### N-((3R,4R)-4-fluoro-1-(4-((3-methoxy-1-((S)-1-methylpyrrolidin-3-yl)-1H-pyrazol-4-yl)amino)-7-methylimidazo[2,1-f][1,2,4]triazin-2-yl)pyrrolidin-3-yl)acrylamide

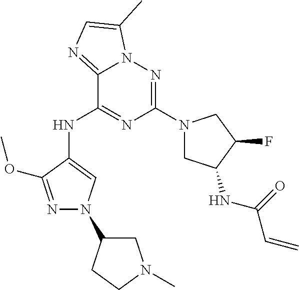

Step-1: (S)-7-bromo-2-chloro-N-(3-methoxy-1-(1-methylpyrrolidin-3-yl)-1H-pyrazol-4-yl)imidazo[2,1-f][1,2,4]triazin-4-amine was synthesized by following the same procedure as described in Example 1, and using (S)-3-methoxy-1-(1-methylpyrrolidin-3-yl)-1H-pyrazol-4-amine in step 6 to afford (S)-7-bromo-2-chloro-N-(3-methoxy-1-(1-methylpyrrolidin-3-yl)-1H-pyrazol-4-yl)imidazo[2,1-f][1,2,4]triazin-4-amine (50% yield). ¹H NMR (300 MHz, DMSO-d₆) δ 10.45 (br s, 1H), 7.90 (s, 1H), 7.80 (s, 1H), 4.68-4.65 (m, 1H), 3.81 (s, 3H), 2.80-2.71 (m, 3H), 2.43-2.41 (m, 1H), 2.35-2.32 (m, 4H), 2.11-2.01 (m, 1H); MS (ESI) m/z 427.11 [M+H]⁺ for ⁷⁹Br.

Step-2: To a mixture of (S)-7-bromo-2-chloro-N-(3-methoxy-1-(1-methylpyrrolidin-3-yl)-1H-pyrazol-4-yl)imidazo[2,1-f][1,2,4]triazin-4-amine (100 mg, 0.234 mmol), Palladium(II) acetate in ionic liquid on silica (5.25 mg, 0.023 mmol), in dioxane (2338 yl) (degassed) was added trimethylboroxin (176 mg, 1.403 mmol) in degassed dioxane (0.2 mL) followed by aq. cesium carbonate (234 μl, 0.351 mmol, 1.5 M) degassed with argon, and then 1,1′-Bis(di-i-propylphosphino)ferrocene (19.56 mg, 0.047 mmol). The vial was sealed and degassed with argon 3-4 times. The mixture was heated at 90° C. for 16 h. The mixture was filtered through a celite pad and the celite pad was washed with methanol. The filtrate was collected, concentrated and purified by reverse phase HPLC using 10-80% acetonitrile (contains 0.1% formic acid) in water (contains 0.1% formic acid) to afford (S)-2-chloro-N-(3-methoxy-1-(1-methylpyrrolidin-3-yl)-1H-pyrazol-4-yl)-7-methylimidazo[2,1-f][1,2,4]triazin-4-amine (32 mg, 0.088 mmol, 37%) after lyophilization. ¹H NMR (400 MHz, CDCl₃) δ 8.42 (br s, 1H), 8.15 (s, 1H), 7.35 (s, 1H), 4.93-4.91 (m, 1H), 3.98 (s, 3H), 3.86-3.82 (m, 1H), 3.46-3.41 (m, 1H), 3.25-3.18 (m, 2H), 2.79 (s, 3H), 2.56-2.53 (m, 1H), 2.49 (s, 3H), 2.32-2.30 (m, 1H); MS (ESI) m/z 363.10 [M+H]⁺.

Step-3: To a solution of (S)-2-chloro-N-(3-methoxy-1-(1-methylpyrrolidin-3-yl)-1H-pyrazol-4-yl)-7-methylimidazo[2,1-f][1,2,4]triazin-4-amine (50 mg, 0.138 mmol) in NMP (689 yl) was added tert-butyl ((3S,4S)-4-fluoropyrrolidin-3-yl)carbamate (84 mg, 0.413 mmol). The mixture was heated in a sealed tube at 140° C. for 3 h. The mixture was cooled to RT and purified by reverse phase HPLC using 10-80% acetonitrile (contains 0.1% formic acid) in water (contains 0.1% formic acid) to afford tert-butyl ((3S,4S)-4-fluoro-1-(4-((3-methoxy-1-((S)-1-methylpyrrolidin-3-yl)-1H-pyrazol-4-yl)amino)-7-methylimidazo[2,1-f][1,2,4]triazin-2-yl)pyrrolidin-3-yl)carbamate (27 mg, 0.051 mmol, 36.9%). ¹H NMR (400 MHz, DMSO-d₆) δ 9.01 (s, 1H), 8.13 (s, 1H), 7.38 (s, 1H), 7.20 (s, 1H), 5.17-5.04 (d, 1H), 4.73-4.72 (m, 1H), 4.16-4.14 (m, 1H), 3.84 (s, 3H), 3.80-3.51 (m, 4H), 2.83-2.76 (m, 2H), 2.66-2.62 (m, 1H), 2.38-2.36 (m, 5H), 2.33 (s, 3H), 1.98-1.95 (m, 1H), 1.37 (s, 9H); MS (ESI) m/z 531.30 [M+H]⁺.

Step-4: A solution of tert-butyl ((3S,4S)-4-fluoro-1-(4-((3-methoxy-1-((S)-1-methylpyrrolidin-3-yl)-1H-pyrazol-4-yl)amino)-7-methylimidazo[2,1-f][1,2,4]triazin-2-yl)pyrrolidin-3-yl)carbamate (24 mg, 0.045 mmol) in 4M HCl in dioxane (1.5 mL, 6.00 mmol) was stirred at RT for 1 h. The solvents were evaporated, and the residue was sonicated with ether. The mixture was filtered, and the precipitate was collected to afford 2-((3S,4S)-3-amino-4-fluoropyrrolidin-1-yl)-N-(3-methoxy-1-((S)-1-methylpyrrolidin-3-yl)-1H-pyrazol-4-yl)-7-methylimidazo[2,1-f][1,2,4]triazin-4-amine (16 mg, 0.037 mmol, 76%) as a hydrochloride salt, which was used for next step without further purification. MS (ESI) m/z 431.20 [M+H]⁺.

Step-5: To a solution of 2-((3S,4S)-3-amino-4-fluoropyrrolidin-1-yl)-N-(3-methoxy-1-((S)-1-methylpyrrolidin-3-yl)-1H-pyrazol-4-yl)-7-methylimidazo[2,1-f][1,2,4]triazin-4-amine (32 mg, 0.074 mmol) in THF (2.47 mL) at 0° C. was added N,N-Diisopropylethylamine (78 μl, 0.446 mmol) under N₂ atmosphere. To this mixture was added acryloyl chloride (6.73 mg, 0.074 mmol) in THF (0.2 mL THF). The mixture was stirred at 0° C. for 10 mins. The mixture was diluted with dichloromethane:water. The organic layer was separated, dried over Na₂SO₄, concentrated and purified by reverse phase HPLC using 10-80% acetonitrile (contains 0.1% formic acid) in water (contains 0.1% formic acid) to afford N-((3S,4S)-4-fluoro-1-(4-((3-methoxy-1-((S)-1-methylpyrrolidin-3-yl)-1H-pyrazol-4-yl)amino)-7-methylimidazo[2,1-f][1,2,4]triazin-2-yl)pyrrolidin-3-yl)acrylamide (10 mg, 0.021 mmol, 28%). ¹H NMR (400 MHz, DMSO-d₆) δ 9.07 (s, 1H), 8.47-8.45 (m, 1H), 8.13 (s, 1H), 7.23 (s, 1H), 6.21-6.15 (m, 2H), 5.64-5.61 (m, 1H), 5.21 (d, 1H), 4.70 (brs, 1H), 4.50-4.40 (m, 1H), 3.84 (s, 3H), 3.81-3.61 (m, 4H), 2.82-2.75 (m, 2H), 2.65-2.64 (m, 1H), 2.36-2.26 (m, 8H), 1.98-1.95 (m, 1H); MS (ESI) m/z 485.30 [M+H]⁺.

### Intermediate 1

### 7-methyl-2,4-bis(methylthio)imidazo[2,1-f][1,2,4]triazine

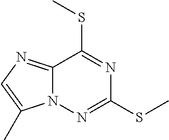

To a stirred solution of 3,5-bis(methylthio)-1,2,4-triazin-6-amine (10.0 g, 53.19 mmol) and 2-bromo-1,1-dimethoxypropane (19.6 g, 319.14 mmol, 2 times added), in CH₃CN (120 mL) was added (+/−)-camphor-10-sulfonic acid (3.70 gm, 15.95 mmol) and MS-4 Å (2 g). and the mixture was heated at 85° C. for 40 h. The mixture was cooled to RT and concentrated under reduced pressure to reduce the volume to 30 mL. The obtained solid was filtered and washed with CH₃CN (10 mL). The solid was dissolved in 20% MeOH in CH₂Cl₂ and filtered, and the filtrate was concentrated under reduced pressure to afford 7-methyl-2,4-bis(methylthio)imidazo[1,2-f][1,2,4]triazine as a pale brown solid (7 g, 58%). ¹H NMR (300 MHz, DMSO-d₆) δ 7.59 (br s, 1H), 2.63 (s, 3H), 2.60 (s, 3H), 2.48 (s, 3H); MS (ESI) m/z 227.12 [M+H]⁺.

### Example 3

### N-((3R,4R)-4-fluoro-1-(4-((3-methoxy-1-((S)-1-methylpyrrolidin-3-yl)-H-pyrazol-4-yl)amino)-7-methylimidazo[2,1-f][1,2,4]triazin-2-yl)pyrrolidin-3-yl)acrylamide

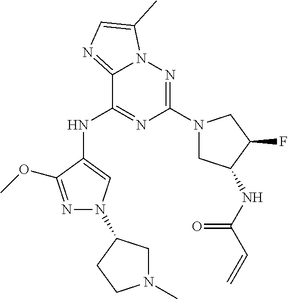 [SMILES: C=CC(=O)N[C@@H]1CN(c2nc(Nc3cn([C@H]4CCN(C)C4)nc3OC)c3ncc(C)n3n2)C[C@H]1F]

Step-1: To a stirred solution of DIAD (25.42 g, 125.8 mmol) in THF (200 mL) was added PPh₃ (33 g, 125.8 mmol) portionwise at 0° C., and the mixture was stirred at 0° C. for 5 mins. To this mixture was added 3-methoxy-4-nitro-1H-pyrazole (10 g, 69.93 mmol) in THF (300 mL) portionwise at 0° C. followed by a solution of (R)-1-methylpyrrolidin-3-ol (7.78 g, 76.92 mmol) in THF (100 mL) at 0° C. The mixture was stirred at RT for 16 h. The mixture was concentrated under reduced pressure, and the residue was purified by column chromatography on SiO₂ (5-10% MeOH in CH₂Cl₂) to afford (S)-3-methoxy-1-(1-methylpyrrolidin-3-yl)-4-nitro-1H-pyrazole as a pale yellow solid (13 g, 82%). ¹H NMR (300 MHz, DMSO-d₆) δ 8.72 (s, 1H), 4.82-4.72 (m, 1H), 3.94 (s, 3H), 2.89-2.70 (m, 3H), 2.45-2.25 (m, 5H), 2.18-2.05 (m, 1H); MS (ESI) m/z 227.03 [M+H]⁺.

Step-2: To a stirred solution of (S)-3-methoxy-1-(1-methylpyrrolidin-3-yl)-4-nitro-1H-pyrazole (5 g, 22.12 mmol) in MeOH (150 mL) was added Pd/C (10% wet; 2.5 g), and the mixture was stirred at RT under a hydrogen balloon (1 atm) for 3 h. The mixture was filtered through a pad of celite, and the filtrate was evaporated to afford (S)-3-methoxy-1-(1-methylpyrrolidin-3-yl)-1H-pyrazol-4-amine as a gummy pale brown liquid (3.46 g, 80%). ¹H NMR (300 MHz, DMSO-d₆) δ 6.99 (s, 1H), 4.55-4.48 (m, 1H), 3.74 (s, 3H), 3.65-3.45 (bs, 2H), 2.73-2.50 (m, 3H), 2.47-2.35 (m, 1H), 2.25-2.13 (m, 4H), 1.93-1.85 (m, 1H).

Step-3: A mixture of 7-methyl-2,4-bis(methylthio)imidazo[2,1-f][1,2,4]triazine (3.0 g, 13.27 mmol, intermediate 1) and (S)-3-methoxy-1-(1-methylpyrrolidin-3-yl)-1H-pyrazol-4-amine (3.33 g, 17.25 mmol) was heated to 100° C. for 20 h. The mixture was purified by column chromatography on SiO₂ (10-15% methanol in DCM) to afford (S)—N-(3-methoxy-1-(1-methylpyrrolidin-3-yl)-1H-pyrazol-4-yl)-7-methyl-2-(methylthio)imidazo[2,1-f][1,2,4]triazin-4-amine as a pale yellow solid (2.0 g, 40%). ¹H NMR (300 MHz, CD₃OD) δ 8.18 (s, 1H), 7.31 (s, 1H), 5.19-5.08 (m, 1H), 4.02 (s, 3H), 3.80-3.66 (m, 3H), 3.50-3.34 (m, 1H), 3.01 (s, 3H), 2.69-2.59 (m, 1H), 2.57 (s, 3H), 2.46 (s, 3H), 2.45-2.33 (m, 1H); MS (ESI) m/z 375.19 [M+H]⁺.

Step-4: To a stirred solution of (S)—N-(3-methoxy-1-(1-methylpyrrolidin-3-yl)-1H-pyrazol-4-yl)-7-methyl-2-(methylthio)imidazo[2,1-f][1,2,4]triazin-4-amine (2.0 g, 5.34 mmol) in acetone: water (2:1, 75 mL) was added oxone (3.6 g, 5.874 mmol), and the mixture was stirred at 0° C. for 1 h. Acetone was removed under reduced pressure. The reaction was quenched with aq.NaHCO₃ solution (50 mL) and extracted with EtOAc (2×50 mL). The combined organic layer was washed with brine (30 mL), dried over Na₂SO₄ and concentrated under reduced pressure. The resultant residue was purified by column chromatography on SiO₂ (12-16% MeOH in DCM) to afford N-(3-methoxy-1-((S)-1-methylpyrrolidin-3-yl)-1H-pyrazol-4-yl)-7-methyl-2-(methylsulfinyl)imidazo[2,1-f][1,2,4]triazin-4-amine as an off-white solid (700 mg, 34%). ¹H NMR (300 MHz, CD₃OD) δ 8.33 (s, 1H), 7.48 (s, 1H), 4.93-4.90 (m, 1H), 4.01 (s, 3H), 3.51-3.45 (m, 1H), 3.24-3.20 (m, 1H), 3.10-3.02 (m, 4H), 2.88-2.82 (m, 1H), 2.54-2.41 (m, 7H), 2.32-2.22 (m, 1H); MS (ESI) m/z 391.33 [M+H]⁺.

Step-5: To a stirred solution of N-(3-methoxy-1-((S)-1-methylpyrrolidin-3-yl)-1H-pyrazol-4-yl)-7-methyl-2-(methylsulfinyl)imidazo[2,1-f][1,2,4]triazin-4-amine (700 mg, 1.79 mmol) in NMP (1 mL) was added tert-butyl (3R,4R)-4-fluoropyrrolidin-3-ylcarbamate (548 mg, 2.68 mmol), and the mixture was stirred at 140° C. for 3 h in a sealed tube. To the mixture was added H₂O (25 mL), and the mixture was then extracted with EtOAc (2×50 mL). The combined organic layer was washed with brine (30 mL), dried over Na₂SO₄ and concentrated under reduced pressure. The resultant residue was purified by chromatography on SiO₂ (8-12% MeOH in DCM) to afford of tert-butyl ((3R,4R)-4-fluoro-1-(4-((3-methoxy-1-((S)-1-methylpyrrolidin-3-yl)-1H-pyrazol-4-yl)amino)-7-methylimidazo[2,1-f][1,2,4]triazin-2-yl)pyrrolidin-3-yl)carbamate as a pale brown solid (250 mg 26%). MS (ESI) m/z 531.37 [M+H]⁺.

Step-6: To a stirred solution of tert-butyl ((3R,4R)-4-fluoro-1-(4-((3-methoxy-1-((S)-1-methylpyrrolidin-3-yl)-1H-pyrazol-4-yl)amino)-7-methylimidazo[2,1-f][1,2,4]triazin-2-yl)pyrrolidin-3-yl)carbamate (250 mg, 0.471 mmol) in 1,4-dioxane (10 mL) was added 4M HCl in 1,4-dioxane (10 mL) at 0° C., and the mixture was stirred at RT for 1 h. The mixture was concentrated, dissolved with water (25 mL) and washed with EtOAc (15 mL). The aqueous layer was basified with aq.NaHCO₃ solution and extracted with EtOAc (2×30 mL). The combined organic layer was washed with brine (50 mL), dried over Na₂SO₄ and concentrated under reduced pressure. The resultant residue was triturated with pentane (3 mL) to afford 2-((3R,4R)-3-amino-4-fluoropyrrolidin-1-yl)-N-(3-methoxy-1-((S)-1-methylpyrrolidin-3-yl)-1H-pyrazol-4-yl)-7-methylimidazo[2,1-f][1,2,4]triazin-4-amine as an off-white solid (130 mg, 64%). MS (ESI) m/z 431.37 [M+H]⁺.

Step-7: To a stirred solution of 2-((3R,4R)-3-amino-4-fluoropyrrolidin-1-yl)-N-(3-methoxy-1-((S)-1-methylpyrrolidin-3-yl)-1H-pyrazol-4-yl)-7-methylimidazo[2,1-f][1,2,4]triazin-4-amine (0.13 g, 0.302 mmol) in THF (5 mL) was added DIPEA (0.158 mL, 0.906 mmol) followed by a solution of acryloyl chloride (24.5 mg, 0.272 mmol) in THF (1 mL) at 0° C. The mixture was stirred at 0° C. for 5 mins. H₂O (30 mL) was added to the mixture, and the mixture was then extracted with EtOAc (2×50 mL). The combined organic layer was washed with brine (30 mL), dried over Na₂SO₄ and concentrated under reduced pressure. The resultant residue was purified by triturating with CH₃CN (0.5 mL) to afford N-((3R,4R)-4-fluoro-1-(4-((3-methoxy-1-((S)-1-methylpyrrolidin-3-yl)-1H-pyrazol-4-yl)amino)-7-methylimidazo[2,1-f][1,2,4]triazin-2-yl)pyrrolidin-3-yl)acrylamide as an off-white solid (30 mg, 21%). ¹H NMR (400 MHz, DMSO-d₆) δ 9.08 (br s, 1H), 8.46 (d, J=6.8 Hz, 1H), 8.12 (s, 1H), 7.24 (s, 1H), 6.25-6.10 (m, 2H), 5.63 (dd, J=2.4, 9.2 Hz, 1H), 5.14 (d, J=50.8 Hz, 1H), 4.79-4.71 (m, 1H), 4.52-4.44 (m, 1H), 3.85 (s, 3H), 3.80-3.60 (m, 4H), 2.90-2.80 (m, 3H), 2.35-2.31 (m, 8H), 2.01-1.92 (m, 1H); MS (ESI) m/z 484.85 [M+H]⁺.

### Example 4

### N-((3R,4R)-4-fluoro-1-(4-((3-methoxy-1-(1-methylpiperidin-4-yl)-1H-pyrazol-4-yl)amino)-7-methylimidazo[2,1-f][1,2,4]triazin-2-yl)pyrrolidin-3-yl)acrylamide

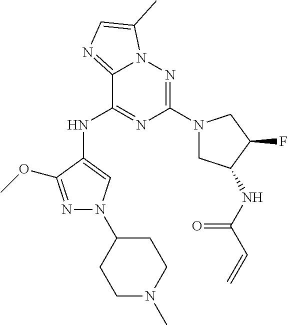 [SMILES: C=CC(=O)N[C@@H]1CN(c2nc(Nc3cn(C4CCN(C)CC4)nc3OC)c3ncc(C)n3n2)C[C@H]1F]

Step-1: To a stirred solution of DIAD (35.3 g, 174.8 mmol) in THF (200 mL) was added PPh₃ (46 g, 174.8 mmol) portionwise at 0° C. and stirred at 0° C. for 5 mins. To this mixture was added 3-methoxy-4-nitro-1H-pyrazole (10 g, 69.93 mmol) in THF (300 mL) dropwise at 0° C., followed by a solution of 1-methylpyrrolidin-3-ol (12.1 g, 104.9 mmol) in THF (100 mL) at 0° C. The mixture was stirred at RT for 16 h. The mixture was concentrated under reduced pressure, and the resultant residue was purified by column chromatography on SiO₂ (5-10% MeOH in CH₂Cl₂) to afford 4-(3-methoxy-4-nitro-1H-pyrazol-1-yl)-1-methylpiperidine as a pale yellow solid (7 g, 41%). ¹H NMR (300 MHz, DMSO-d₆) δ 8.73 (s, 1H), 4.10-3.98 (m, 1H), 3.94 (s, 3H), 2.90-2.80 (m, 2H), 2.19 (s, 3H), 2.07-1.87 (m, 6H); MS (ESI) m/z 241.2 [M+H]⁺.

Step-2: To a stirred solution of 4-(3-methoxy-4-nitro-1H-pyrazol-1-yl)-1-methylpiperidine (5 g, 20.83 mmol) in MeOH (150 mL) was added Pd/C (10%, 2.5 g), and the mixture was stirred at RT under hydrogen (1 atm) for 3 h. The mixture was filtered through a pad of celite, and the obtained filtrate was evaporated to afford 3-methoxy-1-(1-methylpiperidin-4-yl)-1H-pyrazol-4-amine as a pale brown gummy liquid (3.5 g, 80%). ¹H NMR (400 MHz, DMSO-d₆) δ 6.98 (s, 1H), 3.78-3.67 (m, 4H), 3.60-3.30 (br s, 2H), 2.87-2.77 (m, 2H), 2.20 (s, 3H), 2.07-1.74 (m, 6H); MS (ESI) m/z 211.26 [M+H]⁺.

Step-3: A mixture of 3-methoxy-1-(1-methylpiperidin-4-yl)-1H-pyrazol-4-amine (2.8 g, 13.33 mmol) and 7-methyl-2,4-bis(methylthio)imidazo[2,1-f][1,2,4]triazine (2.5 g, 17.25 mmol, intermediate 1) was heated to 100° C. for 20 h. The mixture was purified by column chromatography on SiO₂ (10-15% MeOH in CH₂Cl₂) to afford N-(3-methoxy-1-(1-methylpiperidin-4-yl)-1H-pyrazol-4-yl)-7-methyl-2-(methylthio)imidazo[2,1-f][1,2,4]triazin-4-amine as a pale yellow solid (1.5 g, 31%). ¹H NMR (300 MHz, CD₃OD) δ 8.18 (s, 1H), 7.31 (s, 1H), 4.12-4.03 (m, 1H), 3.97 (s, 3H), 3.14-3.06 (m, 2H), 2.57 (s, 3H), 2.48-2.36 (m, 8H), 2.18-2.08 (m, 4H); MS (ESI) m/z 389.38 [M+H]⁺.

Step-4: To a stirred solution of N-(3-methoxy-1-(1-methylpiperidin-4-yl)-1H-pyrazol-4-yl)-7-methyl-2-(methylthio)imidazo[2,1-f][1,2,4]triazin-4-amine (1.4 g, 3.60 mmol) in acetone:water (2:1, 120 mL) was added oxone (2.43 g, 3.96 mmol) at 0° C., and the mixture was stirred at 0° C. for 1 h. Acetone was distilled off under reduced pressure. The reaction was quenched with aq.NaHCO₃ (50 mL) and extracted with ethyl acetate (3×50 mL). The combined organic layer was washed with brine (30 mL), dried over Na₂SO₄ and concentrated under reduced pressure. The resultant residue was purified by column chromatography on SiO₂ (12-16% MeOH in CH₂Cl₂) to afford N-(3-methoxy-1-(1-methylpiperidin-4-yl)-1H-pyrazol-4-yl)-7-methyl-2-(methylsulfinyl)imidazo[2,1-f][1,2,4]triazin-4-amine as an off-white solid (400 mg, 27%). ¹H NMR (300 MHz, CD₃OD) δ 8.30 (s, 1H), 7.48 (s, 1H), 4.20-4.07 (m, 1H), 3.98 (s, 3H), 3.21-3.10 (m, 2H), 3.02 (s, 3H), 2.58-2.42 (m, 8H), 2.23-2.10 (m, 4H); MS (ESI) m/z 405.32 [M+H]⁺.

Step-5: To a stirred solution of N-(3-methoxy-1-(1-methylpiperidin-4-yl)-1H-pyrazol-4-yl)-7-methyl-2-(methylsulfinyl)imidazo[2,1-f][1,2,4]triazin-4-amine (400 mg, 1.79 mmol) in NMP (0.2 mL) was added tert-butyl (3R,4R)-4-fluoropyrrolidin-3-ylcarbamate (303 mg, 2.68 mmol), and the mixture was stirred at 140° C. for 3 h in a sealed tube. To the mixture was added water (25 mL), and the mixture was then extracted with ethyl acetate (2×50 mL). The combined organic layer was washed with brine (30 mL), dried over Na₂SO₄ and concentrated under reduced pressure. The resultant residue was purified by column chromatography on SiO₂ (8-12% MeOH in CH₂Cl₂) to afford tert-butyl ((3R,4R)-4-fluoro-1-(4-((3-methoxy-1-(1-methylpiperidin-4-yl)-1H-pyrazol-4-yl)amino)-7-methylimidazo[2,1-f][1,2,4]triazin-2-yl)pyrrolidin-3-yl)carbamate as a pale brown solid (150 mg, 28%). MS (ESI) m/z 545.44 [M+H]⁺.

Step-6: To a stirred solution of tert-butyl ((3R,4R)-4-fluoro-1-(4-((3-methoxy-1-(1-methylpiperidin-4-yl)-1H-pyrazol-4-yl)amino)-7-methylimidazo[2,1-f][1,2,4]triazin-2-yl)pyrrolidin-3-yl)carbamate (150 mg, 0.275 mmol) in 1,4-dioxane (5 mL) was added 4M HCl in 1,4-dioxane (5 mL) at 0° C., and the mixture was stirred at RT for 1 h. The mixture was concentrated, dissolved in H₂O (15 mL) and washed with EtOAc (15 mL). The aqueous layer was basified with aq.NaHCO₃ solution, extracted with EtOAc (2×30 mL). The combined organic layer was washed with brine (20 mL), dried over sodium sulphate and concentrated under reduced pressure. The resultant residue was triturated with pentane (2 mL) to afford 2-((3R,4R)-3-amino-4-fluoropyrrolidin-1-yl)-N-(3-methoxy-1-(1-methylpiperidin-4-yl)-1H-pyrazol-4-yl)-7-methylimidazo[2,1-f][1,2,4]triazin-4-amine as an off-white solid (70 mg, 57%). MS (ESI) m/z 445.40 [M+H]⁺.

Step-7: To a stirred solution of 2-((3R,4R)-3-amino-4-fluoropyrrolidin-1-yl)-N-(3-methoxy-1-(1-methylpiperidin-4-yl)-1H-pyrazol-4-yl)-7-methylimidazo[2,1-f][1,2,4]triazin-4-amine (0.13 g, 0.321 mmol) in THF (5 mL) was added DIPEA (0.285 mL, 1.605 mmol) followed by a solution of acryloyl chloride (26.06 mg, 0.288 mmol) in THF (1 mL) at 0° C. The mixture was stirred at 0° C. for 5 mins. The mixture was diluted with H₂O (30 mL) and extracted with EtOAc (2×50 mL). The combined organic layer was washed with brine (30 mL), dried over Na₂SO₄ and concentrated under reduced pressure. The resultant residue was purified by triturating with CH₃CN (0.5 mL) to afford N-((3R,4R)-4-fluoro-1-(4-((3-methoxy-1-(1-methylpiperidin-4-yl)-1H-pyrazol-4-yl)amino)-7-methylimidazo[2,1-f][1,2,4]triazin-2-yl)pyrrolidin-3-yl)acrylamide as an off-white solid (56 mg 35%). ¹H NMR (400 MHz, DMSO-d₆) δ 9.06 (br s, 1H), 8.44 (d, J=6.8 Hz, 1H), 7.93 (s, 1H), 7.23 (s, 1H), 6.26-6.10 (m, 2H), 5.63 (dd, J=2.4, 9.2 Hz, 1H), 5.15 (d, J=52 Hz, 1H), 4.50-4.42 (m, 1H), 4.05-3.92 (m, 1H), 3.84 (s, 3H), 3.82-3.55 (m, 4H), 2.89-2.80 (m, 2H), 2.35 (s, 3H), 2.19 (s, 3H), 2.08-1.84 (m, 6H); MS (ESI) m/z 499.05 [M+H]⁺.

### Example 5

### N-((3R,4R)-1-(4-((1-(2-(dimethylamino)ethyl)-3-methoxy-1H-pyrazol-4-yl)amino)-7-methylimidazo[2,1-f][1,2,4]triazin-2-yl)-4-fluoropyrrolidin-3-yl)acrylamide

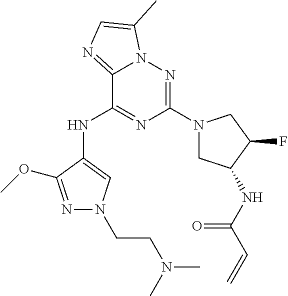 [SMILES: C=CC(=O)N[C@@H]1CN(c2nc(Nc3cn(CCN(C)C)nc3OC)c3ncc(C)n3n2)C[C@H]1F]

Step-1: To a stirred solution of 3-methoxy-4-nitro-1H-pyrazole (10 g, 69.93 mmol) in DMF (100 mL) was added K₂CO₃ (29 g, 210 mmol) followed by 2-chloro-N,N-dimethylethanamine.HCl (12.1 g, 83.9 mmol) portionwise at RT, and the mixture was stirred at 70° C. for 16 h. To this mixture was added H₂O (25 mL), and then the mixture was extracted with EtOAc (2×250 mL). The combined organic layer was washed with brine (100 mL), dried over Na₂SO₄ and concentrated under reduced pressure. The resultant residue was purified by column chromatography on SiO₂ (50% EtOAc in hexane) to afford 2-(3-methoxy-4-nitro-1H-pyrazol-1-yl)-N,N-dimethylethane-1-amine as a pale yellow solid (9.1 g, 61%). ¹H NMR (300 MHz, DMSO-d₆) δ 8.45 (s, 1H), 4.10 (t, J=6.3 Hz, 2H), 3.93 (s, 3H), 2.63 (t, J=6.3 Hz, 2H), 2.16 (s, 6H). MS (ESI) m/z 215.03 [M+H]⁺.

Step-2: To a stirred solution of 2-(3-methoxy-4-nitro-1H-pyrazol-1-yl)-N,N-dimethylethan-1-amine (4 g, 18.69 mmol) in MeOH (120 mL) was added Pd/C (10%, 2.0 g), and the mixture was stirred at RT under H₂ (1 atm) for 3 h. The mixture was filtered through a pad of celite, and the obtained filtrate was evaporated to afford 1-(2-(dimethylamino)ethyl)-3-methoxy-1H-pyrazol-4-amine as pale brown gummy liquid (2.6 g, 75%). MS (ESI) m/z 185.15 [M+H]⁺.

Step-3: A mixture of 1-(2-(dimethylamino)ethyl)-3-methoxy-1H-pyrazol-4-amine (2.6 g, 11.5 mmol) and 7-methyl-2,4-bis(methylthio)imidazo[2,1-f][1,2,4]triazine (2.75 g, 14.95 mmol) was heated to 100° C. for 16 h. The mixture was purified by column chromatography on SiO₂ (5% MeOH in CH₂Cl₂) to afford N-(1-(2-(dimethylamino)ethyl)-3-methoxy-1H-pyrazol-4-yl)-7-methyl-2-(methylthio)imidazo[2,1-f][1,2,4]triazin-4-amine as a pale yellow solid (1.5 g, 36%). ¹H NMR (400 MHz, CD₃OD) δ 8.12 (s, 1H), 7.30 (s, 1H), 4.18 (t, J=6.4 Hz, 2H), 3.98 (s, 3H), 2.91 (t, J=6.4 Hz, 2H), 2.57 (s, 3H), 2.46 (s, 3H), 2.39 (s, 6H); MS (ESI) m/z 363.34 [M+H]⁺.

Step-4: To a stirred solution of N-(1-(2-(dimethylamino)ethyl)-3-methoxy-1H-pyrazol-4-yl)-7-methyl-2-(methylthio)imidazo[2,1-f][1,2,4]triazin-4-amine (1.45 g, 4 mmol) in acetone:water (2:1, 70 mL) was added oxone (2.7 g, 4.4 mmol) at 0° C., and the mixture was stirred at 0° C. for 1 h. Acetone was removed under reduced pressure. The reaction was quenched with aq.NaHCO₃ solution (50 mL), and the obtained solid was filtered. The solid was dissolved in cold 1N NaOH solution and extracted with 10% MeOH in DCM (2×100 mL). The combined organic layer was washed with brine (30 mL), dried over Na₂SO₄ and concentrated under reduced pressure to afford N-(1-(2-(dimethylamino)ethyl)-3-methoxy-1H-pyrazol-4-yl)-7-methyl-2-(methylsulfinyl)imidazo[2,1-f][1,2,4]triazin-4-amine as an off-white solid (500 mg, 28%). ¹H NMR (400 MHz, CD₃OD) δ 8.26 (s, 1H), 7.47 (s, 1H), 4.16 (t, J=6.8 Hz, 2H), 3.99 (s, 3H), 3.03 (s, 3H), 2.80 (t, J=6.8 Hz, 2H), 2.53 (s, 3H), 2.30 (s, 6H); MS (ESI) m/z 379.28 [M+H]⁺.

Step-5: To a stirred solution of N-(1-(2-(dimethylamino)ethyl)-3-methoxy-1H-pyrazol-4-yl)-7-methyl-2-(methylsulfinyl)imidazo[2,1-f][1,2,4]triazin-4-amine (500 mg, 1.32 mmol) in NMP (0.25 mL) was added tert-butyl (3R,4R)-4-fluoropyrrolidin-3-ylcarbamate (408 mg, 1.98 mmol), and the mixture was stirred at 140° C. for 4 h. To the mixture was added H₂O (25 mL), and the mixture was then extracted with EtOAc (2×50 mL). The combined organic layer was washed with brine (30 mL), dried over Na₂SO₄ and concentrated under reduced pressure. The resultant residue was purified by column chromatography on SiO₂ (8-12% MeOH in CH₂Cl₂) to afford tert-butyl ((3R,4R)-1-(4-((1-(2-(dimethylamino)ethyl)-3-methoxy-1H-pyrazol-4-yl)amino)-7-methylimidazo[2,1-f][1,2,4]triazin-2-yl)-4-fluoropyrrolidin-3-yl)carbamate as a pale yellow solid (230 mg, 34%). MS (ESI) m/z 519.41 [M+H]⁺.

Step-6: To a stirred solution of tert-butyl ((3R,4R)-1-(4-((1-(2-(dimethylamino)ethyl)-3-methoxy-1H-pyrazol-4-yl)amino)-7-methylimidazo[2,1-f][1,2,4]triazin-2-yl)-4-fluoropyrrolidin-3-yl)carbamate (230 mg, 0.44 mmol) in 1,4-dioxane (10 mL) was added 4M HCl in 1,4-dioxane (10 mL) at 0° C., and the mixture was stirred at RT for 1 h. The obtained solid was filtered and washed with Et₂O (5 mL). The resulting solid was dissolved in H₂O (25 mL). The mixture was basified with aq.NaHCO₃ solution and extracted with EtOAc (2×30 mL). The combined organic layer was washed with brine (50 mL), dried over sodium sulphate and concentrated under reduced pressure. The resultant residue was triturated with pentane (3 mL) to afford 2-((3R,4R)-3-amino-4-fluoropyrrolidin-1-yl)-N-(1-(2-(dimethylamino)ethyl)-3-methoxy-1H-pyrazol-4-yl)-7-methylimidazo[2,1-f][1,2,4]triazin-4-amine as an off-white solid (95 mg, 51%). ¹H NMR (400 MHz, CD₃OD) δ 8.17 (s, 1H), 7.17 (s, 1H), 5.01 (d, J=52.8 Hz, 1H), 4.15 (t, J=6.4 Hz, 2H), 4.13-3.93 (m, 4H), 3.90-3.78 (m, 2H), 3.72-3.52 (m, 2H), 2.80 (t, J=6.4 Hz, 2H), 2.40 (s, 3H), 2.30 (s, 6H); MS (ESI) m/z 419.31 [M+H]⁺.

Step-7: To a stirred solution of 2-((3R,4R)-3-amino-4-fluoropyrrolidin-1-yl)-N-(1-(2-(dimethylamino)ethyl)-3-methoxy-1H-pyrazol-4-yl)-7-methylimidazo[2,1-f][1,2,4]triazin-4-amine (95 mg, 0.227 mmol) in THF (5 mL) was added DIPEA (0.12 mL, 0.681 mmol) followed by a solution of acryloyl chloride (0.019 g, 0.204 mmol) in THF (1 mL) at 0° C., and the mixture was stirred at 0° C. for 5 mins. The mixture was diluted with H₂O (30 mL) and extracted with EtOAc (2×30 mL). The combined organic layer was washed with brine (30 mL), dried over Na₂SO₄ and concentrated under reduced pressure. The resultant residue was purified by trituration with CH₃CN (0.5 mL) to afford N-((3R,4R)-1-(4-((1-(2-(dimethylamino)ethyl)-3-methoxy-1H-pyrazol-4-yl)amino)-7-methylimidazo[2,1-f][1,2,4]triazin-2-yl)-4-fluoropyrrolidin-3-yl)acrylamide as a white solid (35 mg, 32%). ¹H NMR (400 MHz, DMSO-d₆) δ 9.03 (br s, 1H), 8.46 (d, J=6.8 Hz, 1H), 7.97 (s, 1H), 7.23 (s, 1H), 6.26-6.10 (m, 2H), 5.63 (dd, J=2.4, 9.6 Hz, 1H), 5.13 (d, J=50.4 Hz, 1H), 4.52-4.43 (m, 1H), 4.06 (d, J=6.4 Hz, 2H), 3.84 (s, 3H), 3.83-3.70 (m, 3H), 3.60 (d, J=11.2 Hz, 1H), 2.60 (br s, 2H), 2.35 (s, 3H), 2.17 (s, 6H); MS (ESI) m/z 472.96 [M+H]⁺.

### Example 6

### N-((3R,4R)-1-(9-(bicyclo[1.1.1]pentan-1-yl)-6-((3-methoxy-1-methyl-1H-pyrazol-4-yl)amino)-9H-purin-2-yl)-4-fluoropyrrolidin-3-yl)acrylamide

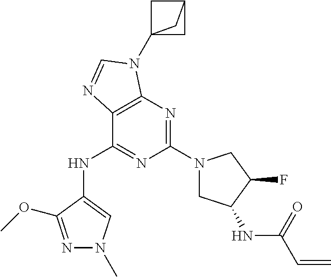 [SMILES: C=CC(=O)N[C@@H]1CN(c2nc(Nc3cn(C)nc3OC)c3ncn(C45CC(C4)C5)c3n2)C[C@H]1F]

Step-1: To a stirred solution of 2,4,6-trichloro-5-nitropyrimidine (2.0 g, 8.810 mmol) in isopropyl alcohol (60 mL) at −78° C. was added a solution of bicyclo[1.1.1]pentan-1-amine hydrochloride salt (1.04 g, 8.810 mmol) in isopropyl alcohol, and the mixture was stirred at −78° C. for 30 mins. The mixture was allowed to warm to RT, then added N,N-diisopropylethylamine (2.27 g, 17.62 mmol) at RT. The mixture was stirred for an additional 30 mins. After completion of reaction, the solvent was removed under reduced pressure and dried to afford N-(bicyclo[1.1.1]pentan-1-yl)-2,6-dichloro-5-nitropyrimidin-4-amine (2.0 g, 7.29 mmol, 84%) as gummy solid. ¹HNMR (300 MHz, CDCl₃) δ 7.96 (S, 1H), 2.60 (S, 1H), 2.25 (S, 6H).

Step-2: To a stirred solution of N-(bicyclo[1.1.1]pentan-1-yl)-2,6-dichloro-5-nitropyrimidin-4-amine (2.0 g, 7.29 mmol) in acetic acid (15 mL) was added iron powder (2.03 g, 36.45 mmol), and the mixture was stirred at RT for 3 h. The mixture was filtered through Celite, and the organic fractions were concentrated. The resulting residue was diluted with ethyl acetate (100 mL), washed with water (50 mL), sat. aq. sodium bicarbonate solution (50 mL) and brine (50 mL), dried over sodium sulphate and concentrated to afford N4-(bicyclo[1.1.1]pentan-1-yl)-2,6-dichloropyrimidine-4,5-diamine as an off-white solid (1.64 g, 6.72 mmol, 92%). ¹H NMR (400 MHz, CDCl₃) δ 5.47 (s, 1H), 3.22 (s, 2H), 2.54 (s, 1H), 2.20 (s, 6H).

Step-3: To a N4-(bicyclo[1.1.1]pentan-1-yl)-2,6-dichloropyrimidine-4,5-diamine (1.6 g, 6.55 mmol) in triethylorthoformate (40 mL) was added 12N aqueous hydrochloric acid (5 mL), and the mixture was stirred at RT for 24 h. The reaction was quenched with ice cold water and extracted with ethylacetate (3×50 mL). The combined organic layers were washed with brine (2×50 mL), dried and concentrated to afford a solid which was further washed with ether (10 mL) and dried to afford 9-(bicyclo[1.1.1]pentan-1-yl)-2,6-dichloro-9H-purine as an off white solid (1.5 g, 5.90 mmol, 91%). ¹H NMR (400 MHz, CDCl₃) δ 8.01 (s, 1H), 2.79 (s, 1H), 2.52 (s, 6H); MS (ESI) m/z 255.03 [M+H]⁺ for ³⁵Cl.

Step-4: To a stirred solution of 9-(bicyclo[1.1.1]pentan-1-yl)-2,6-dichloro-9H-purine (1.6 g, 6.29 mmol) in N-methyl-2-pyrrolidone (40 mL) was added N,N-diisopropylethylamine (1.62 g, 12.58 mmol) and 3-methoxy-1-methyl-1H-pyrazol-4-amine (800 mg, 6.29 mmol) in a microwave vial. The mixture was kept under microwave irradiation at 190° C. for 30 mins. The reaction was quenched with sat. aq. ammonium chloride solution (50 mL) and extracted with ethyl acetate (3×50 mL). The combined organic layers were washed with water (50 mL) and brine (50 mL), dried over sodium sulphate and concentrated. The resultant residue was purified by silica gel chromatography using 0-50% ethyl acetate in hexane as the eluent to afford 9-(bicyclo[1.1.1]pentan-1-yl)-2-chloro-N-(3-methoxy-1-methyl-1H-pyrazol-4-yl)-9H-purin-6-amine (1.8 g, 5.21 mmol, 83%) as an off white solid. ¹H NMR (400 MHz, CDCl₃) δ 8.0 (s, 1H), 7.69 (s, 1H), 7.25 (s, 1H), 3.97 (s, 3H), 3.78 (s, 3H), 2.72 (s, 1H), 2.46 (s, 6H); MS (ESI) m/z 346.2 [M+H]⁺ for ³⁵Cl.

Step-5: To a stirred solution of 9-(bicyclo[1.1.1]pentan-1-yl)-2-chloro-N-(3-methoxy-1-methyl-1H-pyrazol-4-yl)-9H-purin-6-amine (1.8 g, 5.21 mmol) in N-methyl-2-pyrrolidone (30 mL) was added tert-butyl 4-fluoropyrrolidin-3-ylcarbamate (trans, racemic, 1.06 g 5.21 mmol) in a sealed tube at 140° C. for 3 h. The reaction was quenched with sat. aq. ammonium chloride solution (100 mL) and extracted with ethyl acetate (2×100 mL). The combined organic layers were washed with water (100 mL), brine (100 mL), dried over sodium sulphate and concentrated to afford tert-butyl (1-(9-(bicyclo[1.1.1]pentan-1-yl)-6-((3-methoxy-1-methyl-1H-pyrazol-4-yl)amino)-9H-purin-2-yl)-4-fluoropyrrolidin-3-yl)carbamate (2.0 g (crude), 3.89 mmol, 75%) as an off-white solid. MS (ESI) m/z 514.07 [M+H]⁺.

Step-6: To a stirred solution of tert-butyl (1-(9-(bicyclo[1.1.1]pentan-1-yl)-6-((3-methoxy-1-methyl-1H-pyrazol-4-yl)amino)-9H-purin-2-yl)-4-fluoropyrrolidin-3-yl)carbamate (2.0 g, 3.89 mmol) in 1,4-dioxane (20 mL) was added 4M HCl in 1,4-dioxane (15 mL), and the mixture was stirred at RT for 2 h. The mixture was concentrated and triturated with diethyl ether (10 mL) to afford 2-(3-amino-4-fluoropyrrolidin-1-yl)-9-(bicyclo[1.1.1]pentan-1-yl)-N-(3-methoxy-1-methyl-1H-pyrazol-4-yl)-9H-purin-6-amine hydrochloride. The resulting residue was diluted with water, basified with sat. aq. sodium bicarbonate solution (10 mL) and extracted with ethyl acetate (2×100 mL). The combined organic layers were washed with water (50 mL) and brine (50 mL). The organic layer was dried over sodium sulphate and concentrated to afford 2-(3-amino-4-fluoropyrrolidin-1-yl)-9-(bicyclo[1.1.1]pentan-1-yl)-N-(3-methoxy-1-methyl-1H-pyrazol-4-yl)-9H-purin-6-amine (800 mg, 1.93 mmol, 50%) as an off white solid. ¹H NMR (300 MHz, DMSO-d₆) δ 7.94 (s, 1H), 7.82 (s, 1H), 7.75 (s, 1H), 5.05-4.79 (m, 1H), 3.84-3.76 (m, 5H), 3.75-3.67 (m, 4H), 3.66-3.56 (s, 3H), 3.42 (br d, J=11.4 Hz, 1H), 2.69 (s, 1H), 2.38 (s, 6H); MS (ESI) m/z 414.34 [M+H]⁺.

Step-7: To a stirred solution of 2-(3-amino-4-fluoropyrrolidin-1-yl)-9-(bicyclo[1.1.1]pentan-1-yl)-N-(3-methoxy-1-methyl-1H-pyrazol-4-yl)-9H-purin-6-amine (800 mg, 1.93 mmol) in mixture of tetrahydrofuran:water (1:1 30 mL) at 0° C. was added a solution of acryloyl chloride (1.5 mL, 1.544 mmol) in tetrahydrofuran (1.5 mL). The mixture was stirred at 0° C. for 1 h. The mixture was then diluted with water (30 mL) and extracted with ethyl acetate (3×30 mL). The combined organic layers were washed with brine (30 mL), dried over sodium sulphate and concentrated under reduced pressure. The obtained residue was purified by Reveleris C-18 reversed phase column using 55% acetonitrile in aqueous formic acid (0.1%) to afford racemic N-(1-(9-(bicyclo[1.1.1]pentan-1-yl)-6-((3-methoxy-1-methyl-1H-pyrazol-4-yl)amino)-9H-purin-2-yl)-4-fluoropyrrolidin-3-yl)acrylamide (310 mg, 0.663 mmol, 34%) as an off white solid. ¹H NMR (300 MHz, CDCl₃) δ 7.82 (s, 1H), 7.41 (s, 1H), 7.12 (s, 1H), 6.36 (d, J=16.8 Hz, 1H), 6.15-6.06 (m, 2H), 5.69 (d, J=10.2 Hz, 1H), 5.23 (d, J=51.6 Hz, 1H), 4.73 (s, 1H), 3.96 (s, 3H), 3.95-3.82 (m, 4H), 3.75 (s, 3H), 2.67 (s, 1H), 2.41 (s, 6H); MS (ESI) m/z 468.31 [M+H]⁺. The above racemic compound was purified by chiral SFC (Chiralpak-AD-H(250×4.6)mm:5 micron, 100% ethanol) to afford N-((3R,4R)-1-(9-(bicyclo[1.1.1]pentan-1-yl)-6-((3-methoxy-1-methyl-1H-pyrazol-4-yl)amino)-9H-purin-2-yl)-4-fluoropyrrolidin-3-yl)acrylamide (92 mg) as an off-white solid. MS(ESI) m/z 468.31[M+H]⁺.

### Example 7

### N-((3R,4R)-1-(7-ethyl-4-((3-methoxy-1-((R)-1-methylpyrrolidin-3-yl)-1H-pyrazol-4-yl)amino)imidazo[2,1-f][1,2,4]triazin-2-yl)-4-fluoropyrrolidin-3-yl)acrylamide

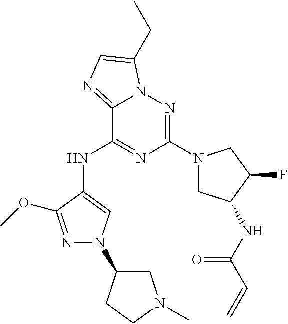 [SMILES: C=CC(=O)N[C@@H]1CN(c2nc(Nc3cn([C@@H]4CCN(C)C4)nc3OC)c3ncc(CC)n3n2)C[C@H]1F]

Step-1: To a solution of butyraldehyde (100 g, 1.39 mol) and MS 4 Å (44 g) in MeOH (800 mL), was added Br₂ (72 mL, 1.39 mol) at 75° C., and the mixture was stirred for 5 h followed by stirring at RT for 16 h. To the mixture was added K₂CO₃ (96 g, 694.4 mmol) and it stirred at RT for 3 h. The mixture was filtered, and the filtrate diluted with brine solution (500 mL) and extracted with pentanes (3×300 mL). The combined organic layer was dried over Na₂SO₄ and concentrated under reduced pressure. The resulting residue underwent fractional distillation to afford 2-bromo-1,1-dimethoxybutane as a colorless liquid (40 g, 15%). ¹H NMR (300 MHz, CDCl₃) δ 4.39 (d, J=5.7 Hz, 1H), 3.95-3.89 (m, 1H), 3.43 (s, 6H), 2.05-1.96 (m, 1H), 1.81-1.71 (m, 1H) 1.06 (t, J=7.5 Hz, 3H).

Step-2: To a stirred solution of 3,5-bis(methylthio)-1,2,4-triazin-6-amine (5.1 g, 27.12 mmol, see step 4 in the synthesis intermediate 1) and 2-bromo-1,1-dimethoxybutane (16.0 g, 81.38 mmol,), in CH₃CN (50 mL) was added (+/−)-camphor-10-sulfonic acid (0.630 g, 2.71 mmol) and H₂O (48 mL, 2.71 mmol). The mixture was heated at 85° C. for 16 h. The mixture was cooled to RT and concentrated under reduced pressure to obtain a crude residue. The residue was diluted with EtOAc and washed with H₂O (2×15 mL). The organic layer was dried over Na₂SO₄, concentrated under reduced pressure, and the resulting residue was purified by column chromatography on SiO₂ (40% EtOAc in hexane) to afford 7-ethyl-2,4-bis(methylthio)imidazo[2,1-f][1,2,4]triazine as a pale brown solid (2.9 g, 44%). ¹H NMR (300 MHz, CDCl₃) δ 7.42 (s, 1H), 2.94 (q, J=7.8 Hz, 2H), 2.66 (s, 3H), 2.60 (s, 3H), 1.36 (t, J=7.8 Hz, 3H), MS (ESI) m/z 241.52 [M+H]⁺.

Step-3: A mixture of 7-ethyl-2,4-bis(methylthio)imidazo[2,1-f][1,2,4]triazine (2.9 g, 12.08 mmol) and (R)-3-methoxy-1-(1-methylpyrrolidin-3-yl)-1H-pyrazol-4-amine (5.32 g, 27.0 mmol, synthesized by following the procedure as described in Example-3) was heated at 100° C. for 20 h. The mixture was purified by column chromatography on SiO₂ (10-15% MeOH in DCM) to afford (R)-7-ethyl-N-(3-methoxy-1-(1-methylpyrrolidin-3-yl)-1H-pyrazol-4-yl)-2-(methylthio)imidazo[2,1-f][1,2,4]triazin-4-amine as a pale yellow solid (1.25 g, 27%). ¹H NMR (300 MHz, DMSO-d₆) δ 9.78 (s, 1H), 7.91 (s, 1H), 7.39 (s, 1H), 4.95-4.80 (m, 1H), 4.08 (q, J=5.1 Hz 1H), 3.83 (s, 3H), 3.16 (d, J=5.1 Hz, 2H), 3.20-2.95 (m, 2H), 2.84 (q, J=7.2 Hz, 2H), 2.49 (s, 3H), 2.48 (s, 3H), 2.20-2.11 (m, 1H), 1.28 (t, J=7.2 Hz, 3H); MS (ESI) m/z 389.45 [M+H]⁺.

Step-4: To a stirred solution of (R)-7-ethyl-N-(3-methoxy-1-(1-methylpyrrolidin-3-yl)-1H-pyrazol-4-yl)-2-(methylthio)imidazo[1,2-f][1,2,4]triazin-4-amine (1.25 g, 3.22 mmol) in acetone:water (2:1, 50 mL) was added oxone (1.58 g, 2.58 mmol), and the mixture stirred at 0° C. for 1 h. Acetone was removed under reduced pressure. The reaction was quenched with aq.NaHCO₃ solution (15 mL) and extracted with EtOAc (2×50 mL). The combined organic layer was washed with brine (30 mL), dried over Na₂SO₄ and concentrated under reduced pressure to afford 7-ethyl-N-(3-methoxy-1-((R)-1-methylpyrrolidin-3-yl)-1H-pyrazol-4-yl)-2-(methylsulfinyl)imidazo[2,1-f][1,2,4]triazin-4-amine as an off-white solid (700 mg, 54% crude). ¹H NMR (300 MHz, CDCl₃) δ 8.29 (d, J=3.0 Hz, 1H), 8.10 (br s, 1H), 7.41 (s, 1H), 4.74-4.69 (m, 1H), 3.98 (s, 3H), 3.03-2.95 (m, 2H), 3.00 (s, 3H), 2.91-2.78 (m, 3H), 2.62-2.52 (m, 1H), 2.50-2.22 (m, 1H), 2.40 (s, 3H), 2.12-2.02 (m, 1H), 1.38 (t, J=7.5 Hz, 3H); MS (ESI) m/z 405.61 [M+H]⁺.

Step-5: To a stirred solution of 7-ethyl-N-(3-methoxy-1-((R)-1-methylpyrrolidin-3-yl)-1H-pyrazol-4-yl)-2-(methylsulfinyl)imidazo[2,1-f][1,2,4]triazin-4-amine (450 mg, 1.113 mmol) in NMP (1 mL) was added tert-butyl ((3R,4R)-4-fluoropyrrolidin-3-yl)carbamate (568 mg, 2.784 mmol), and the mixture was stirred at 140° C. for 4 h in a sealed tube. To the mixture was added H₂O (15 mL). The mixture was extracted with EtOAc (2×50 mL). The combined organic layer was washed with brine (20 mL), dried over Na₂SO₄ and concentrated under reduced pressure. The resultant residue was purified by column chromatography on SiO₂ (5-10% MeOH in DCM) to afford tert-butyl ((3R,4R)-1-(7-ethyl-4-((3-methoxy-1-((R)-1-methylpyrrolidin-3-yl)-1H-pyrazol-4-yl)amino)imidazo[2,1-f][1,2,4]triazin-2-yl)-4-fluoropyrrolidin-3-yl)carbamate as a pale brown solid (250 mg, 41%). MS (ESI) m/z 545.58 [M+H]⁺.

Step-6: To a stirred solution of tert-butyl ((3R,4R)-1-(7-ethyl-4-((3-methoxy-1-((R)-1-methylpyrrolidin-3-yl)-1H-pyrazol-4-yl)amino)imidazo[2,1-f][1,2,4]triazin-2-yl)-4-fluoropyrrolidin-3-yl)carbamate (0.520 g, 0.955 mmol) in 1,4-dioxane (5 mL) was added 4M HCl in 1,4-dioxane (5 mL) at 0° C., and the mixture was stirred at RT for 1 h. The mixture was concentrated, dissolved in H₂O (2.5 mL) and washed with EtOAc (15 mL). The aqueous layer was basified with aq.NaHCO₃ solution and extracted with EtOAc (2×30 mL). The combined organic layer was washed with brine (50 mL), dried over Na₂SO₄ and concentrated under reduced pressure. The resultant residue was purified by column chromatography on SiO₂ (3-5% MeOH in DCM) to afford 2-((3R,4R)-3-amino-4-fluoropyrrolidin-1-yl)-7-ethyl-N-(3-methoxy-1-((R)-1-methylpyrrolidin-3-yl)-1H-pyrazol-4-yl)imidazo[2,1-f][1,2,4]triazin-4-amine as an off-white solid (250 mg, 59%). MS (ESI) m/z 445.14 [M+H]⁺.

Step-7: To a stirred solution of 2-((3R,4R)-3-amino-4-fluoropyrrolidin-1-yl)-7-ethyl-N-(3-methoxy-1-((R)-1-methylpyrrolidin-3-yl)-1H-pyrazol-4-yl)imidazo[2,1-f][1,2,4]triazin-4-amine (0.250 g, 0.563 mmol) in THF (5 mL) was added DIPEA (0.58 mL, 3.378 mmol) followed by a solution of acryloyl chloride (0.0352 mL, 0.422 mmol) at 0° C. The mixture stirred at 0° C. for 5 mins. H₂O (10 mL) was added, and the mixture was extracted with EtOAc (2×50 mL). The combined organic layer was washed with brine (20 mL), dried over Na₂SO₄ and concentrated under reduced pressure. The resultant residue was purified by column chromatography on SiO₂ (5-10% MeOH in DCM) and triturating with EtOAc (0.1 mL) in pentane (5.0 mL) to afford N-((3R,4R)-1-(7-ethyl-4-((3-methoxy-1-((R)-1-methylpyrrolidin-3-yl)-1H-pyrazol-4-yl)amino)imidazo[2,1-f][1,2,4]triazin-2-yl)-4-fluoropyrrolidin-3-yl)acrylamide as a green solid (30 mg, 11%). ¹H NMR (300 MHz, DMSO-d₆) δ 9.10 (s, 1H), 8.47 (d, J=6.6 Hz, 1H), 8.12 (s, 1H), 7.25 (s, 1H), 6.27-6.10 (m, 2H), 5.63 (dd, J=9.0, 2.4 Hz, 1H), 5.15 (d, J=51.9 Hz, 1H), 4.62-4.39 (m, 1H), 4.55-4.40 (m, 1H), 3.86 (s, 3H), 3.80-3.55 (m, 4H), 2.82-2.65 (m, 5H), 2.60-2.20 (m, 5H), 2.10-1.90 (m, 1H), 1.40-1.28 (t, 3H); MS (ESI) m/z 499.40 [M+H]⁺.

General methods and conditions for preparing compounds of Formula (I), or a pharmaceutically acceptable salt thereof, are shown herein in Schemes A-F. Compounds that can be prepared using one of more of methods shown in Schemes A-F include the following:

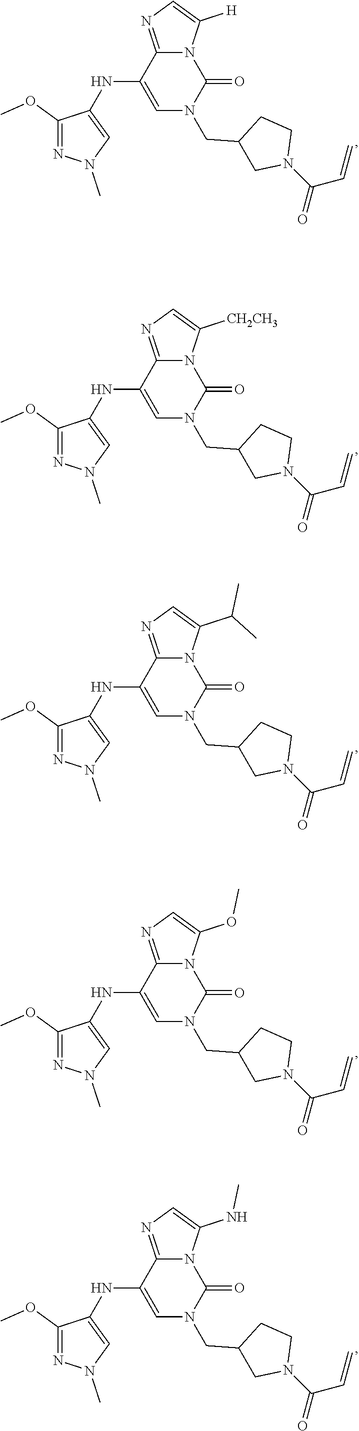

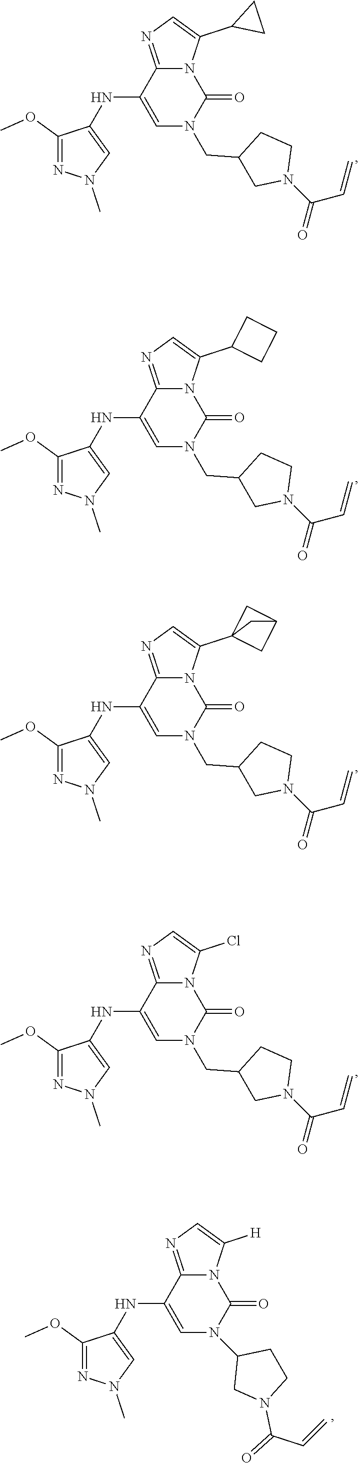

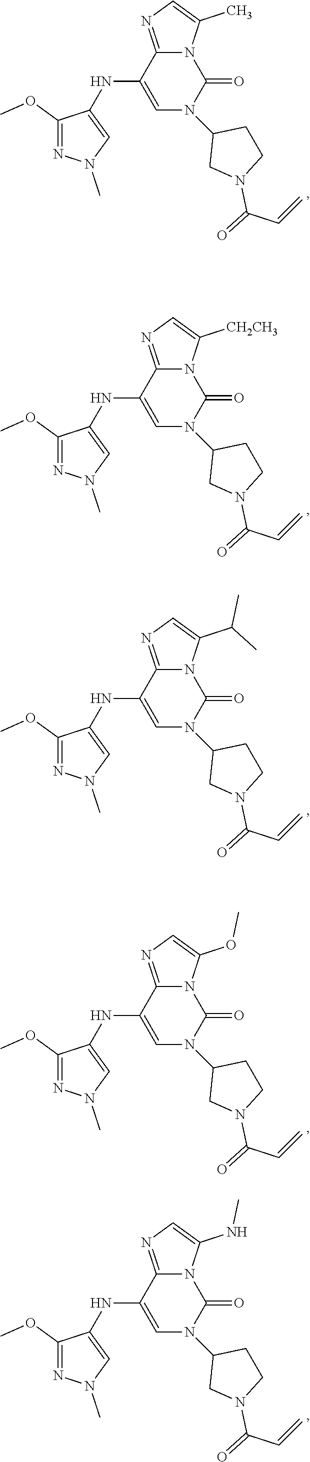

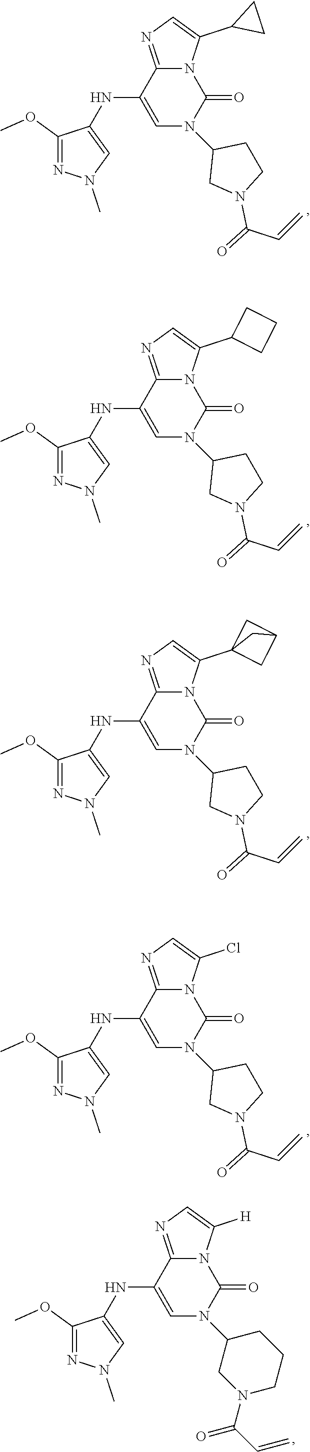

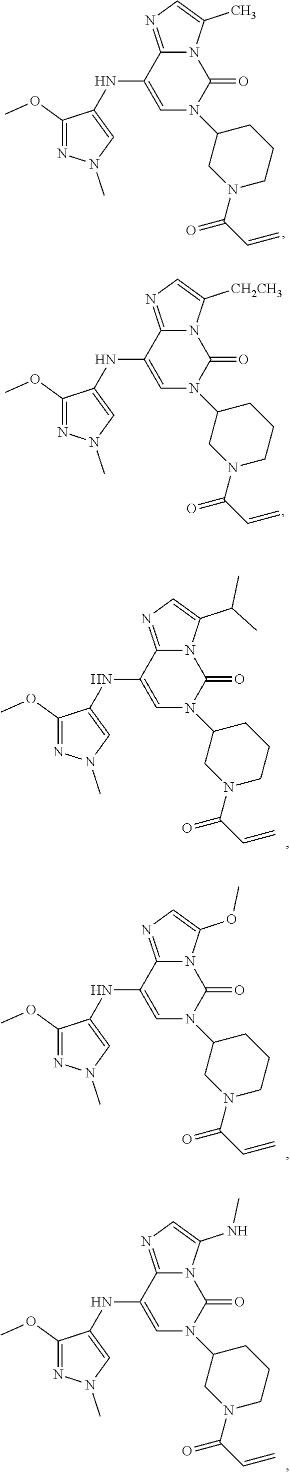

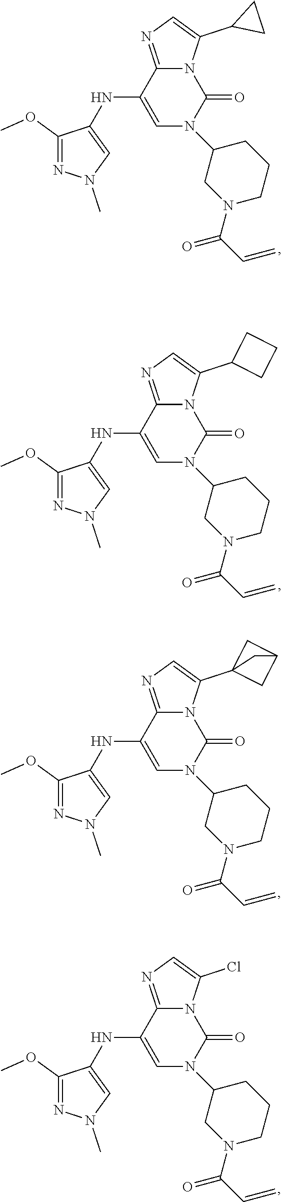

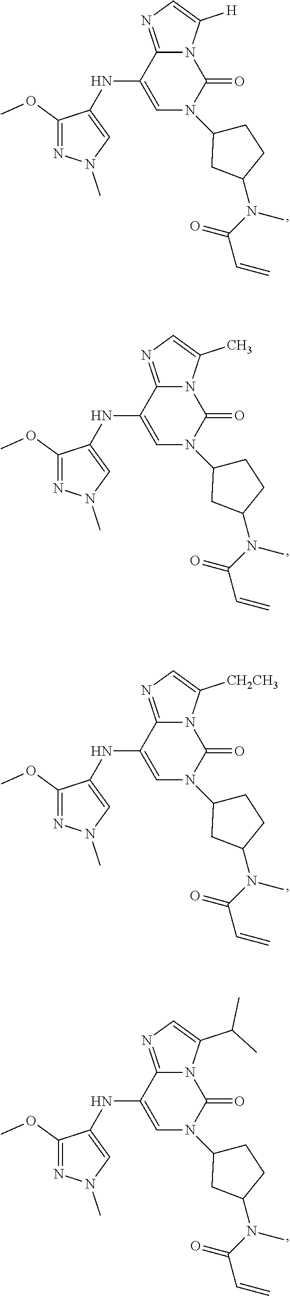

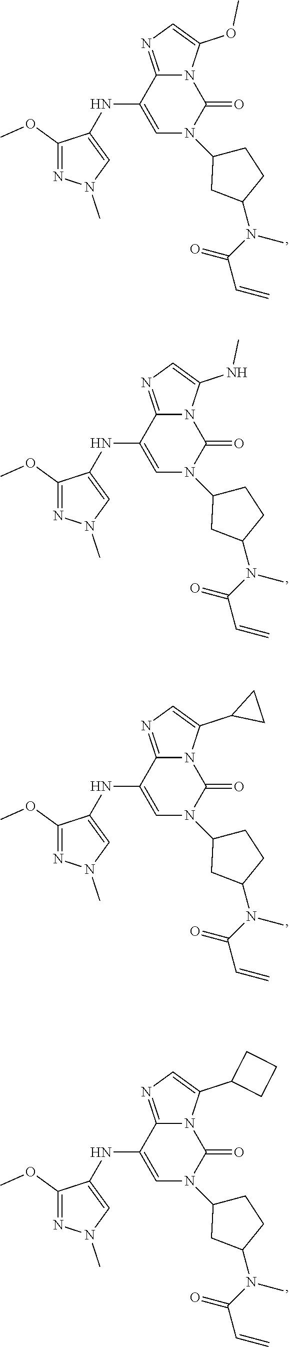

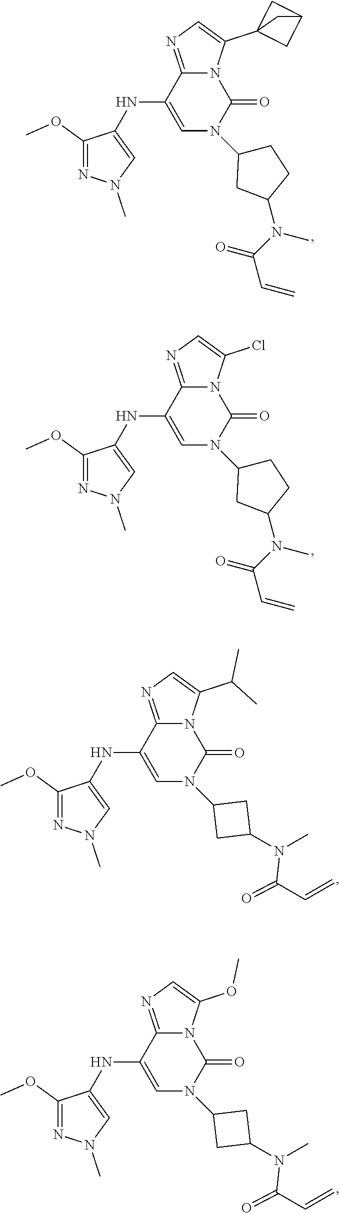

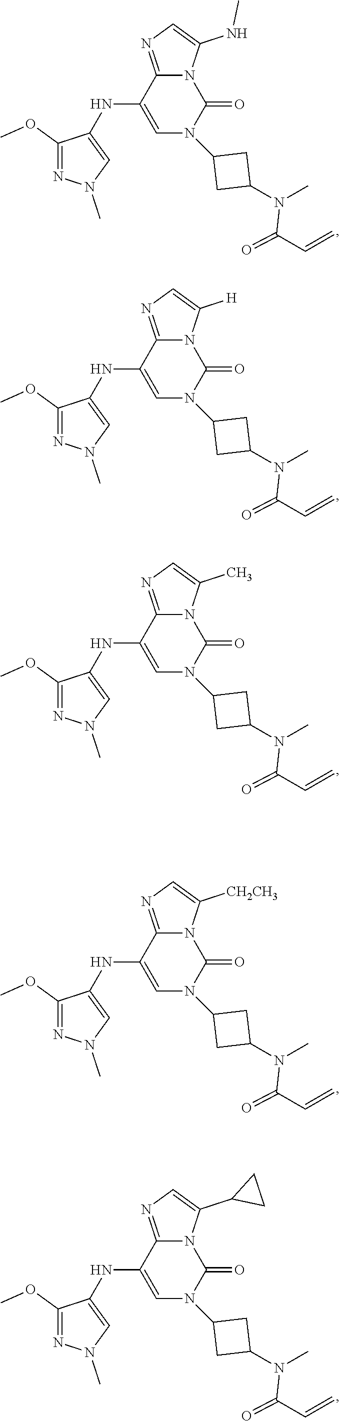

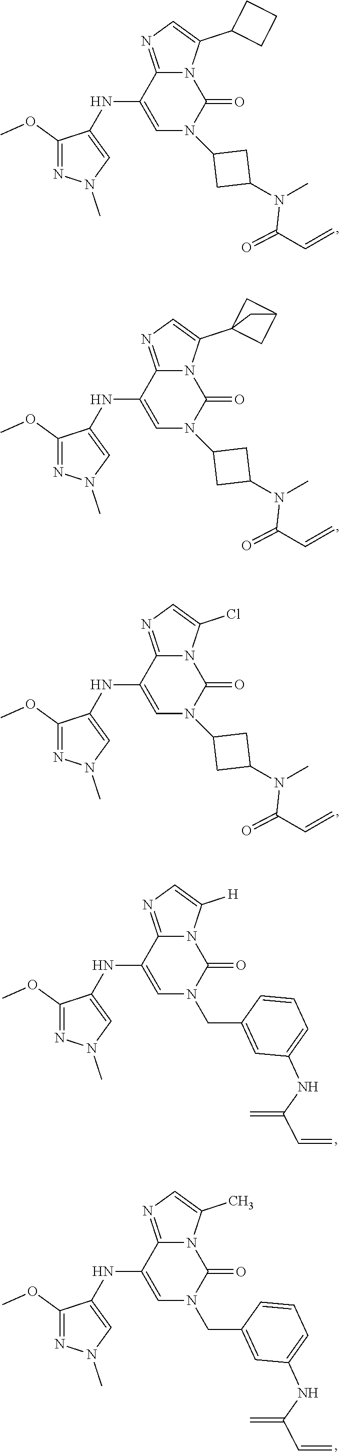

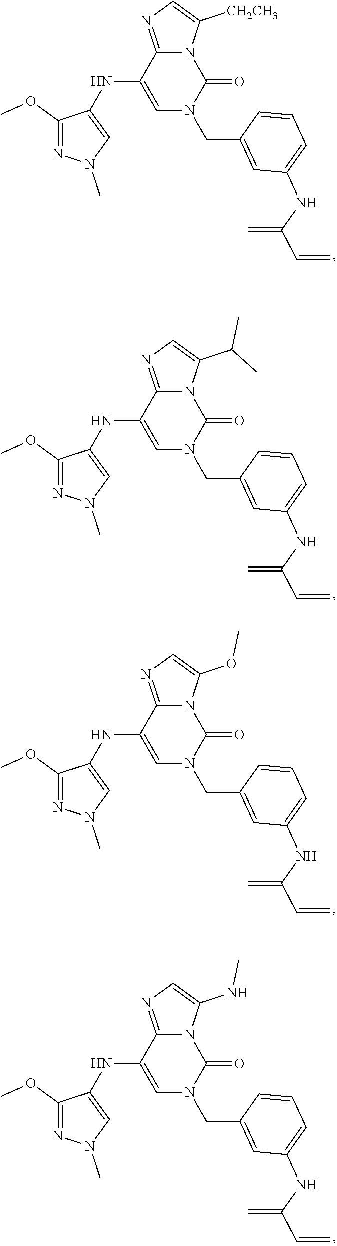

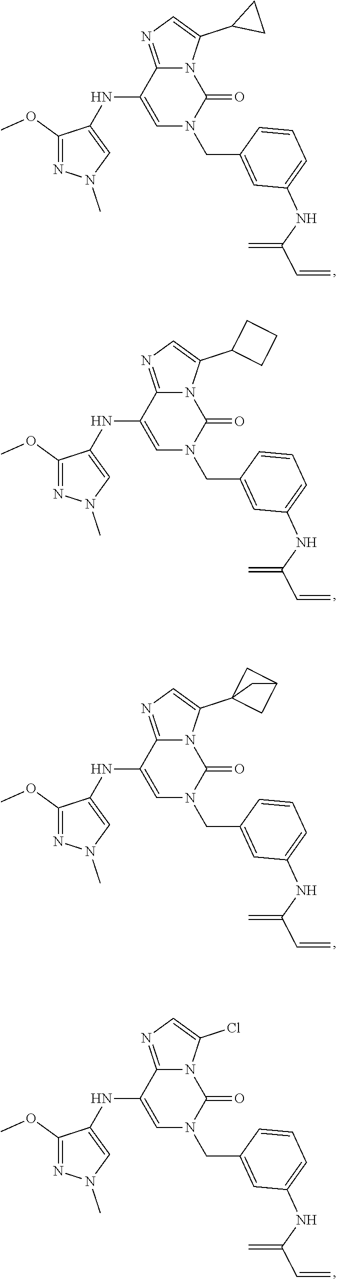

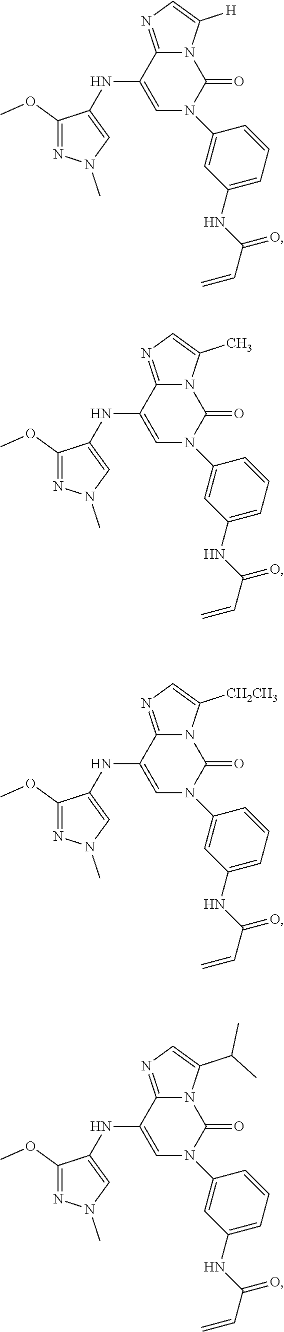 [SMILES: C=CC(=O)Nc1cccc(-n2cc(Nc3cn(C)nc3OC)c3nccn3c2=O)c1 ; C=CC(=O)Nc1cccc(-n2cc(Nc3cn(C)nc3OC)c3ncc(C)n3c2=O)c1 ; C=CC(=O)Nc1cccc(-n2cc(Nc3cn(C)nc3OC)c3ncc(CC)n3c2=O)c1 ; C=CC(=O)Nc1cccc(-n2cc(Nc3cn(C)nc3OC)c3ncc(C(C)C)n3c2=O)c1]

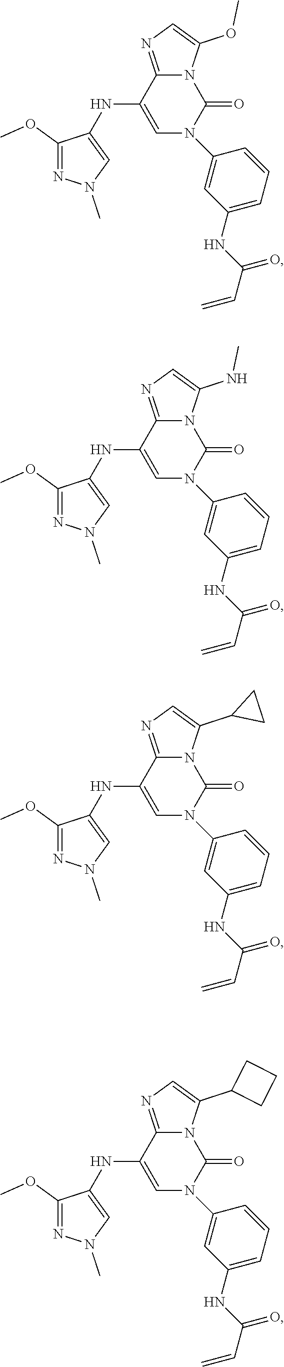

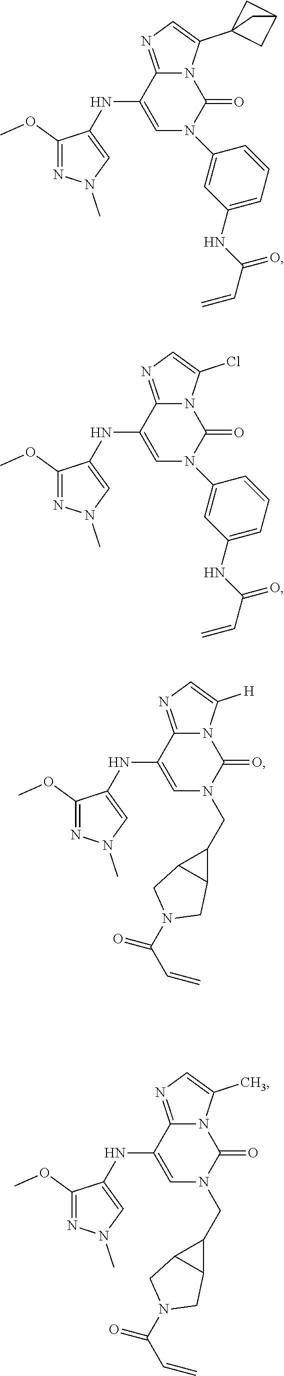

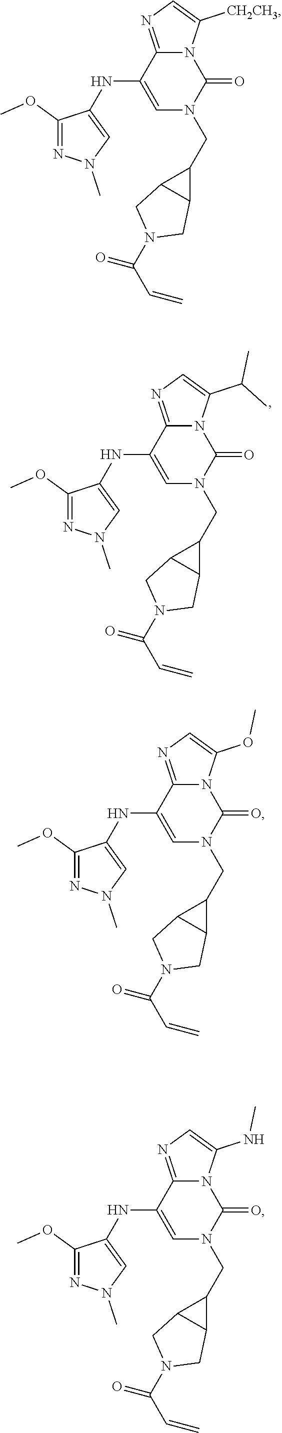

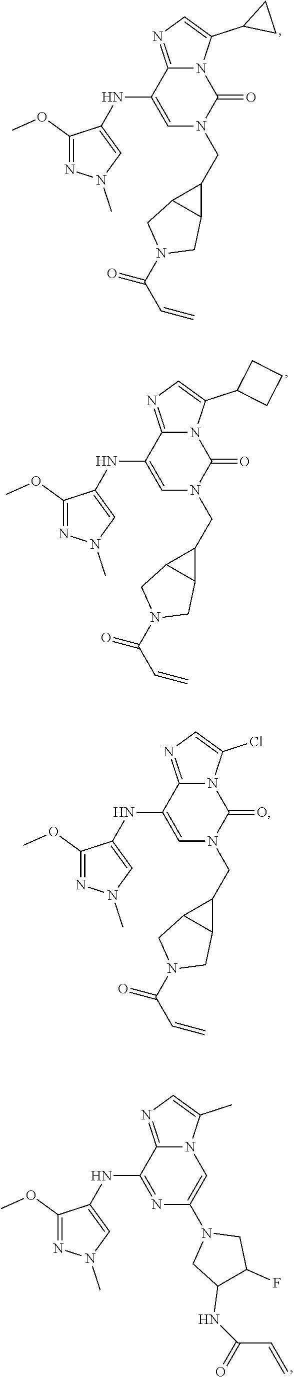 [SMILES: C=CC(=O)N1CC2C(C1)C2Cn1cc(Nc2cn(C)nc2OC)c2ncc(C3CC3)n2c1=O ; C=CC(=O)N1CC2C(C1)C2Cn1cc(Nc2cn(C)nc2OC)c2ncc(C3CCC3)n2c1=O ; C=CC(=O)N1CC2C(C1)C2Cn1cc(Nc2cn(C)nc2OC)c2ncc(Cl)n2c1=O ; C=CC(=O)NC1CN(c2cn3c(C)cnc3c(Nc3cn(C)nc3OC)n2)CC1F]

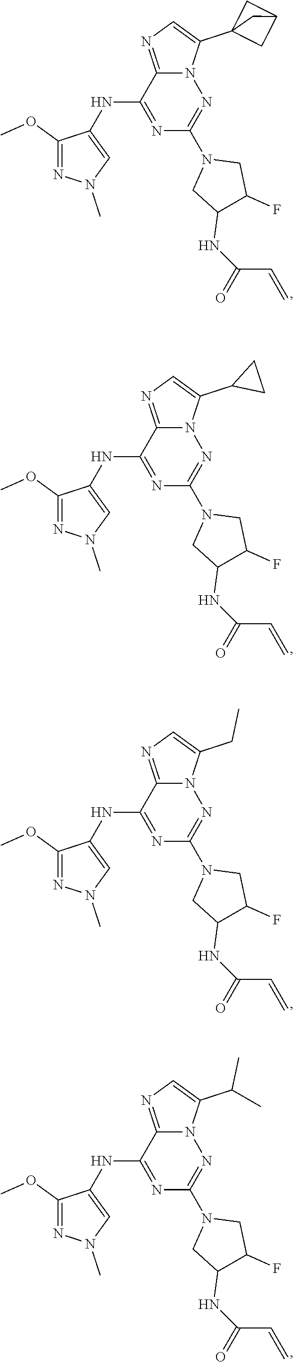

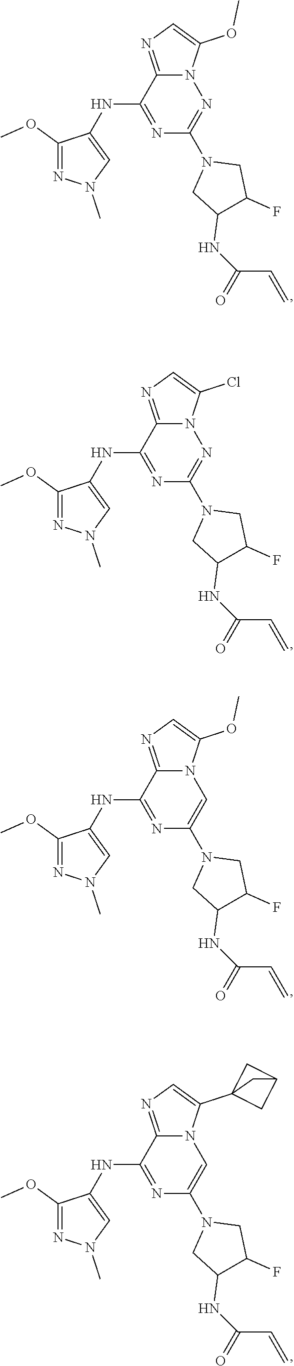

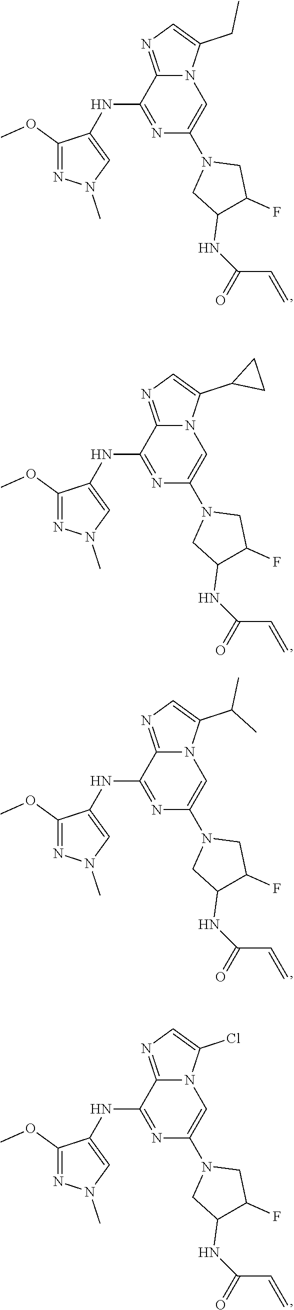

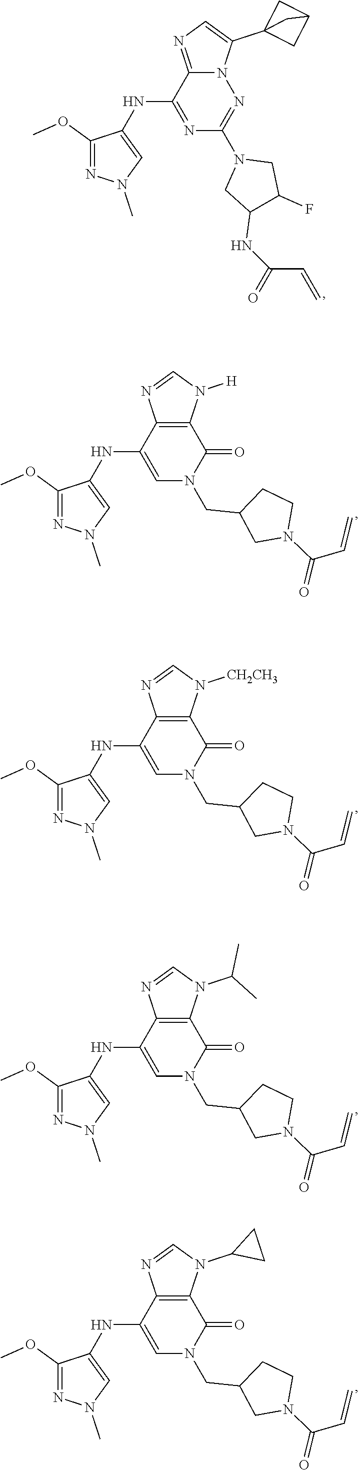

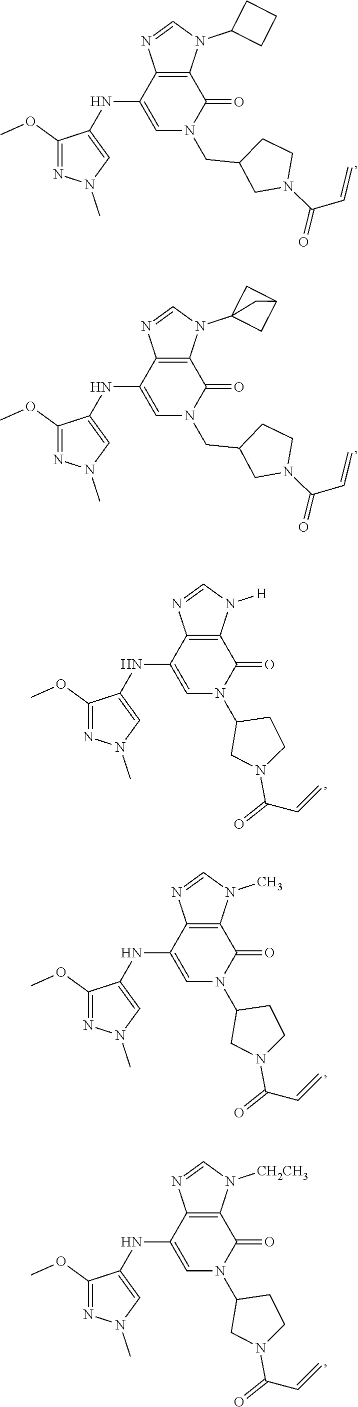

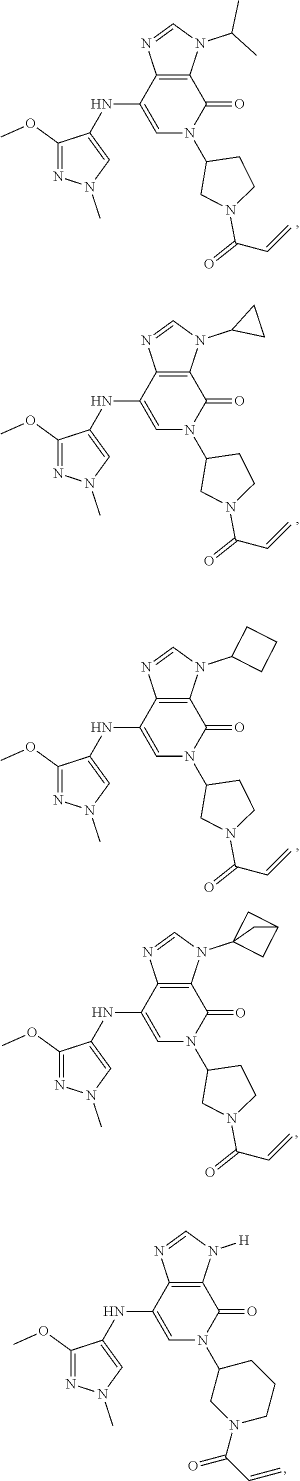

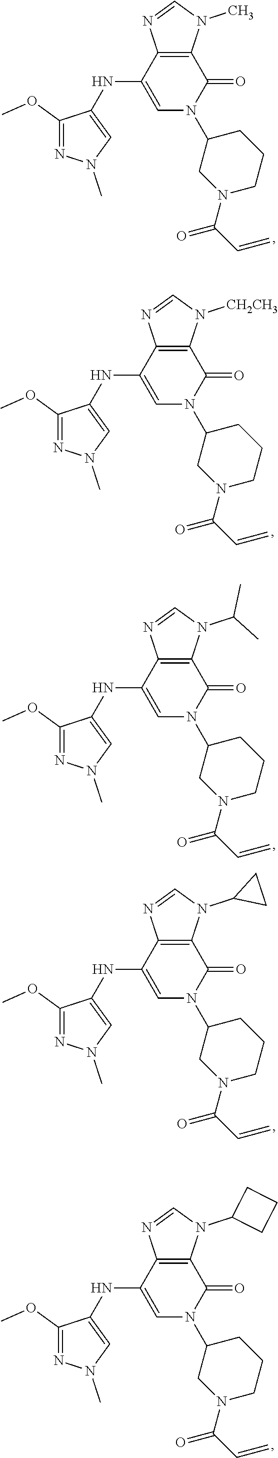

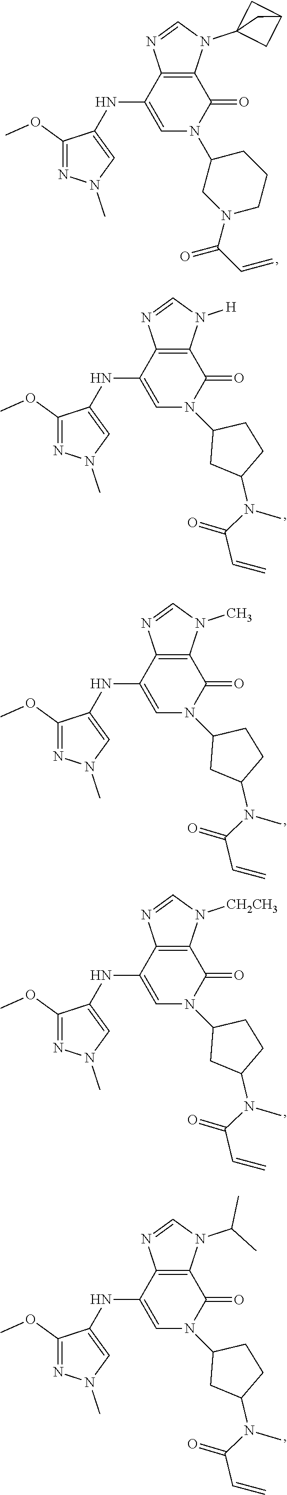

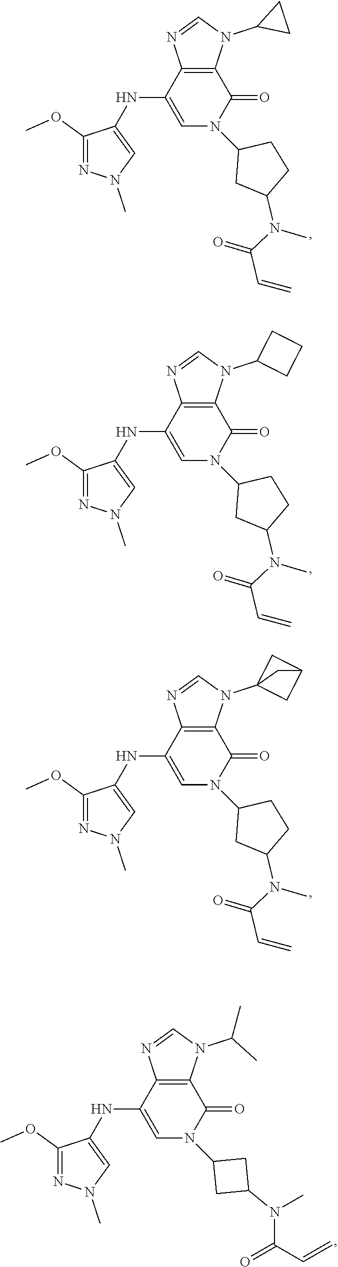

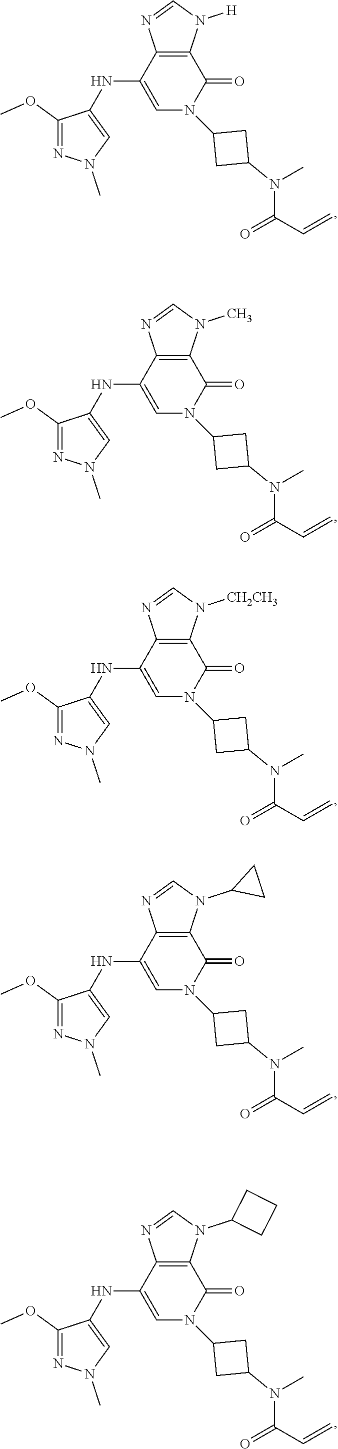

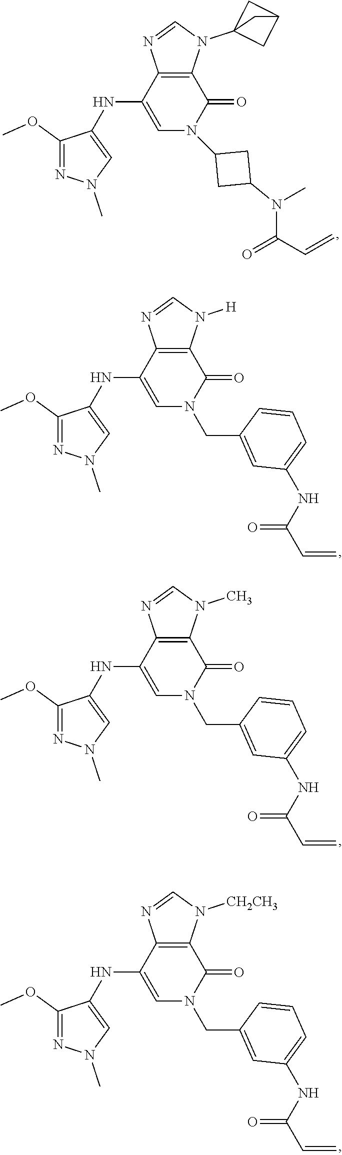

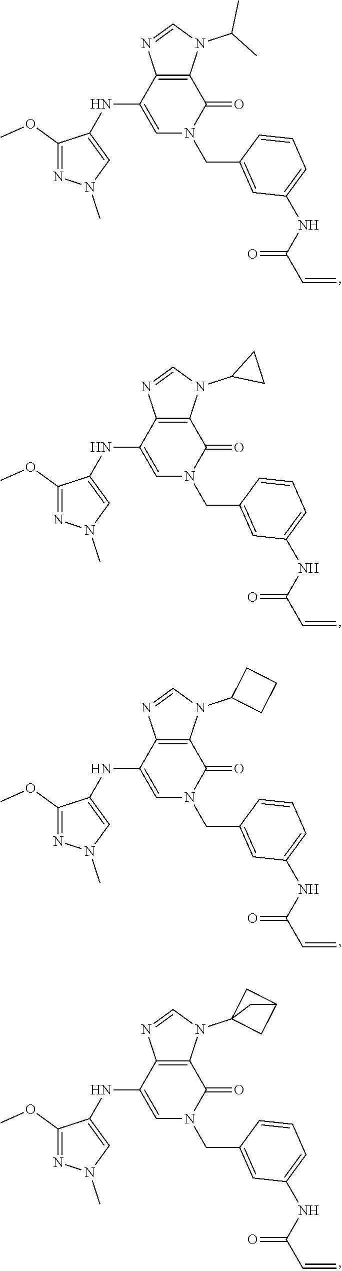

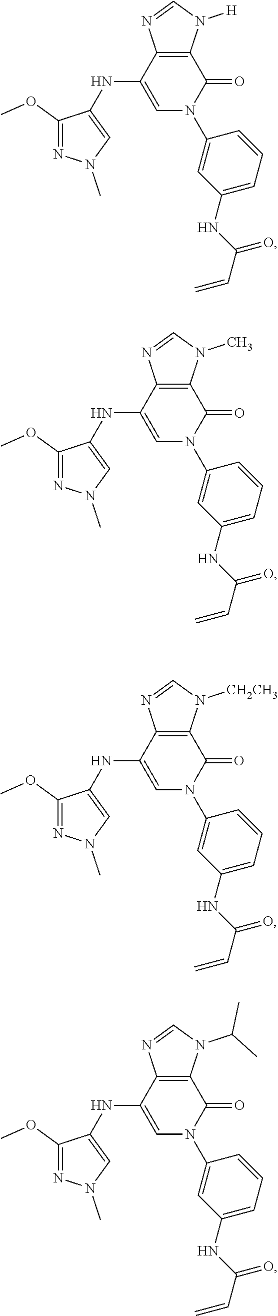

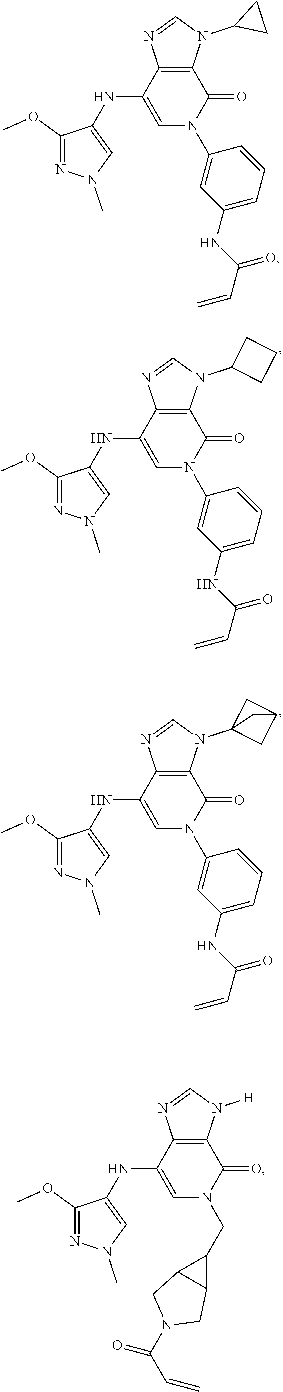

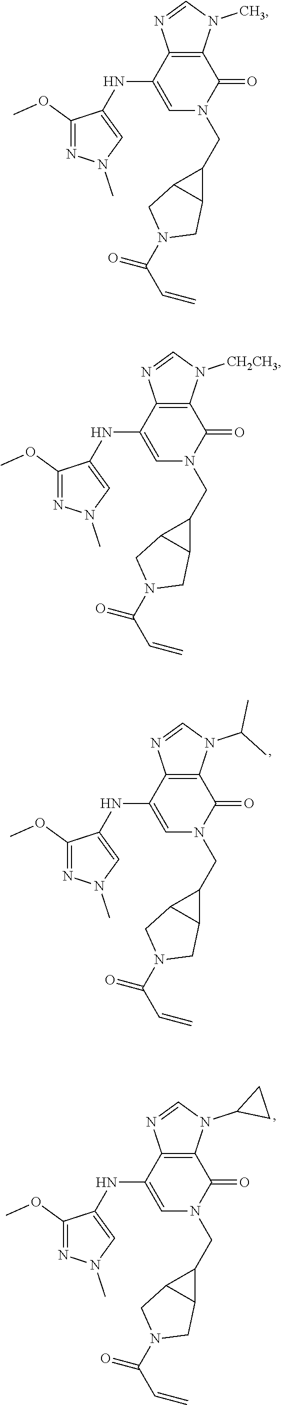

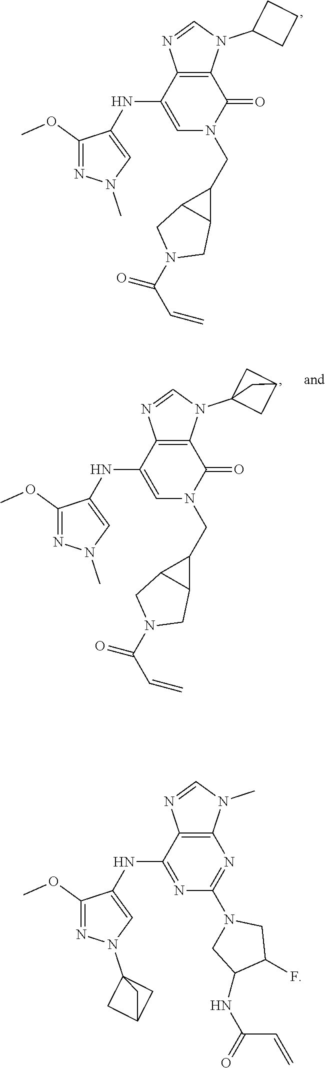 [SMILES: C=CC(=O)N1CC2C(C1)C2Cn1cc(Nc2cn(C)nc2OC)c2ncn(C3CCC3)c2c1=O ; C=CC(=O)N1CC2C(C1)C2Cn1cc(Nc2cn(C)nc2OC)c2ncn(C34CC(C3)C4)c2c1=O ; C=CC(=O)NC1CN(c2nc(Nc3cn(C45CC(C4)C5)nc3OC)c3ncn(C)c3n2)CC1F]

### Example A

### EGFR Biochemical Enzyme Assay Protocol

The inhibitory activity of a compound against EGFR (T790M/L858R) were determined with CisBio HTRF (homogenous time-resolved fluorescence) KinEASE TK (#62TKOPEC). The enzyme reaction contained recombinant N-terminal GST-tagged human EGFR (T790M/L858R), which phosphorylates the HTRF tyrosine kinase biotinylated substrate.

The sequence of the substrate is proprietary to CisBio. Test compounds were serially diluted in 100% (v/v) DMSO before being acoustically dispensed from an Echo 555 (Labcyte) into black Corning 1536-well assay plates. Kinase activity assays were performed in a total reaction volume of 3 μL per well. A 1.5 μL enzyme reaction consisted of 1.6 nM EGFR (T970M, L858R), 1 mM DTT, and 10 mM MgCl₂. A 1.5 μL substrate mix consisted of 1 μM TK substrate, 30 μM ATP, 1 mM DTT, and 10 mM MgCl₂. Following a 50 mins incubation, 3 μL of stop mix was added, which consisted of 250 nM Strep-XL665 and TK Ab-Cryptate diluted in kit detection buffer. The plates were incubated for 1 h before being read on Pherastar using standard HTRF settings. N-terminal GST-tagged recombinant human EGF receptor, with amino acids 696-end containing the T790M and L858R mutations, was obtained from Millipore.

Compounds of Formula (I) are active in this assay as provided in Table 1, where A=IC₅₀≤10 nM; B=IC₅₀>10 nM and <100 nM; and C=IC₅₀≥100 nM.

TABLE 1

| | T790M/L | | | | | |
|---|---|---|---|---|---|---|
| | 858R | L858R | Del-19 | Wt | IGF1R | INSR |
| Example # | (nM) | (nM) | (nM) | (nM) | (nM) | (nM) |
| 1 | A | A | A | A | C | C |
| 2 | A | — | A | A | C | C |
| 3 | A | — | A | A | C | C |
| 4 | A | — | A | A | C | C |
| 5 | A | — | A | A | C | C |
| 6 | A | A | A | A | C | C |

### Example B

### p-EGFR: Target Engagement Assay (Cell-Based Phospho-EGFR Assay) Western Blot

Cell lines used as follows: A431 (WT), H1975 (L858R/T790M), PC9 (E746-A750 deletion): cells are grown in 12-well plates to 90% confluence and then incubated in low-serum (0.1% FBS) media for 16-18 h. Cells are then treated with varying concentration of test compounds (5, 1.25, 0.31, 0.078, 0.020 μM) or 0.5% DMSO in low-serum (0.1% FBS) media for 1 h. A431 cells are then stimulated with 50 ng/mL EGF for 15 mins. After treatment, cell monolayers are washed with cold PBS and immediately lysed by scraping into 50 μL cold Cell Extraction Buffer supplemented with Complete Protease inhibitors and phosphatase inhibitors. Lysate protein concentrations are determined by BCA assay and approximately 50 μg of each lysate were separated by 4-12% gradient SDS-PAGE transferred to nitrocellulose membrane and probed with specific antibodies. Phosphoprotein signals are visualized by western blot detection system or quantitated using Odyssey Infrared Imaging (Li-Cor Biosciences, Lincoln, Nebr.). To assess phospho-signaling, blots are immunoblotted with phospho and total antibodies for EGFR (Y1068), AKT, pS6RP and Erk1/2. Phospho-signals are normalized to total protein expression for each biomarker. Results are indicated as % DMSO control. Normalized data are fitted using a sigmoidal curve analysis program (Graph Pad Prism version 5) with variable Hill slope to determine the EC₅₀ values.

Antibodies: All primary antibodies are obtained from Cell Signaling (Danvers, Mass.) and used at 1:1000. Secondary antibodies are used at 1:20,000. Goat anti-mouse IgG IRDye 800CW antibody is obtained from LiCor Biosciences (Lincoln, Nebr.) and goat anti-rabbit IgG Alexa Fluor 680 is obtained from Invitrogen (Carlsbad, Calif.).

### Example C

### EGFR Cell Proliferation Assays

Cell Lines: A431 (WT), H1975 (L858R/T790M), PC9 (E746-A750 deletion): A431 cells were grown in DMEM (Invitrogen, Carlsbad, Calif.) supplemented with 10% FBS (HyClone, South Logan, Utah) and 1% Penicillin-Streptomycin (P/S, Lonza, Walkersville, Md.). H1975 cells were grown in RPMI 1640 (Invitrogen) supplemented with 10% FBS and 1% P/S. Culture Collection (Manassas, Va.), and PC-9 cells were obtained from Japan. All cells were maintained and propagated as monolayer cultures at 37° C. in a humidified 5% CO₂ incubator. All cells were cultured according to recommendations.

In order to profile the effect of EGFR inhibitors in various tumorigenic cell lines, the cell lines were tested in the cell proliferation assay that exhibit different EGFR mutation status. Cell proliferation was measured using the CellTiter-Glo® Luminescent Cell Viability Assay. The assay involved the addition of a single reagent (CellTiter-Glo® Reagent) directly to cells cultured in serum-supplemented medium. The assay used a one-step addition to induce cell lysis and generate a luminescent signal proportional to the amount of ATP present, which is directly proportional to the number of metabolically active cells present in culture.

Each compound evaluated was prepared as a DMSO stock solution (10 mM). Compounds were tested in duplicate on each plate, with an 11-point serial dilution curve (1:3 dilution). Compound treatment (50 μL) was added from the compound dilution plate to the cell plate. The highest compound concentration was 1 or 10 μM (final), with a 0.3% final DMSO (#D-5879, Sigma, St Louis, Mo.) concentration. Plates were then incubated at 37° C., 5% CO₂. After 3-5 days of compound treatment, CellTiter-Glo® Reagent (#G7573, Promega, Madison, Wis.) was prepared in one of two ways. If thawing a frozen aliquot of CellTiter-Glo® Reagent, the aliquot was thawed and equilibrated to RT prior to use while keeping it protected from light. Alternatively, new bottles of CellTiter-Glo® Buffer and CellTiter-Glo® Substrate were thawed and equilibrated to RT prior to use. CellTiter-Glo® Buffer (100 mL) was transferred into the amber bottle containing CellTiter-Glo® Substrate to reconstitute the lyophilized enzyme/substrate mixture, forming the CellTiter-Glo® Reagent. The reconstituted reagent was mixed by gently inverting the contents to obtain a homogeneous solution, and went into solution easily in less than 1 min. Any unused reconstituted CellTiter-Glo® Reagent was immediately aliquoted and frozen at −20° C., and protected from light. Cell plates were equilibrated at RT for approximately 30 mins. An equi-volume amount of CellTiter-Glo® Reagent (100 μL) was added to each well. Plates were mixed for 2 mins on an orbital shaker to induce cell lysis, and then were allowed to incubate at RT for 10 mins to stabilize the luminescent signal. Luminescence was recorded using the PerkinElmer EnVision Excite Multilabel Reader used for endpoint reading for luminescence detection (Waltham, Mass.). Data was analyzed using a four-parameter fit in Microsoft Excel.

Compounds of Formula (I) were active in this assay as provided in Table 2, where A=IC₅₀<50 nM; B=IC₅₀>50 nM and <300 nM; and C=IC₅₀≥300 nM.

TABLE 2

| | H1975 | PC9 | A431 |
|---|---|---|---|
| Example # | (nM) | (nM) | (nM) |
| 1 | A | A | C |
| 2 | A | A | B |
| 3 | A | A | B |
| 4 | A | A | B |
| 5 | A | A | B |
| 6 | B | A | C |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A compound of Formula (I):
wherein: 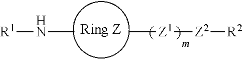
Ring Z is 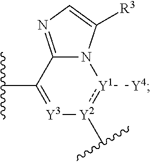
each [symbol] is independently a single or double bond;
Y¹ is C or N;
wherein Y² is N, the [symbol] between Y¹ and Y² is a single bond, Y¹ is C, the -----bond to Y⁴ is a double bond and Y⁴ is O; or
wherein Y² is C, the [symbol] between Y¹ and Y² is a double bond, Y¹ is N, the -----bond is absent and Y⁴ is absent; or
wherein Y² is C, the [symbol] between Y¹ and Y² is a double bond, Y¹ is C, the -----bond to Y⁴ is a single bond and Y⁴ is selected from the group consisting of hydrogen, halogen, an optionally substituted C₁₋₄ alkyl, an optionally substituted cycloalkyl, an optionally substituted alkoxy, an optionally substituted mono-substituted amine and an optionally substituted disubstituted amine;
Y³ is CR^{1A} or N;
R¹ is an optionally substituted aryl or an optionally substituted heteroaryl;
R² is selected from the group consisting of a substituted C₄-C₁₀ cycloalkyl, a substituted aryl, a substituted heteroaryl and a substituted heterocyclyl, and wherein R² is substituted with an activated alkenyl;
R³ is selected from the group consisting of hydrogen, halogen, an optionally substituted C₁₋₄ alkyl, an optionally substituted C₃-C₁₀ cycloalkyl, an optionally substituted alkoxy, an optionally substituted mono-substituted sulfenyl, an optionally substituted mono-substituted amine and an optionally substituted disubstituted amine;
R^{1A} is selected from the group consisting of hydrogen, halogen, an optionally substituted C₁₋₄ alkyl, an optionally substituted cycloalkyl, an optionally substituted alkoxy, an optionally substituted mono-substituted amine and an optionally substituted disubstituted amine;
Z¹ is O, S or NH;
Z² is (CR^{2A}R^{2B})n;
R^{2A} and R^{2B} are independently selected from the group consisting of hydrogen, halogen, an optionally substituted C₁₋₄ alkyl, an optionally substituted C₁₋₄ alkoxy and an optionally substituted C₁₋₄ haloalkyl;
m is 0 or 1; and
n is 0, 1, 2 or 3.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A compound of Formula (I): 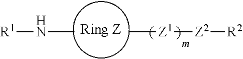
wherein:
Ring Z is 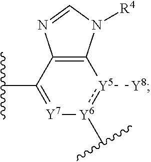
each [symbol] is independently a single or double bond;
Y⁵ is C or N;
wherein Y⁶ is N, the [symbol] between Y⁵ and Y⁶ is a single bond, Y⁵ is C, the -----bond to Y⁸ is a double bond and Y⁸ is O; or
wherein Y⁶ is C, the [symbol] between Y⁵ and Y⁶ is a double bond, Y⁵ is C, the -----bond to Y⁸ is a single bond and Y⁸ is selected from the group consisting of hydrogen, halogen, an optionally substituted C₁₋₄ alkyl, an optionally substituted cycloalkyl, an optionally substituted alkoxy, an optionally substituted mono-substituted amine and an optionally substituted disubstituted amine;
Y⁷ is CR^{1B} or N;
R¹ is an optionally substituted aryl or an optionally substituted heteroaryl;
R² is selected from the group consisting of a substituted C₄-C₁₀ cycloalkyl, a substituted aryl, a substituted heteroaryl and a substituted heterocyclyl, and wherein R² is substituted with an activated alkenyl;
R⁴ is selected from the group consisting of hydrogen, halogen, an optionally substituted C₁₋₄ alkyl, an optionally substituted C₃-C₁₀ cycloalkyl, an optionally substituted alkoxy, an optionally substituted mono-substituted sulfenyl, an optionally substituted mono-substituted amine and an optionally substituted disubstituted amine;
R^{1B} is selected from the group consisting of hydrogen, halogen, an optionally substituted C₁₋₄ alkyl, an optionally substituted cycloalkyl, an optionally substituted alkoxy, an optionally substituted mono-substituted amine and an optionally substituted disubstituted amine;
Z¹ is O, S or NH;
Z² is (CR^{2A}R^{2B})n;
R^{2A} and R^{2B} are independently selected from the group consisting of hydrogen, halogen, an optionally substituted C₁₋₄ alkyl, an optionally substituted C₁₋₄ alkoxy and an optionally substituted C₁₋₄ haloalkyl;
m is 0 or 1; and
n is 0, 1, 2 or 3.

19. (canceled)

20. (canceled)

21. (canceled)

22. (canceled)

23. A compound of Formula (I): 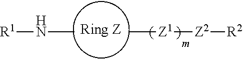
wherein:
Ring Z is 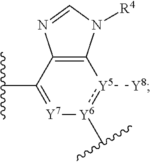
each [symbol] is independently a single or double bond;
Y⁵ is C or N;
wherein Y⁶ is C, the [symbol] between Y⁵ and Y⁶ is a double bond, Y⁵ is N, the -----bond is absent and Y⁸ is absent; [symbol]
Y⁷ is CR^{1B} or N;
R¹ is an optionally substituted aryl or an optionally substituted heteroaryl;
R² is selected from the group consisting of a substituted C₄-C₁₀ cycloalkyl and a substituted heterocyclyl, and wherein R² is substituted with an activated alkenyl;
R⁴ is selected from the group consisting of hydrogen, halogen, an optionally substituted C₁₋₄ alkyl, an optionally substituted C₃-C₁₀ cycloalkyl, an optionally substituted alkoxy, an optionally substituted mono-substituted sulfenyl, an optionally substituted mono-substituted amine and an optionally substituted disubstituted amine;
R^{1B} is selected from the group consisting of hydrogen, halogen, an optionally substituted C₁₋₄ alkyl, an optionally substituted cycloalkyl, an optionally substituted alkoxy, an optionally substituted mono-substituted amine and an optionally substituted disubstituted amine;
Z¹ is O, S or NH;
Z² is (CR^{2A}R^{2B})n;
R^{2A} and R^{2B} are independently selected from the group consisting of hydrogen, halogen, an optionally substituted C₁₋₄ alkyl, an optionally substituted C₁₋₄ alkoxy and an optionally substituted C₁₋₄ haloalkyl;
m is 0 or 1; and
n is 0, 1, 2 or 3; and
provided that when Ring Z is 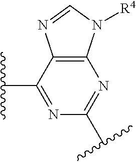
then R⁴ cannot be an unsubstituted C₁₋₄ alkyl, a C₁₋₄ alkyl substituted with hydroxy, an unsubstituted C₃-C₄ cycloalkyl or a C₃-C₄ cycloalkyl substituted with an unsubstituted C₁₋₄ alkyl; and
provided that a compound of Formula (I), or a pharmaceutically acceptable salt, cannot be selected from the group consisting of:

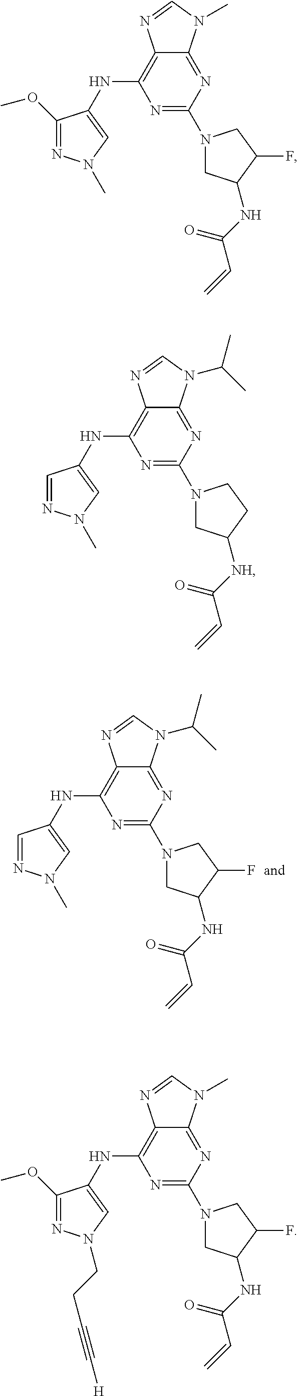

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The compound of claim 1, wherein R¹ is an optionally substituted heteroaryl.

37. (canceled)

38. (canceled)

39. (canceled)

40. The compound of claim 1, wherein R² is a substituted C₄-C₁₀ cycloalkyl, a substituted aryl or a substituted heterocyclyl.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The compound of claim 1, wherein m is 0; and n is 0; or wherein m is 0; Z² is (CH₂)n; and n is 1.

58. (canceled)

59. (canceled)

60. (canceled)

61. (canceled)

62. (canceled)

63. (canceled)

64. (canceled)

65. (canceled)

66. (canceled)

67. The compound of claim 1 selected from the group consisting of: 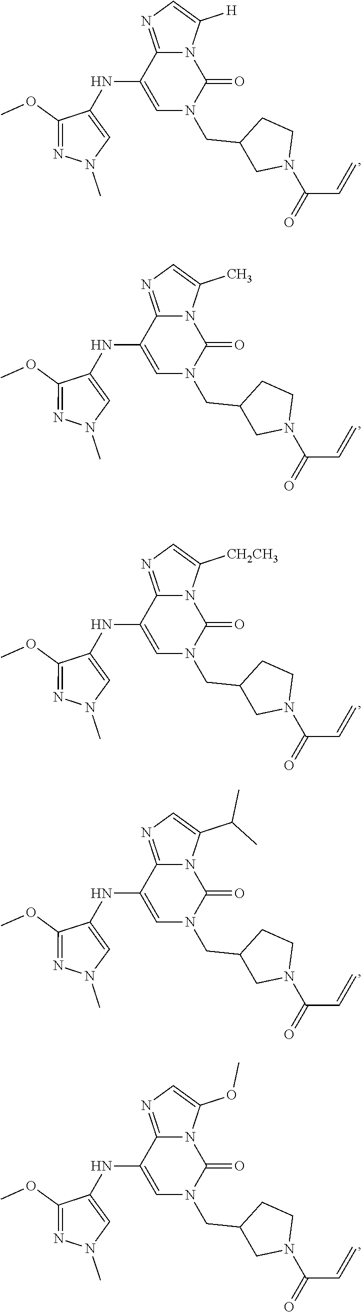 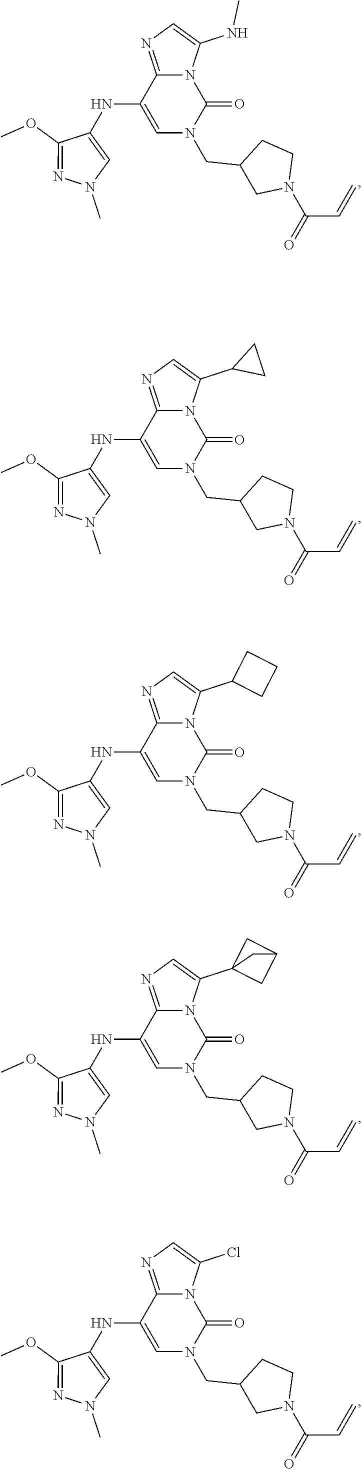 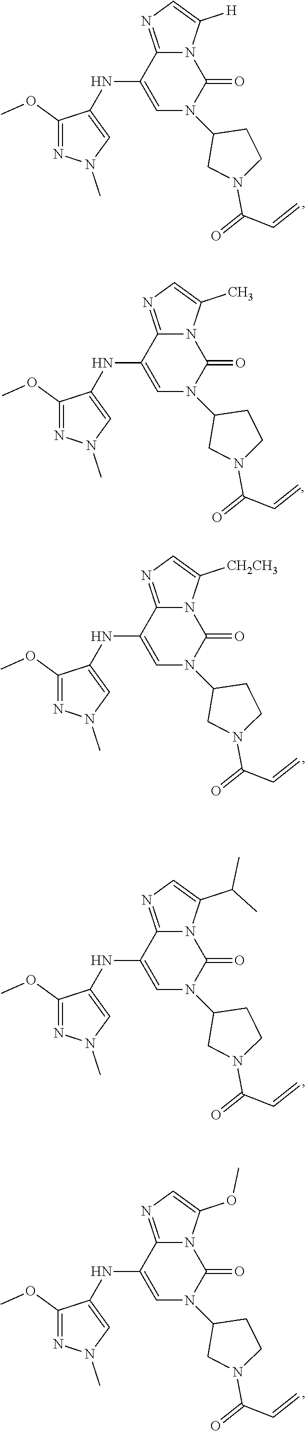 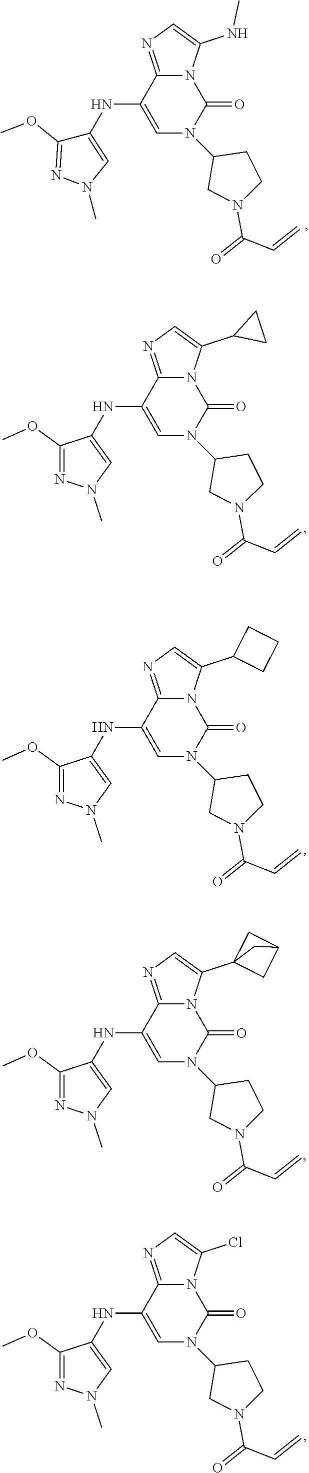 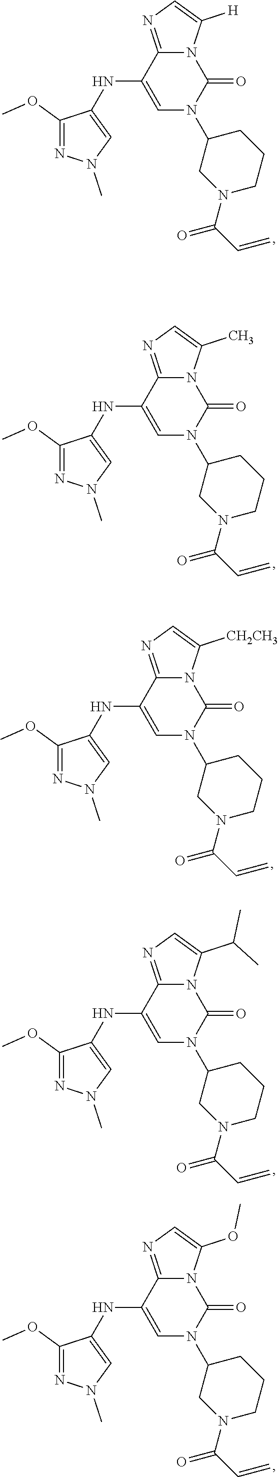 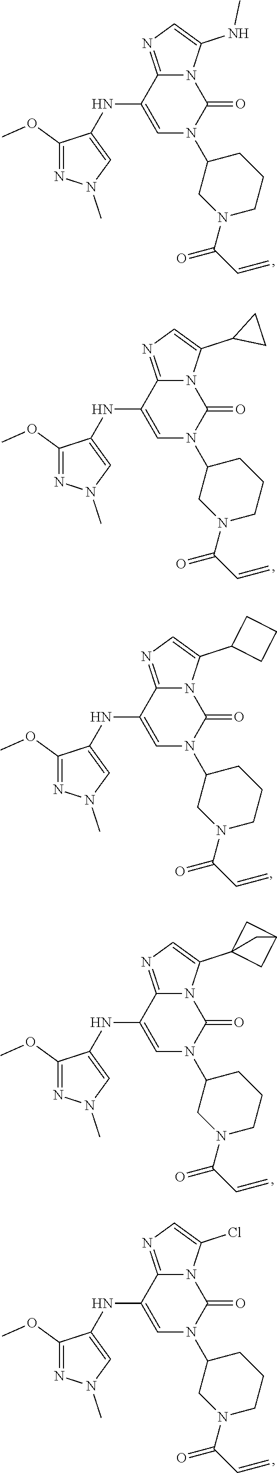 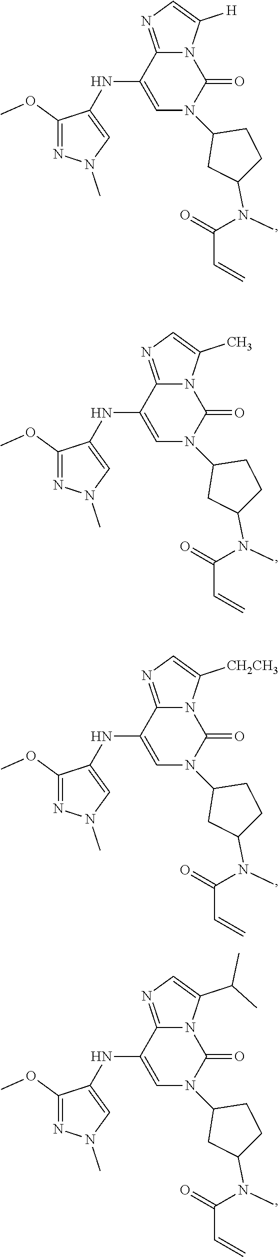 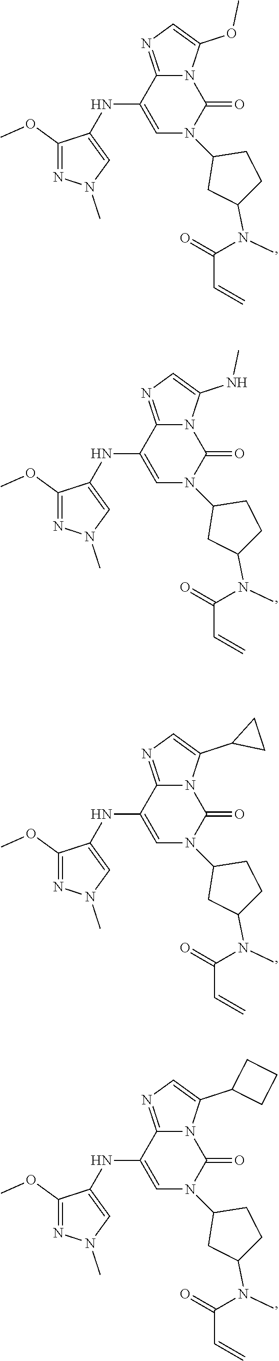 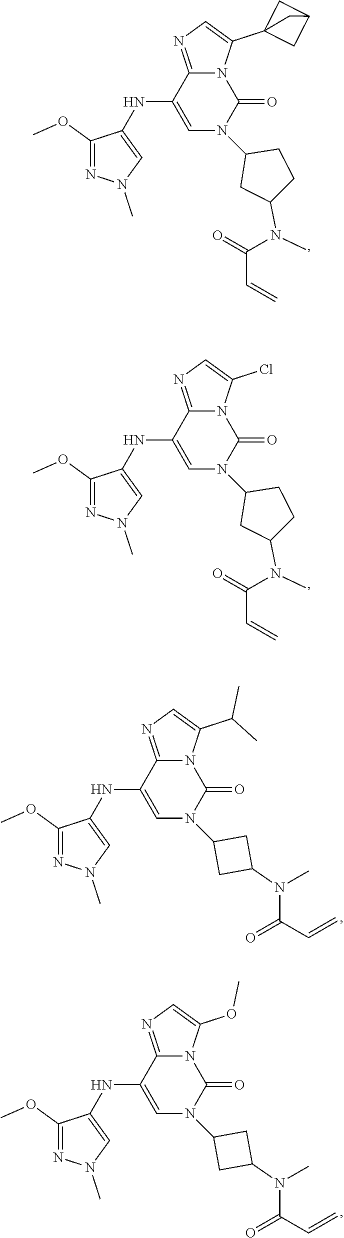 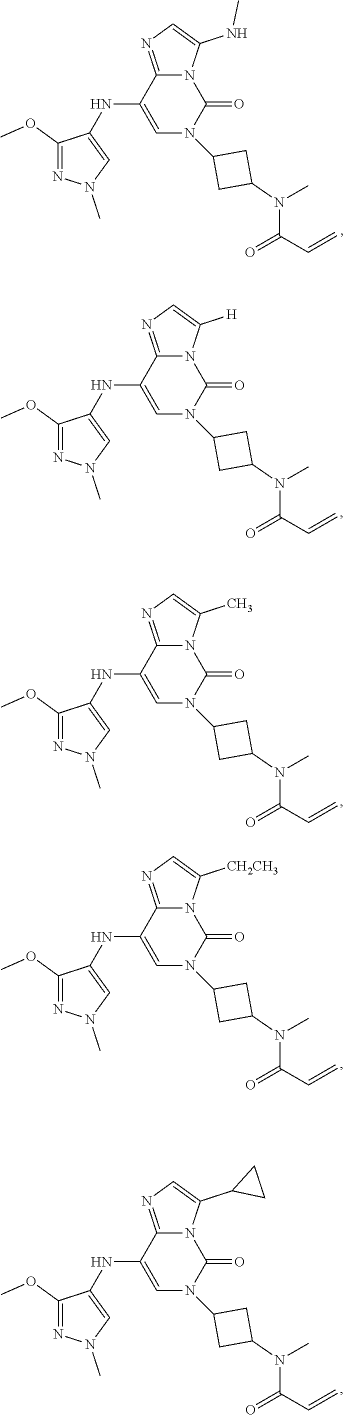 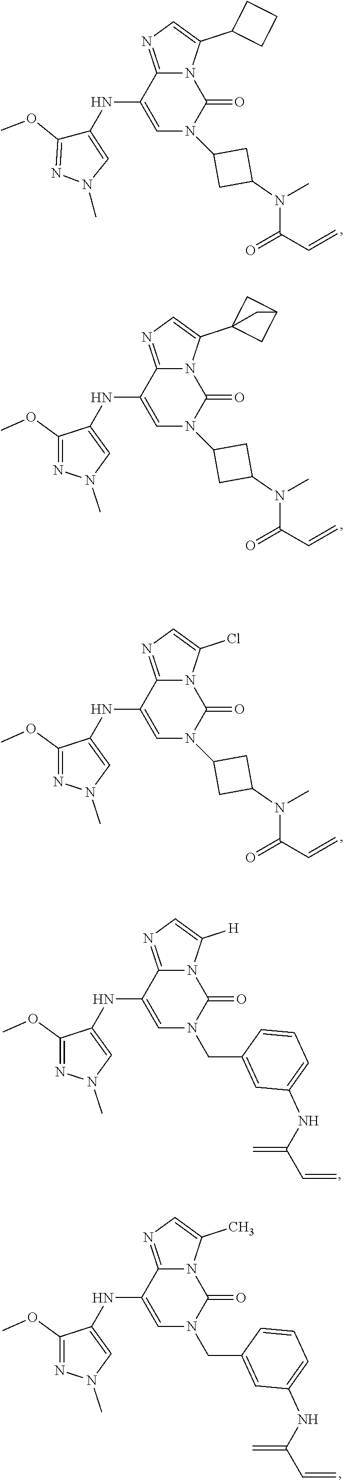 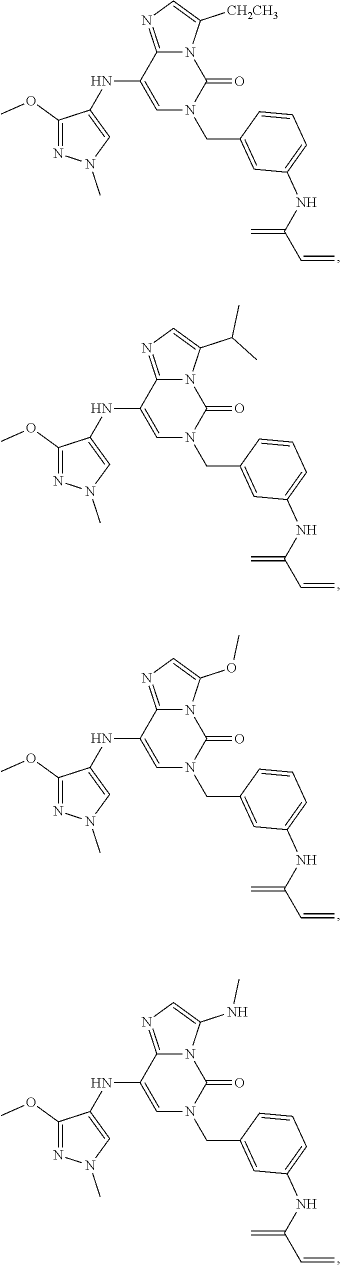 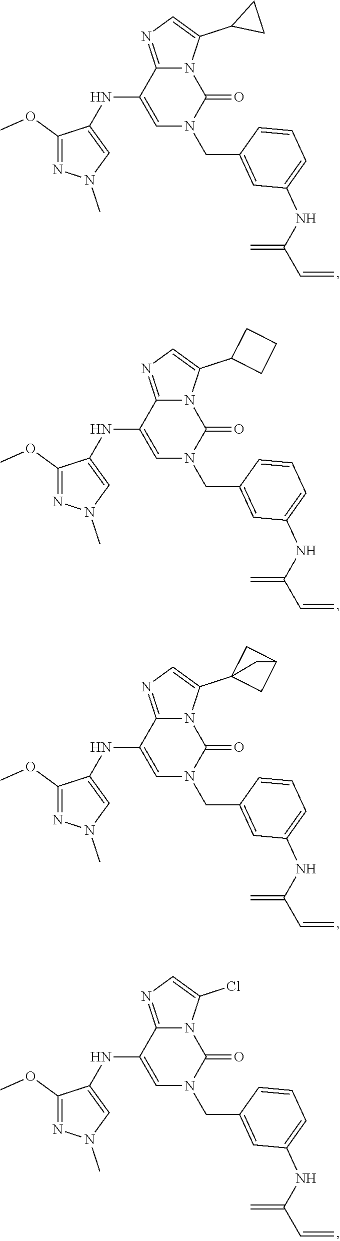 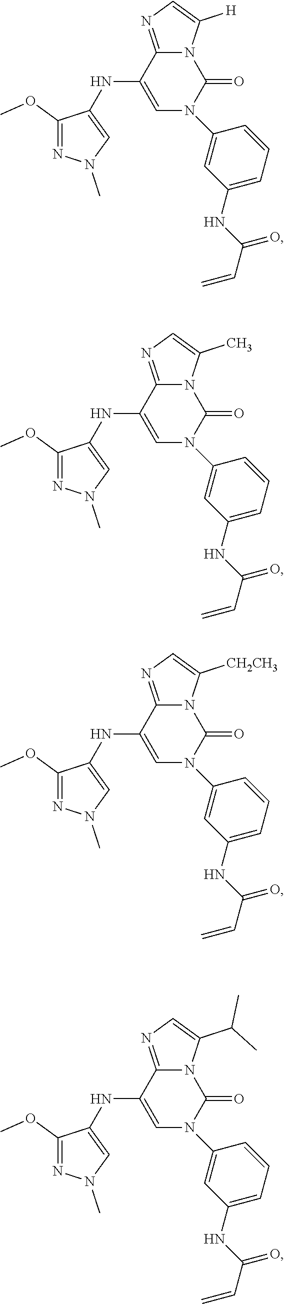 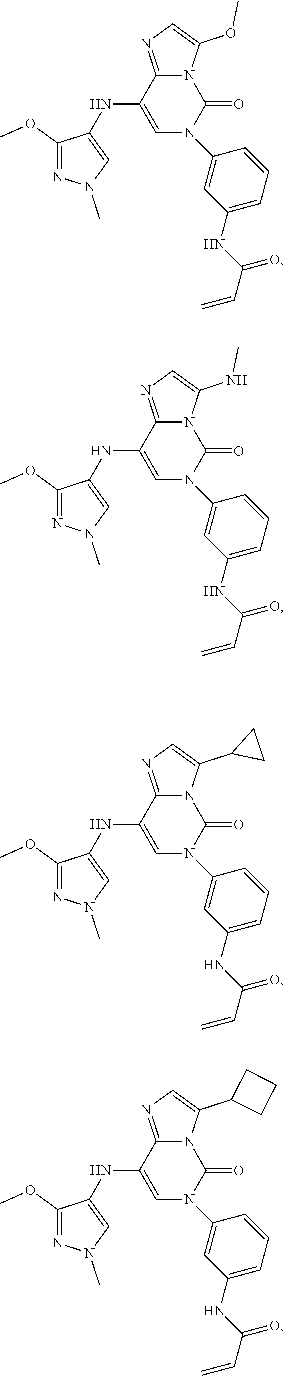 [SMILES: C=CC(=O)Nc1cccc(-n2cc(Nc3cn(C)nc3OC)c3ncc(OC)n3c2=O)c1 ; C=CC(=O)Nc1cccc(-n2cc(Nc3cn(C)nc3OC)c3ncc(NC)n3c2=O)c1 ; C=CC(=O)Nc1cccc(-n2cc(Nc3cn(C)nc3OC)c3ncc(C4CC4)n3c2=O)c1 ; C=CC(=O)Nc1cccc(-n2cc(Nc3cn(C)nc3OC)c3ncc(C4CCC4)n3c2=O)c1] 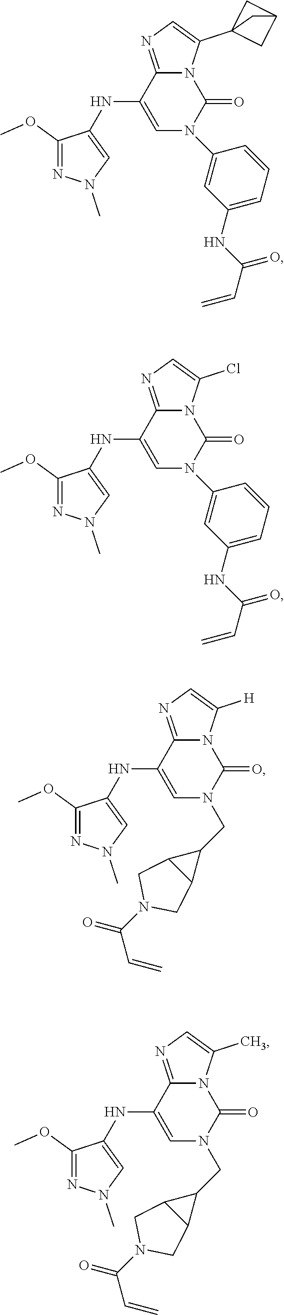 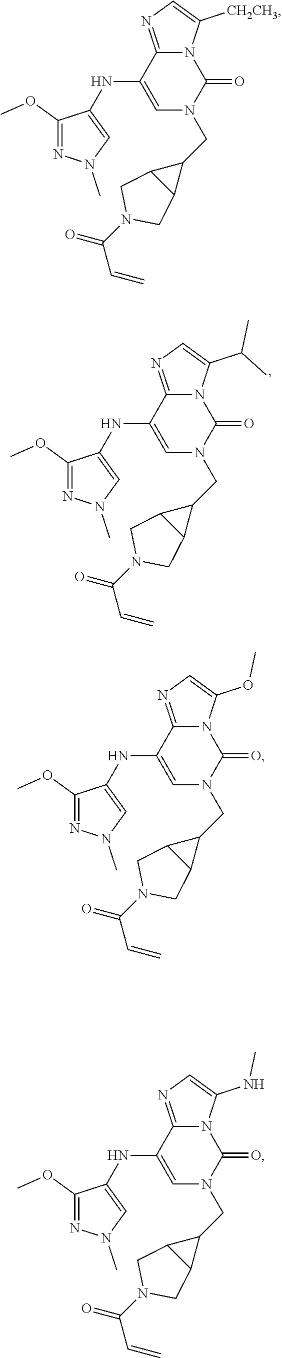 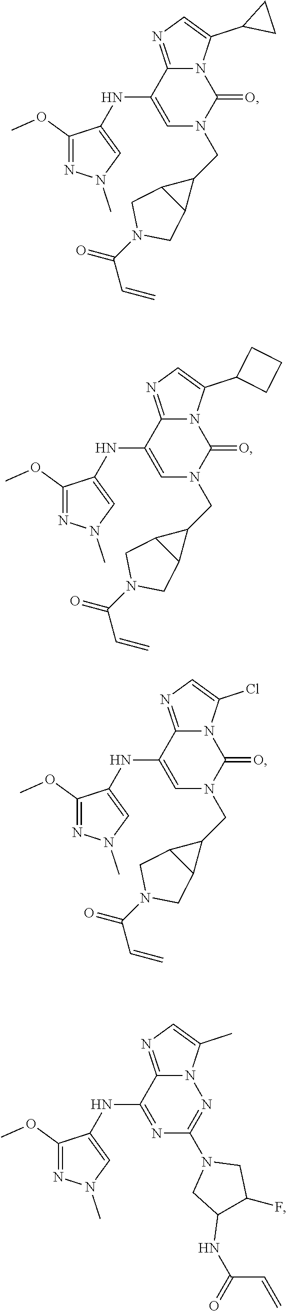 [SMILES: C=CC(=O)N1CC2C(C1)C2Cn1cc(Nc2cn(C)nc2OC)c2ncc(C3CC3)n2c1=O ; C=CC(=O)N1CC2C(C1)C2Cn1cc(Nc2cn(C)nc2OC)c2ncc(C3CCC3)n2c1=O ; C=CC(=O)N1CC2C(C1)C2Cn1cc(Nc2cn(C)nc2OC)c2ncc(Cl)n2c1=O ; C=CC(=O)NC1CN(c2nc(Nc3cn(C)nc3OC)c3ncc(C)n3n2)CC1F] 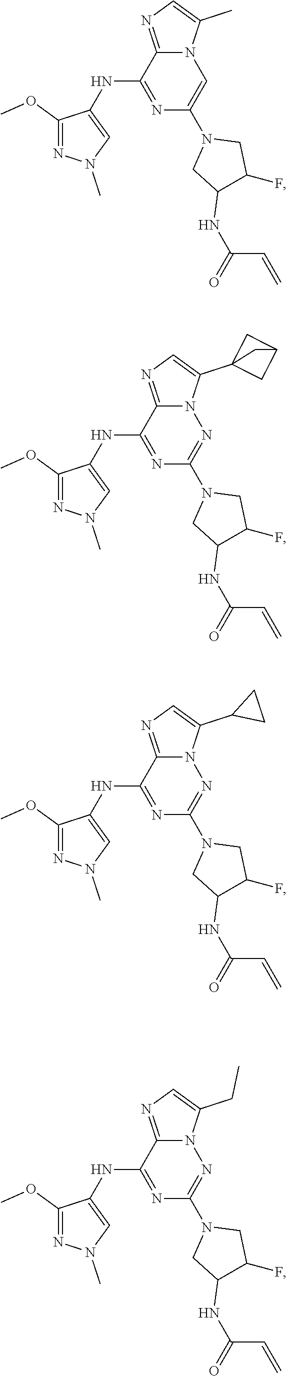 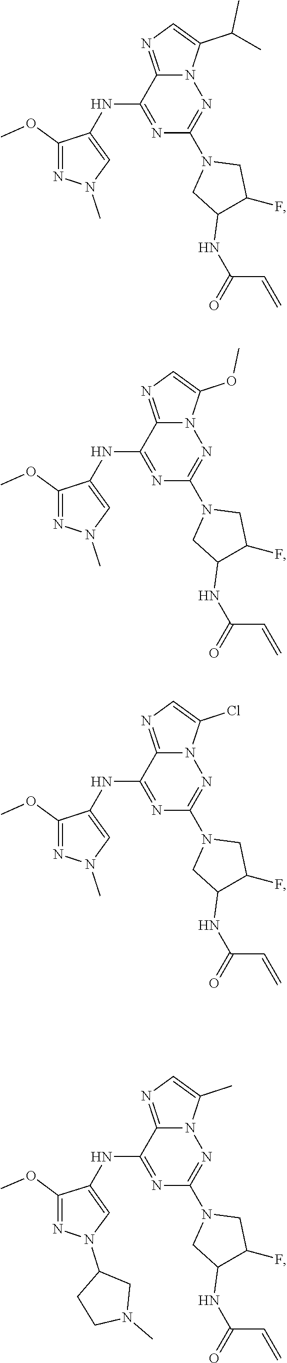 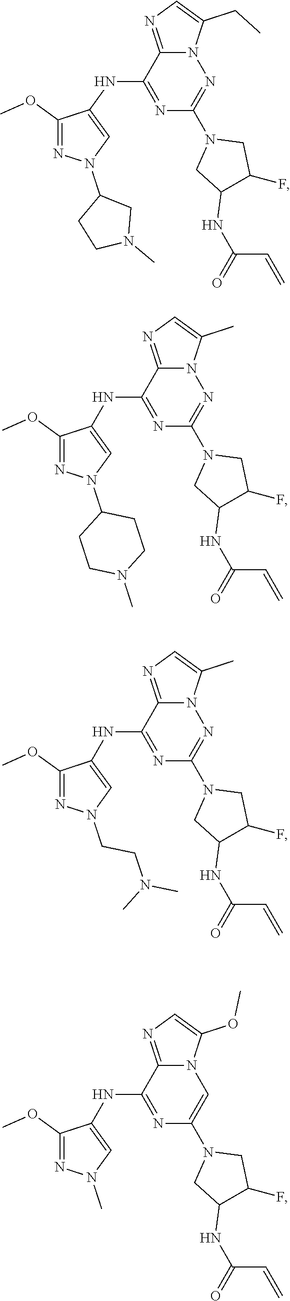 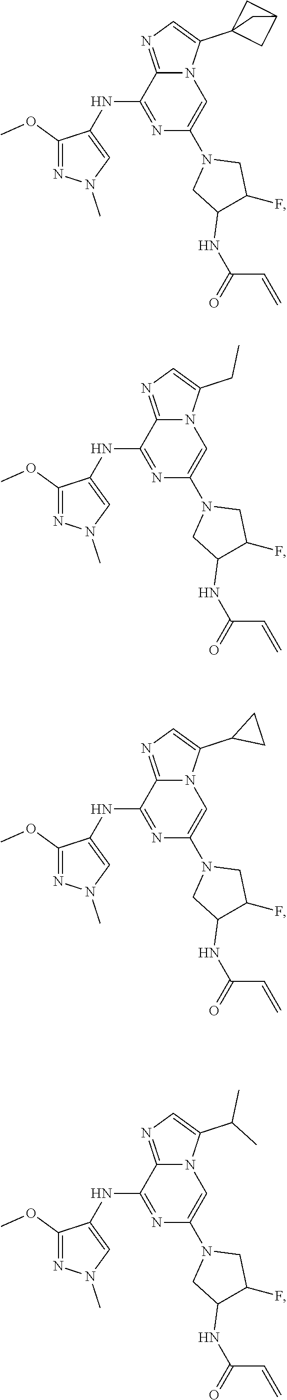 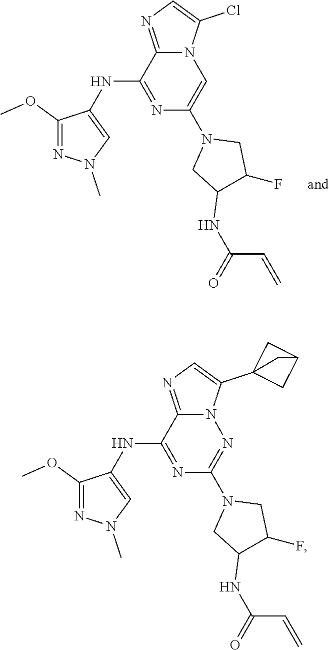
or a pharmaceutically acceptable salt of any of the foregoing.

68. The compound of claim 18 selected from the group consisting of: 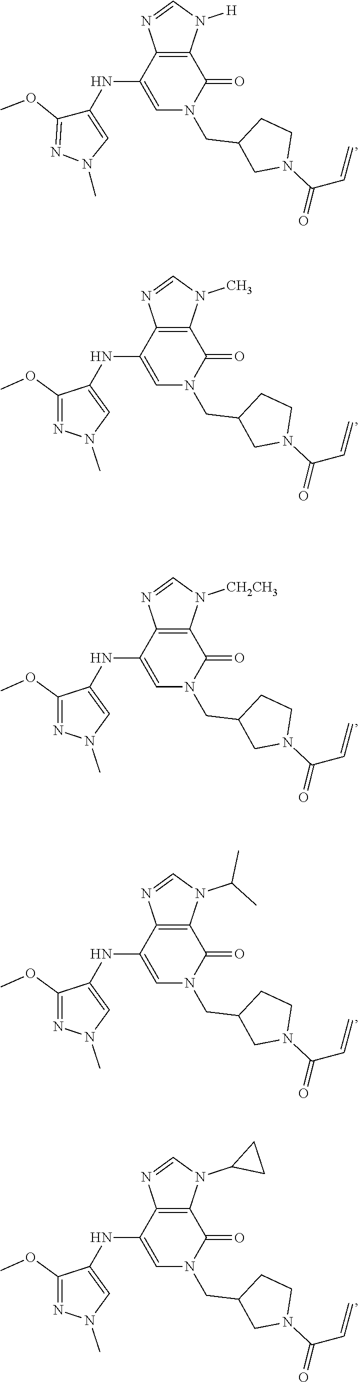 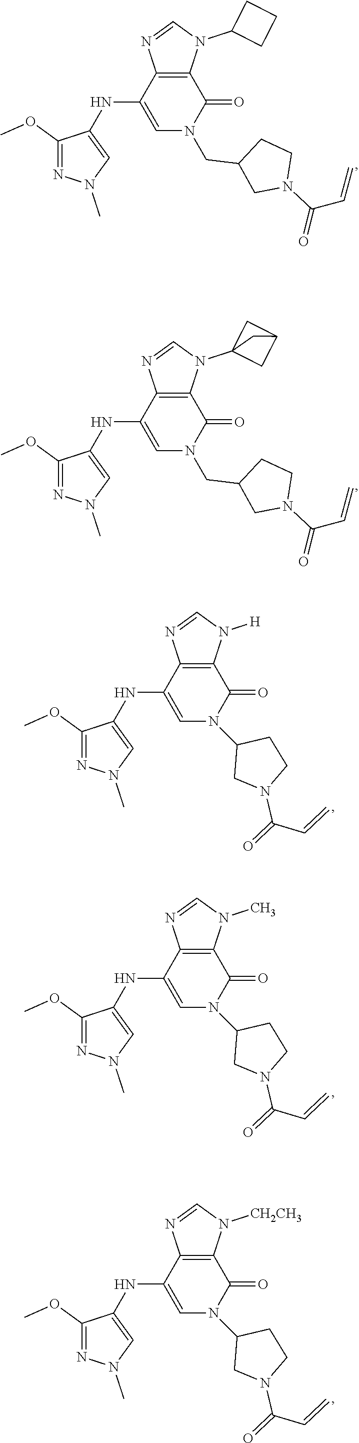 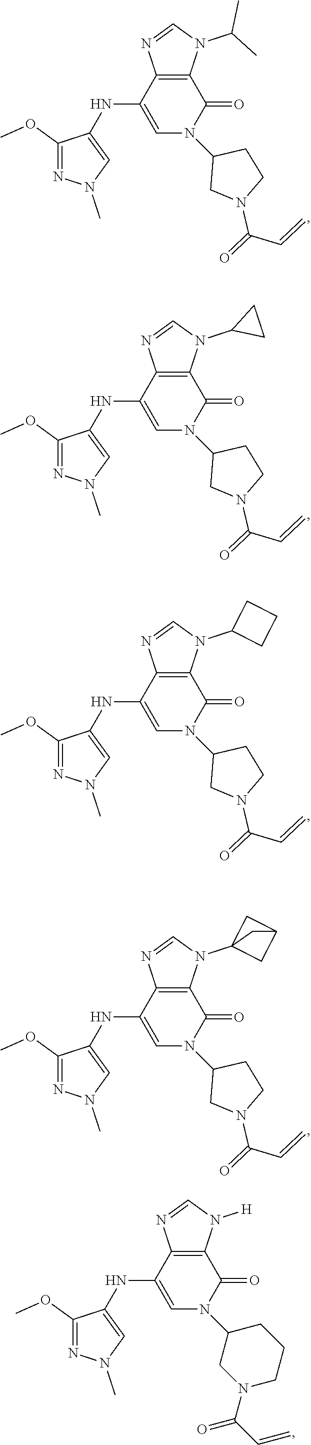 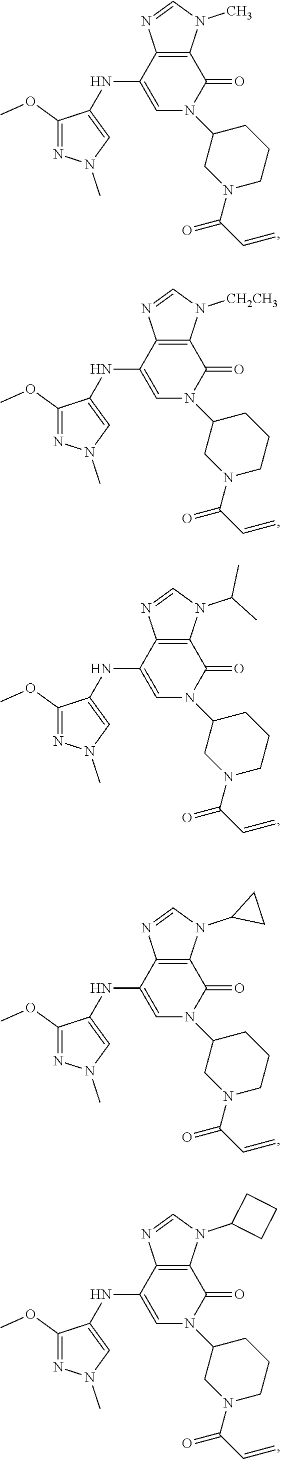 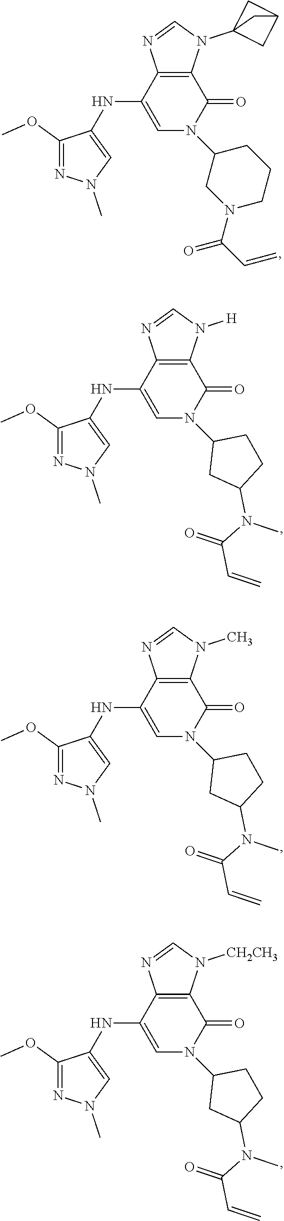 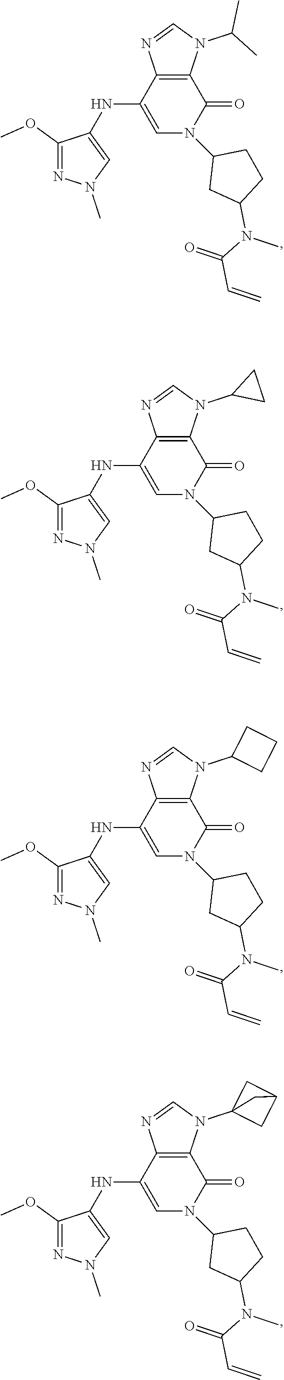 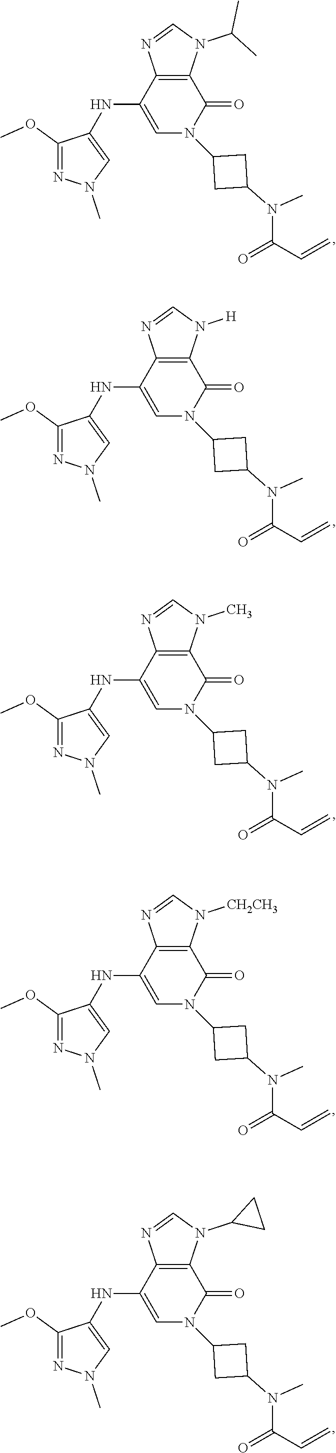 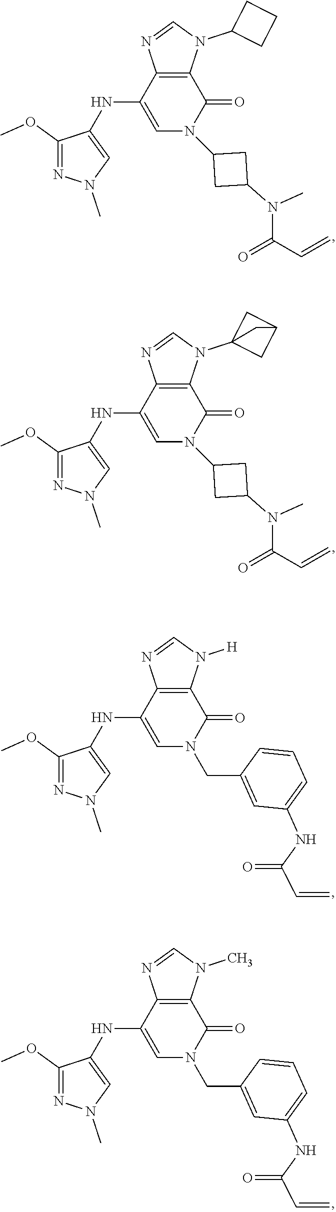 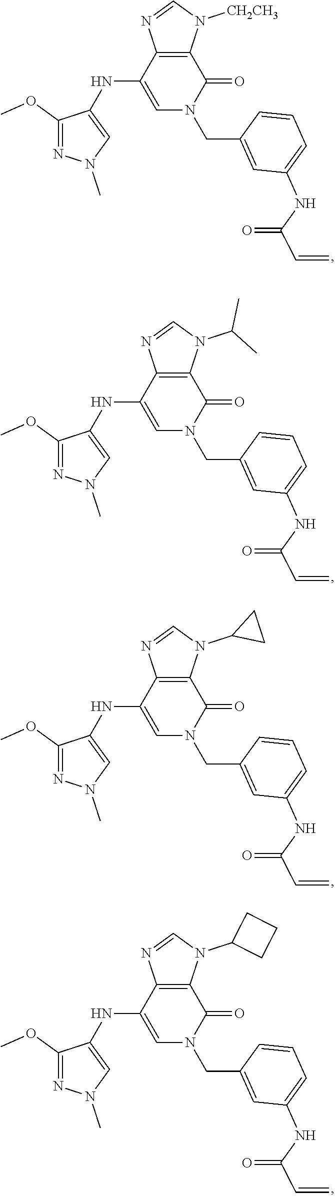 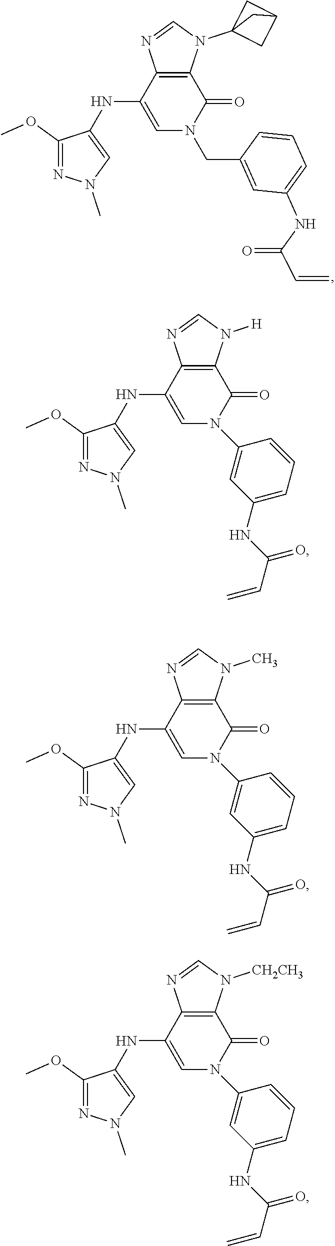 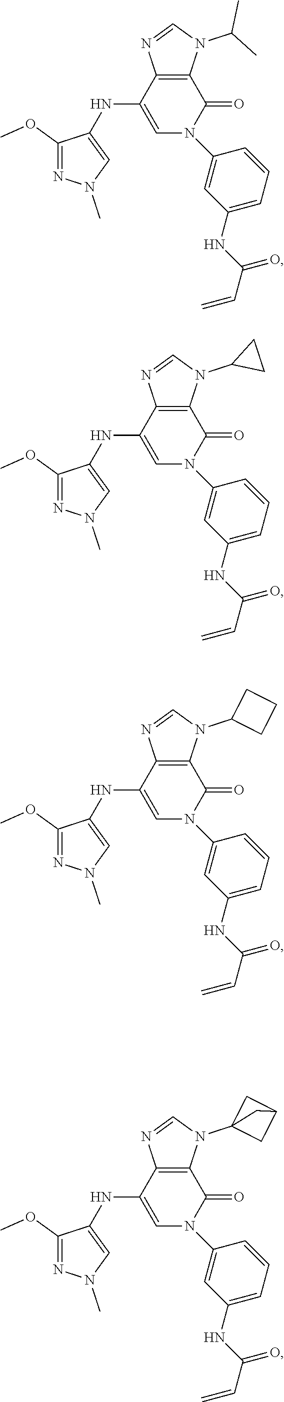 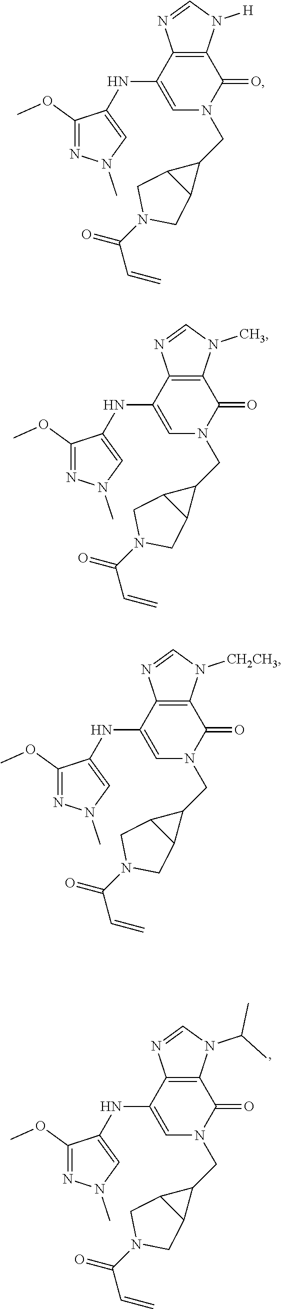 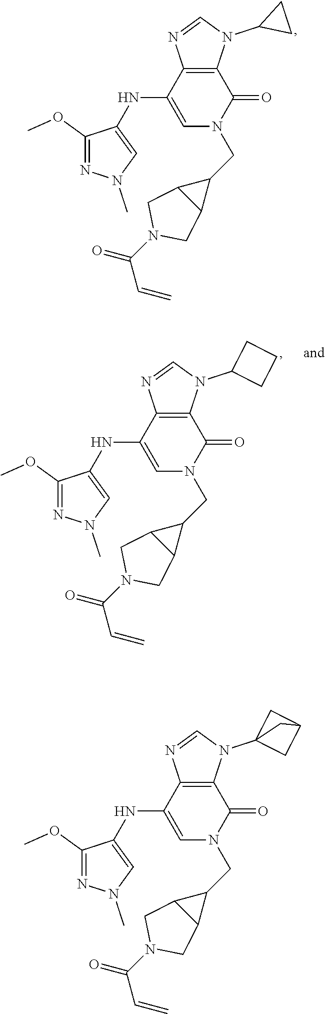 [SMILES: C=CC(=O)N1CC2C(C1)C2Cn1cc(Nc2cn(C)nc2OC)c2ncn(C3CC3)c2c1=O ; C=CC(=O)N1CC2C(C1)C2Cn1cc(Nc2cn(C)nc2OC)c2ncn(C3CCC3)c2c1=O ; C=CC(=O)N1CC2C(C1)C2Cn1cc(Nc2cn(C)nc2OC)c2ncn(C34CC(C3)C4)c2c1=O]
or a pharmaceutically acceptable salt of any of the foregoing.

69. A pharmaceutical composition comprising an effective amount of a compound of claim 1, or a pharmaceutically acceptable salt thereof, and a pharmaceutically acceptable carrier, diluent, excipient, or combination thereof.

70. A method for ameliorating or treating a cancer comprising administering an effective amount of a compound of claim 1, or a pharmaceutically acceptable salt thereof, wherein the cancer is selected from the group consisting of a lung cancer, a pancreatic cancer, a colon cancer, a breast cancer, a prostate cancer, a head and neck cancer, an ovarian cancer, a brain cancer and a kidney carcinoma.

71. A method for inhibiting replication of a malignant growth or a tumor comprising contacting the growth or the tumor with an effective amount of a compound of claim 1, or a pharmaceutically acceptable salt thereof, wherein the malignant growth or tumor is due to a cancer is selected from the group consisting of a lung cancer, a pancreatic cancer, a colon cancer, a breast cancer, a prostate cancer, a head and neck cancer, an ovarian cancer, a brain cancer and a kidney carcinoma.

72. A method for ameliorating or treating a cancer comprising contacting a malignant growth or a tumor with an effective amount of a compound of claim 1, or a pharmaceutically acceptable salt thereof, wherein the malignant growth or tumor is due to a cancer is selected from the group consisting of a lung cancer, a pancreatic cancer, a colon cancer, a prostate cancer, a head and neck cancer, an ovarian cancer, a brain cancer and a kidney carcinoma.

73. A method for inhibiting the activity of EGFR comprising providing an effective amount of a compound of claim 1, or a pharmaceutically acceptable salt thereof, to a sample comprising a cancer cell, wherein the cancer cell is selected from the group consisting of a lung cancer cell, a pancreatic cancer cell, a colon cancer cell, a breast cancer cell, a prostate cancer cell, a head and neck cancer cell, an ovarian cancer cell, a brain cancer cell and a kidney carcinoma cell.

74. A method for inhibiting the activity of EGFR comprising providing an effective amount of a compound of claim 1, or a pharmaceutically acceptable salt thereof, to a subject in need thereof, wherein the EGFR has an acquired EGFR T790M mutation.

75. (canceled)

76. (canceled)

77. (canceled)

78. (canceled)

79. The compound of claim 18, wherein R¹ is an optionally substituted heteroaryl.

80. The compound of claim 18, wherein R² is a substituted C₄-C₁₀ cycloalkyl, a substituted aryl or a substituted heterocyclyl.

81. The compound of claim 18, wherein m is 0; and n is 0; or wherein m is 0; Z² is (CH₂)n; and n is 1.

82. The compound of claim 23, wherein R¹ is an optionally substituted heteroaryl.

83. The compound of claim 23, wherein R² is a substituted C₄-C₁₀ cycloalkyl, a substituted aryl or a substituted heterocyclyl.

84. The compound of claim 23, wherein m is 0; and n is 0; or wherein m is 0; Z² is (CH₂)n; and n is 1.

85. The compound of claim 23, having the structure of 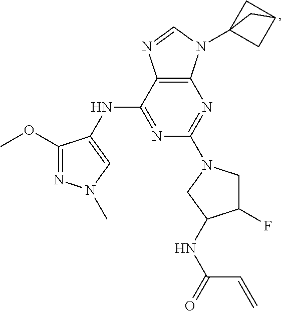 [SMILES: C=CC(=O)NC1CN(c2nc(Nc3cn(C)nc3OC)c3ncn(C45CC(C4)C5)c3n2)CC1F]
or a pharmaceutically acceptable salt thereof.
